SEQUENCE LISTING (35 sequences)

SEQ ID NO: 1 - protein, 809 aa, Homo sapiens
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 2 - protein, 776 aa, Homo sapiens
AGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 3 - protein, 288 aa, Homo sapiens
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLS

SEQ ID NO: 4 - protein, 255 aa, Homo sapiens
AGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLS

SEQ ID NO: 5 - protein, 104 aa, Homo sapiens
GKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLS

SEQ ID NO: 6-16 - skipped (11 entries)

SEQ ID NO: 17 - protein, 23 aa, Artificial Sequence
  source: /note=“Description of Artificial Sequence: Synthetic peptide”
MAPFEPLASGILLLLWLIAPSRA

SEQ ID NO: 18 - skipped

SEQ ID NO: 19 - DNA, 32 bp, Artificial Sequence
  source: /note=“Description of Artificial Sequence: Synthetic primer”
ggaggaacatatggcgggcggtcatggtgatg

SEQ ID NO: 20 - DNA, 34 bp, Artificial Sequence
  source: /note=“Description of Artificial Sequence: Synthetic primer”
ctaggatccttaccgttgatctttaagttcttcc

SEQ ID NO: 21 - DNA, 34 bp, Artificial Sequence
  source: /note=“Description of Artificial Sequence: Synthetic primer”
ctagaagtccatggcgtcccaaggcaccaaacgg

SEQ ID NO: 22 - DNA, 33 bp, Artificial Sequence
  source: /note=“Description of Artificial Sequence: Synthetic primer”
ctagagctcattgtcgtactcttctgcattgtc

SEQ ID NO: 23 - protein, 14 aa, Artificial Sequence
  source: /note=“Description of Artificial Sequence: Synthetic peptide”
GSPGISGGGGGILE

SEQ ID NO: 24-32 - skipped (9 entries)

SEQ ID NO: 33 - DNA, 4 bp, Artificial Sequence
  source: /note=“Description of Artificial Sequence: Synthetic oligonucleotide”
tata

SEQ ID NO: 34 - RNA, 6 nt, Artificial Sequence
  source: /note=“Description of Artificial Sequence: Synthetic oligonucleotide”
aauaaa

SEQ ID NO: 35 - protein, 6 aa, Artificial Sequence
  source: /note=“Description of Artificial Sequence: Synthetic 6xHis tag”
HHHHHH

(19) United States
(12) Patent Application Publication
(10) Pub. No.: US 2013/0115237 A1
(43) Pub. Date: May 9, 2013

(54) THERAPEUTIC IMMUNIZATION IN HIV INFECTED SUBJECTS TO AUGMENT ANTIRETROVIRAL TREATMENT

(71) Applicants: Yichen Lu, Wellesley, MA (US); Huyen Cao, San Francisco, CA (US)

(72) Inventors: Yichen Lu, Wellesley, MA (US); Huyen Cao, San Francisco, CA (US)

(73) Assignee: VACCINE TECHNOLOGIES, INCORPORATED, Wellesley, MA (US)

(21) Appl. No.: 13/703,174

(22) PCT Filed: Jun. 9, 2011
(86) PCT No.: PCT/US11/39797

§ 371 Date: Dec. 10, 2012

Related U.S. Application Data
(60) Provisional application No. 61/353,176, filed on Jun. 9, 2010.

(51) Int. Cl. A61K 39/12; A61K 39/08

(52) U.S. Cl. 424/190.1

(57) ABSTRACT

The present invention generally relates to HIV compositions and methods of use. One aspect of the present invention relates to a composition comprising a pharmaceutically acceptable carrier and an antigen preparation, the antigen preparation comprising an HIV polypeptide or fragment thereof and a Bacillus anthracis lethal factor (LF) polypeptide, such as a LFn polypeptide. In some embodiments, the LF polypeptide can be fused or otherwise associated with the HIV polypeptide. Other aspect of the present invention relate to use of vaccine comprising a HIV polypeptide and a Bacillus anthracis lethal factor (LF) polypeptide in methods to enhance efficacy traditional antiretroviral therapy.

### CROSS REFERENCE TO RELATED APPLICATIONS

This application claims benefit under 35 U.S.C. 119(e) of U.S. Provisional Patent Application Ser. No. 61/353,176 filed on Jun. 9, 2010, the contents of which is incorporated herein in its entity by reference.

### FIELD OF THE INVENTION

The present application is generally directed to compositions and methods for vaccinations against HIV virus, and in particular delivering an exogenous HIV virus protein to the cytosol of a cell, and methods for use in conjunction with conventional retroviral therapy to improve retroviral therapy.

### BACKGROUND OF INVENTION

An estimated 25 million people with HIV infection reside in sub-Saharan Africa. In resource poor settings, restricted treatment options and the cost of alternative treatment regimens are likely to magnify the limitations of antiretroviral treatment (ART) programs¹. Uganda's current HIV infection's rate of 6-10% remains unacceptably high. The majority of ART regimens available in Uganda are restricted to low-cost, fixed-dose combination drugs² and options for second line treatment remain limited. Antiretroviral treatment can successfully reduce plasma HIV-1 RNA levels to <50 copies/mL³⁻⁵ although the proportion of people who virologically respond is lower for those who start therapy with more advanced immunosuppression and higher viral loads⁶. ART has been linked to abnormalities of body fat metabolism and distribution, hyperlipidemia, insulin resistance, hyperglycemia, and lactic acidosis⁷⁻¹¹ requiring regimen change in 40% of individuals within 1 year of starting treatment^{12,13}.

Therapeutic interventions that enhance immunologic function with concurrent ART may improve the long-term outcome of HIV infection¹⁴. Immune recovery following appropriate ART is often incomplete and fail to elicit responses associated with protection from disease progression¹⁵⁻¹⁸. This failure has been associated with dysfunctional T cell responses¹⁹⁻²¹. Therefore, boosting the immune responses via therapeutic intervention may significantly delay or inhibit progression to AIDS. Therapeutic vaccinations in rhesus macaques have demonstrated that immunity can be elicited, leading to lower viral load²². Prior studies have shown a reduction in plasma viremia in HIV infected individuals²³⁻²⁵ and evidence of enhanced HIV specific T cell responses after immunizations²³⁻²⁶. However, no HIV vaccine has received clinical approval.

LFn-p24C consists of a detoxified anthrax-derived polypeptide, called lethal factor n-terminus (LFn), fused to the HIV-gag protein p24. In vivo tests of this recombinant protein delivery approach have demonstrated cellular delivery of peptides that stimulate potent immune responses²³⁻²⁵.

### SUMMARY OF THE INVENTION

The present invention relates generally to therapeutic compositions comprising a HIV polypeptide or peptide and a LFn protein to effectively deliver HIV to the cytosol of the cell to elicit a cytotoxic lymphocyte response (CTL) to the HIV immunogen to increase immunity against HIV of HIV infected individuals during antiretroviral treatment.

As disclosed herein, the inventors demonstrate use of a LFn-p24C vaccine as a therapeutic immunogen in a two-phase, open label trial. Phase 1A evaluated the safety of the vaccine candidate followed, and a Phase 1B study was used to demonstrate that the LFn-p24C vaccine composition can be used for a short interruption of the duration of conventional anti-retroviral treatment.

Accordingly, the inventors demonstrate clinical efficacy of a vaccine HIV LFn-p24 vaccine to increase and improve the efficacy traditional antiviral therapy in a therapeutic vaccine trial in Uganda, Africa. This open label Phase I trial was designed to assess the safety, tolerability and immunogenicity of LFn-p24C as a therapeutic HIV-1 vaccine candidate. Thirty healthy, HIV positive volunteers receiving a stable regimen of antiretroviral therapy (ART) with CD4+ T cell counts >400 were recruited for the safety evaluation of LFn-p24C. This vaccine comprises an anthrax-derived polypeptide, called lethal factor N-terminus (LFn), fused to the subtype C HIV gag protein p24. The vaccine was well tolerated and plasma HIV RNA levels remained undetectable after each immunization time point (0, 4, and 12 weeks). The inventors demonstrate a significant increase in CD4+ T cell counts in vaccine recipients compared to the control individuals after 12 months. Individuals with evidence of HIV-specific responses demonstrated the highest gain in CD4+ T cell counts following three immunizations of LFn-p24C.

Following the twelve-month safety evaluation process, the volunteers were asked to undergo a 30-day period of observed treatment interruption of conventional anti-retroviral treatment. Eight out of twenty-four (30%) individuals showed no evidence of viral rebound during treatment interruption. All volunteers demonstrated prompt suppression of viral load following resumption of ART. The inventors therefore demonstrate the safety and efficacy of an HIV vaccine in infected Ugandans and that adjunct therapeutic immunization may benefit select individuals in further boosting the immune response.

Without wishing to be bound by theory, effective conventional, many anti-retroviral treatments, even multiple drug treatments for HIV require strict compliance with a complex treatment regimen that can require the administration of many different drugs per day, administered at a precisely timed intervals, and with careful attention to diet. Patient non-compliance is a well-known problem accompanying such complex treatment regimens in the treatment of HIV because such non-compliance may lead to the emergence of multiple drug resistant strains of HIV and also abandonment of treatment in the middle of the therapy.

As demonstrated herein, a composition comprising a HIV polypeptide and LFn, (e.g., as a fusion protein or using a non-covalent attachment), can be used in combination with conventional anti-retroviral therapy to increase the efficacy of the conventional anti-retroviral therapy. In particular, in some embodiments, pulsed administration of the HIV-LFn vaccine composition allows for breaks or interruptions in continuous conventional anti-retroviral treatment. Each pulse dose of the HIV-LFn vaccine composition as disclosed herein can be used to reduce the total amount of anti-retroviral treatment over the course of treatment, as breaks in the continuous conventional anti-retroviral treatment are allowed. In fact, the inventors surprisingly discovered that administration of a HIV-LFn vaccine composition allowed for unintended breaks in a continuous conventional anti-retroviral treatment without significantly increasing the viral load during the break from the anti-retroviral treatment.

Accordingly, one aspect relates to methods to a composition comprising a HIV polypeptide and LFn (e.g., as a fusion protein or using a non-covalent attachment), as a vaccine for allowing a greater flexibility of the daily regimes. Such a composition is particularly useful in countries where it may be difficult to rigorously follow a specific antiretroviral drug regimen.

Accordingly, the present invention relates to the use of a vaccine composition comprises a LFn polypeptide complexed with a HIV antigen (e.g., as a fusion protein or other non-covalent bond association) in combination with traditional retroviral therapy or combination HIV viral therapy. Accordingly, the present invention relates to a dual therapeutic approach using vaccines on a periodic basis (e.g., pulsed administration), in combination with traditional combination retroviral therapy to enhance the efficacy of the traditional retroviral therapy in subjects positive for HIV or suffering from AIDS.

In one embodiment, the vaccine composition as disclosed herein comprises a LFn polypeptide and a HIV antigen allows subjects to take periodic breaks from the traditional combination retroviral therapy, including untentional breaks which is a frequent problem with HIV anti-retroviral therapy (referred herein as “ART”). In some embodiments, where a subject has been administered a vaccine composition comprising a LFn polypeptide and a HIV antigen, the subject can withdrawal from taking the take traditional antiviral drugs for a limited period of time, for example, at least a 1 week break, or about a 2 week break or about a 3 week break or a month break or longer from the conventional antiretroviral regimen. Thus, the present invention affords subjects the flexibility to withdrawal from taking traditional antiviral drugs and flexibility in need to be compliant with the stringent drug antiviral therapy regimens without the risk of decreasing efficacy of the traditional antiviral drugs.

In some embodiments, a subject is administered a pharmaceutical vaccination composition comprising LFn polypeptide and a HIV antigen (e.g., as a fusion protein or using a non-covalent attachment) periodically, e.g., in pulsed intervals, for example, once a month, or once every other month, or quarterly, or twice a year, or once a year.

### DESCRIPTION OF THE DRAWINGS

FIGS. 1A-B show local and systemic reactogenicity following 3 immunizations and a booster dose, for Phase 1A and 1B, respectively. FIG. 1A shows the results of Phase 1A study and FIG. 1B shows the results of Phase 1B study. A total of 840 events were recorded. 24/840 (2.9%) of the AE's were documented as mild and 1/840 (0.1%) were recorded as moderate in severity. There were no severe adverse events deemed related to the study vaccine.

FIG. 2 shows CD4 count distribution in phase 1A for historical control individuals and vaccine recipients (dashed and clear boxplots, respectively). Horizontal lines represent medians and interquartile range (25th and 75th percentiles). No statistical significance differences in CD4+ T cell distribution in the control group was observed (12 months, p=0.41) or in vaccine recipients (6 months prior to enrollment, p=0.2). Significant increase in CD4 cell count was observed after three immunizations at 12 months and after 15 months (p=0.02 and 0.006, respectively).

FIGS. 3A-3B show CD4 and CD8 immune responses to vaccine recipients. FIG. 3A shows immune Activation. PBMC were stained with HLADR FITC, CD38 PE, CD3 AmCyan, CD8 PerCPCy5.5, CD4 APC Cy7 and analyzed by flow cytometry. Samples were first gated on the CD3⁺/CD8⁺ and CD3⁺CD4⁺ lymphocyte populations, and the percent of CD38- and HLADR-positive cells were determined. No significant differences were observed in immune activation in CD4⁺/CD8⁺ T-cell subpopulations between vaccine or control samples (p>0.5). FIG. 3B shows immune dysfunction was measured by PD-1 expression. PBMC were stained with CD3 AmCyan, CD8 PerCPCy5.5, CD4 APC Cy7 and PD-1 APC. Samples were first gated on the CD3⁺/CD4⁺ (and CD3⁺/CD8⁺) lymphocyte population, and the percent of PD-1-positive cells was subsequently determined. CD4+PD1+ and CD8+PD1+ expression was significantly higher in control compared to vaccine samples (p=0.016 and 0.041), respectively. Horizontal lines represent medians and interquartile range (25th and 75th percentiles).

FIG. 4A-4B shows proliferation of CD4 and CD8 cells after stimulation with Gag peptides. FIG. 4A shows a representative plot of Gag-specific CD4 proliferation. CFSE-labeled PBMC were stimulated with subtype C Gag peptides for 5 days then assessed for proliferation by flow cytometry. Results are expressed as the percent of proliferating CD4+ T-cells as measured by the extent of CFSE dilution. Positive proliferation is defined as >0.1% net and at least twice background. FIG. 4B shows Gag-specific. FIG. 4C shows CMV-specific CD4+ and CD8+ proliferation in vaccinees and control samples. A significant difference between the frequency of responses in CD4- and CD8-mediated proliferation was observed between control and vaccine recipients for Gag (p<0.05) but not CMV (p>0.05)

FIG. 5 shows a box-plot of the correlation between vaccine specific T-cell proliferation and CD4 count increase. CD4+ T cell profile of Phase 1A vaccine recipients with (+) and without (−) evidence of vaccine-specific cell proliferation. The mean CD4 gain in the (+) group was 151 compared to 36 (−) in the non-immunized group. Horizontal lines represent mean values.

FIGS. 6A-6B show immunological and virological characteristics of Phase 113 vaccine recipients. Twenty-four individuals discontinued ART for a period of 4 weeks after receiving a booster of LFn-p24C. FIG. 6A shows Viral load (HIV RNA copies/ml plasma) and absolute numbers of CD4⁺ T cells per mm³, FIG. 6B shows blood were monitored throughout treatment interruption and cessation periods. Blue shading depicts periods off ART.

FIG. 7 shows a box-plot of the association between therapeutic immunization and expression of programmed death 1 (PD-1)

FIG. 8 shows that 33% (8/24) of the therapeutic vaccines showed complete viral load suppression during scheduled treatment interruption.

FIG. 9 shows that 16% (4/24) of the therapeutic vaccines showed low viral load rebound during scheduled treatment interruption.

FIG. 10 shows that a total 50% of the therapeutic vaccines showed suppressed viral load during scheduled treatment interruption.

FIG. 11 shows that 50% of the therapeutic vaccines had viral load rebound during scheduled treatment interruption; no drug resistant viruses appeared.

### DETAILED DESCRIPTION OF THE INVENTION

The inventors have demonstrated that a HIV polypeptide conjugated to LFn can be used in combination with conventional HIV anti-retroviral therapy for increased efficacy of the conventional retroviral therapy. Accordingly, one aspect of the present invention relates to use of a vaccine composition comprising a HIV antigen (e.g., polypeptide or peptide) and LFn, (e.g., as a fusion protein or using a non-covalent attachment) to allow interruptions or breaks in the continuous administration of conventional HIV anti-retroviral drugs. In some embodiments, the LFn and HIV antigen are a LFn-HIV antigen fusion protein, and in alternative embodiments, the LFn and HIV antigen are associated using a non-covalent attachment.

Accordingly, one aspect relates to methods to use of a vaccine composition comprising a HIV polypeptide conjugated to LFn, for allowing a greater flexibility of the daily regimes in countries where it may be difficult to rigorously follow a specific antiretroviral drug regimen. This represents a significant saving in time, effort and expense and, more importantly, will maintain efficacy of conventional HIV anti-retroviral drugs for a longer period of time should a patient inadvertently or deliberately fail to follow the strict antiretroviral drug regimen.

Accordingly, the present invention relates to the use of a vaccine composition comprising a LFn polypeptide and a HIV antigen in combination with traditional retroviral therapy or combination HIV viral therapy. Accordingly, the present invention relates to a dual therapeutic approach using both vaccines on a periodic basis (e.g., pulsed administration), in combination with traditional retroviral therapy to enhance the efficacy of the traditional retroviral therapy in subjects positive for HIV or suffering from AIDS.

The effective conventional, even multiple drug treatments for HIV often require strict compliance with a complex treatment regimen that can require the administration of many different drugs per day, administered at a precisely timed intervals with careful attention to diet. Patient non-compliance is a well-known problem accompanying such complex treatment regimens in the treatment of HIV because such non-compliance may lead to the emergence of multiple drug resistant strains of HIV and also abandonment of treatment in the middle of the therapy.

In one embodiment, a vaccine composition as disclosed herein comprising a LFn polypeptide and a HIV antigen allows a subject to take periodic breaks from the traditional continuous retroviral therapy. In some embodiments, where a subject has been administered a vaccination if a LFn polypeptide fused to a HIV antigen, the subject can withdrawal from taking the take traditional antiviral drugs for a limited period of time, for example, at least a 1 week break, or about a 2 week break or about a 3 week break or a month break or longer from the conventional antiretroviral regimen. Thus, the present invention affords subjects the flexibility to withdrawal from taking traditional antiviral drugs and flexibility in need to be compliant with the stringent drug antiviral therapy regimens without the risk of decreasing efficacy of the traditional antiviral drugs.

In some embodiments, a subject is administered a pharmaceutical vaccination composition comprising LFn polypeptide fused to a HIV antigen periodically, for example, once a month, or once every other month, or quarterly, or twice a year, or once a year.

Accordingly, the present invention allows the therapeutic vaccine comprising LFn polypeptide fused to a HIV antigen as a combination therapy to reduce the increase efficacy of conventional HIV drugs and importantly, simplifies dosing schedule thereby increases patient compliance. In some embodiments, the combination of the vaccine comprising LFn polypeptide fused to a HIV antigen also increases the drug efficacy of conventional HIV therapies. In some embodiments, the use of the vaccine comprising a LFn polypeptide and a HIV antigen in combination with traditional HIV anti-retroviral therapies can yield an equivalent antiviral effect with reduced toxicity. This is particularly useful for the development of a combination for acute therapy and/or for resistant HIV viruses.

In some embodiments, one object of the present invention is to provide a pharmaceutical vaccine composition comprising LFn polypeptide and a HIV antigen for use in treating individuals having the human immunodeficiency virus (HIV), and optionally related disorders resulting in AIDS.

### DEFINITIONS OF TERMS

The term “vaccine composition” used herein is defined as composition used to elicit an immune response against an antigen within the composition in order to protect or treat an organism against disease.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention.

As used herein, the term “fused” means that one protein is physically associated with a second protein, for example via an electrostatic or hydrophobic interaction or a covalent linkage. Covalent linkage can encompass linkage as a fusion protein or chemically coupled linkage, for example via a cysteine residue.

As used herein, the term “fusion polypeptide” or “fusion protein” means a protein created by joining two polypeptide coding sequences together. The fusion polypeptides of this invention are fusion polypeptides formed by joining a coding sequence of a LF polypeptide or fragment or mutant thereof with a coding sequence of a second polypeptide to form a fusion or chimeric coding sequence such that they constitute a single open-reading frame. The fusion coding sequence, when transcribed and translated, expresses a fusion polypeptide. In other words, a “fusion polypeptide” or “fusion protein” is a recombinant protein of two or more proteins which are joined by a peptide bond.

As used herein, the term “protein” and “polypeptide” are used interchangeably.

As used herein, the term “promotes transmembrane delivery” refers to the ability of a first polypeptide to facilitate a second protein to traverse the membrane of an intact, living cell.

As used herein, the term “cytosol” refers to the interior of an intact cell. The “cytosol” comprises the cytoplasm and the organelles inside a cell.

As used herein, the term “an intact cell” refers to a living cell with an unbroken, uncompromised plasma membrane, which cell has a differential membrane potential across the membrane, with the inside of the cell being negative with respect to the outside of the cell.

As used herein, the term “N-glycosylated” or “N-glycosylation” refers to the covalent attachment of a sugar moiety to asparagine residues in a polypeptide. Sugar moieties can include but are not limited to glucose, mannose, and N-acetylglucosamine. Modifications of the glycans are also included, e.g. siaylation. The LFn polypeptide has three N-glycosylation sites: asparagine positions 62, 212, and 286 in the 809 amino acid polypeptide.

As used herein, the terms “N-glycosylated LFn-fusion polypeptide,” “N-glycosylated LF-fusion polypeptide” or “N-glycosylated fused polypeptide” refer to a fusion polypeptide, as defined herein, that has at least one sugar moiety covalently attached to an asparagine residue. For example, Asn-62, Asn-212, and Asn-286 can be glycosylated in an N-glycosylated LF-fusion polypeptide.

As used herein, the term “substantially lacks amino acids 1-33” in the context of a fusion polypeptide described herein refers to a fusion polypeptide that lacks signal peptide activity.

As used herein, the term “antigen” refers to any substance that prompts an immune response directed against the substance.

An antigen presenting cell is a cell that expresses the Major Histocompatibility complex (MHC) molecules and can display foreign antigen complexed with MHC on its surface. Examples of antigen presenting cells are dendritic cells, macrophages, B cells, fibroblasts (skin), thymic epithelial cells, thyroid epithelial cells, glial cells (brain), pancreatic beta cells, and vascular endothelial cells.

The term “lethal factor” or “LF” as used herein refers generally to a non-PA polypeptide of the bipartite B. anthracis exotoxin. Wild-type, intact B. anthracis LF polypeptide has the amino acid sequence set out in GenBank Accession Number M29081 (Gene ID No: 143143), which corresponds to SEQ ID NO: 1. SEQ ID NO: 1 corresponds to LF with a signal peptide located at residues 1 to 33 at its N-terminus. Stated another way, immature wild-type LF corresponds to an 809 amino acid protein, which includes a 33 amino acid signal peptide at the N-terminus. The amino acid sequence of immature wild-type LF (SEQ ID NO: 1) with the signal peptide highlighted in bold is as follows:

(SEQ ID NO: 1)
MNIKKEFIKVISMSCLVTAITLSGPVFIPLVQGAGGHGDVGMHVKEKEKNKDENKRKDEERNK
TQEEHLKEIMKHIVKIEVKGEEAVKKEAAEKLLEKVPSDVLEMYKAIGGKIYIVDGDITKHIS
LEALSEDKKKIKDIYGKDALLHEHYVYAKEGYEPVLVIQSSEDYVENTEKALNVYYEIGKILS
RDILSKINQPYQKFLDVLNTIKNASDSDGQDLLFTNQLKEHPTDFSVEFLEQNSNEVQEVFAK
AFAYYIEPQHRDVLQLYAPEAFNYMDKFNEQEINLSLEELKDQRMLSRYEKWEKIKQHYQHWS
DSLSEEGRGLLKKLQIPIEPKKDDIIHSLSQEEKELLKRIQIDSSDFLSTEEKEFLKKLQIDI
RDSLSEEEKELLNRIQVDSSNPLSEKEKEFLKKLKLDIQPYDINQRLQDTGGLIDSPSINLDV
RKQYKRDIQNIDALLHQSIGSTLYNKIYLYENMNINNLTATLGADLVDSTDNTKINRGIFNEF
KKNFKYSISSNYMIVDINERPALDNERLKWRIQLSPDTRAGYLENGKLILQRNIGLEIKDVQI
IKQSEKEYIRIDAKVVPKSKIDTKIQEAQLNINQEWNKALGLPKYTKLITFNVHNRYASNIVE
SAYLILNEWKNNIQSDLIKKVTNYLVDGNGRFVFTDITLPNIAEQYTHQDEIYEQVHSKGLYV
PESRSILLHGPSKGVELRNDSEGFIHEFGHAVDDYAGYLLDKNQSDLVTNSKKFIDIFKEEGS
NLTSYGRTNEAEFFAEAFRLMHSTDHAERLKVQKNAPKTFQFINDQIKFIINS

Cleavage of the immature LF protein results in a mature wild-type LF polypeptide of 776 amino acids in length. The 776 amino acid polypeptide sequence of mature wild-type LF polypeptide (i.e. lacking the N-terminal signal peptide) corresponds to SEQ ID NO: 2, as follows:

(SEQ ID NO: 2)
AGGHGDVGMHVKEKEKNKDENKRKDEERNKTQEEHLKEIMKHIVKIEVKGEEAVKKEAAEKLL
EKVPSDVLEMYKAIGGKIYIVDGDITKHISLEALSEDKKKIKDIYGKDALLHEHYVYAKEGYE
PVLVIQSSEDYVENTEKALNVYYEIGKILSRDILSKINQPYQKFLDVLNTIKNASDSDGQDLL
FTNQLKEHPTDFSVEFLEQNSNEVQEVFAKAFAYYIEPQHRDVLQLYAPEAFNYMDKFNEQEI
NLSLEELKDQRMLSRYEKWEKIKQHYQHWSDSLSEEGRGLLKKLQIPIEPKKDDIIHSLSQEE
KELLKRIQIDSSDFLSTEEKEFLKKLQIDIRDSLSEEEKELLNRIQVDSSNPLSEKEKEFLKK
LKLDIQPYDINQRLQDTGGLIDSPSINLDVRKQYKRDIQNIDALLHQSIGSTLYNKIYLYENM
NINNLTATLGADLVDSTDNTKINRGIFNEFKKNFKYSISSNYMIVDINERPALDNERLKWRIQ
LSPDTRAGYLENGKLILQRNIGLEIKDVQIIKQSEKEYIRIDAKVVPKSKIDTKIQEAQLNIN
QEWNKALGLPKYTKLITFNVHNRYASNIVESAYLILNEWKNNIQSDLIKKVTNYLVDGNGRFV
FTDITLPNIAEQYTHQDEIYEQVHSKGLYVPESRSILLHGPSKGVELRNDSEGFIHEFGHAVD
DYAGYLLDKNQSDLVTNSKKFIDIFKEEGSNLTSYGRTNEAEFFAEAFRLMHSTDHAERLKVQ
KNAPKTFQFINDQIKFIINS.

The term “LF polypeptide” applies not only to full-length, wild-type LF (with or without the signal sequence), but also to fragments thereof that mediate intracellular delivery of fused or physically associated polypeptides to an intact cell, such as, an antigen presenting cell. Also included in the term “LF polypeptide” are conservative substitution variants of LF, including conservative substitution variants that mediate such intracellular delivery.

The term “LFn polypeptide” refers to an N-terminal fragment of B. anthracis LF that does not display zinc metalloproteinase activity and does not inactivate mitogen-activated kinase activity, yet does mediate intracellular or transmembrane delivery of fused polypeptides. LFn polypeptides as defined and described herein are preferred. In one aspect, “LFn polypeptide” includes SEQ ID NO: 3, which corresponds to a 288 amino acid immature LFn protein; this LFn protein is “immature” in that it includes a signal peptide located at residues 1 to 33 of the N-terminus. Stated another way, immature LFn corresponds to a 288 amino acid protein, which includes a 33 amino acid signal peptide at the N-terminus. Cleavage of the immature LFn protein of SEQ ID NO: 3 results in a mature LFn polypeptide of 255 amino acids in length. It should be emphasized that, for the purposes of the methods and compositions described herein, the LF and/or LFn polypeptides can either include or lack the signal peptide—that is, the presence or absence of the signal peptide is not expected to influence the activity of LF polypeptides as transmembrane transport facilitators in the methods described herein. The amino acid sequence of immature LFn (SEQ ID NO: 3) with the signal peptide highlighted in bold is as follows:

(SEQ ID NO: 3)
MNIKKEFIKVISMSCLVTAITLSGPVFIPLVQGAGGHGDVGMHVKEKEKNKDENKRKDEERNK
TQEEHLKEIMKHIVKIEVKGEEAVKKEAAEKLLEKVPSDVLEMYKAIGGKIYIVDGDITKHIS
LEALSEDKKKIKDIYGKDALLHEHYVYAKEGYEPVLVIQSSEDYVENTEKALNVYYEIGKILS
RDILSKINQPYQKFLDVLNTIKNASDSDGQDLLFTNQLKEHPTDFSVEFLEQNSNEVQEVFAK
AFAYYIEPQHRDVLQLYAPEAFNYMDKFNEQEINLS

The polypeptide sequence of a mature LFn polypeptide (which lacks the N-terminal signal peptide) is 255 amino acids in length and corresponds to SEQ ID NO: 4 is as follows:

(SEQ ID NO: 4)
AGGHGDVGMHVKEKEKNKDENKRKDEERNKTQEEHLKEIMKHIVKIEVKGEEAVKKEAAEKLL
EKVPSDVLEMYKAIGGKIYIVDGDITKHISLEALSEDKKKIKDIYGKDALLHEHYVYAKEGYE
PVLVIQSSEDYVENTEKALNVYYEIGKILSRDILSKINQPYQKFLDVLNTIKNASDSDGQDLL
FTNQLKEHPTDFSVEFLEQNSNEVQEVFAKAFAYYIEPQHRDVLQLYAPEAFNYMDKFNEQEI
NLS

The term “functional fragment” as used in the context of a “functional fragment of LFn” refers to a fragment of an LFn polypeptide that mediates, effects or facilitates transport of an antigen across an intact, living cell's membrane. One example of such a fragment of an LFn polypeptide is a 104 amino acid C-terminal fragment of LFn corresponding to SEQ ID NO: 5 as follows (this sequence is also disclosed as SEQ ID NO: 3 in U.S. patent application Ser. No. 10/473,190, which is incorporated herein by reference):

(SEQ ID NO: 5)
GKILSRDILSKINQPYQKFLDVLNTIKNASDSDGQDLLFTNQLKEHPTDFSVEFLEQNSNEV
QEVFAKAFAYYIEPQHRDVLQLYAPEAFNYMDKFNEQEINLS

The term “LFn polypeptide” as used herein encompasses each of the “immature” LFn and “mature” LFn nolecules described herein, as well as fragments, variants (including conservative substitution variants) and derivatives thereof that mediate, effect or facilitate transport of a physically associated, e.g., fused, polypeptide across the membrane of an intact, living cell. Additional fragments of LFn polypeptides specifically contemplated for use in the methods, compositions and kits described herein include a fragment comprising, or optionally, consisting essentially of the C-terminal 60, 80, 90, 100 or 104 amino acids of SEQ ID NO: 3 or a conservative substitution variant thereof that mediates, effects or facilitates transfer of a physically associated, e.g., fused polypeptide across an intact membrane of a living cell.

The term “adjuvant” as used herein refers to any agent or entity which increases the antigenic response or immune response by a cell to a HIV antigen. Examples of adjuvants include, but are not limited to mineral gels such as aluminum hydroxide; surface active substances such as lysolecithin, pluronic polyols, polyanions; other peptides; oil emulsions; and potentially useful human adjuvants such as BCG and Corynebacterium parvum. QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59.

The terms “protective antigen” or “PA” (when used relative to B. anthracis) are used interchangeably herein to refer to part of the B. anthracis exotoxin bipartite protein which binds to a mammalian cell's surface by cellular receptors. A “PA,” as the term is used in this context has its receptor binding site intact and functional. U.S. Pat. Nos. 5,591,631 and 5,677,274 (incorporated by reference in their entirety) describe PA fusion proteins that target PA to particular cells, such as cancer cells and HIV-infected cells, using as fusion partners ligands for receptors on the targeted cells.

A “fragment” of a HIV antigen as that term is used herein will be at least 6 amino acids in length, and can be, for example, at least 8, at least 10, at least 14, at least 16, at least 17, at least 18, at least 19, at least 20 or at least 25 amino acids or greater.

The terms “Cytotoxic T Lymphocyte” or “CTL” refers to lymphocytes which induce apoptosis in targeted cells. CTLs form antigen-specific conjugates with target cells via interaction of TCRs with processed antigen (Ag) on target cell surfaces, resulting in apoptosis of the targeted cell. Apoptotic bodies are eliminated by macrophages. The term “CTL response” is used to refer to the primary immune response mediated by CTL cells.

The term “cell mediated immunity” or “CMI” as used herein refers to an immune response that does not involve antibodies or complement but rather involves the activation of, for example, macrophages, natural killer cells (NK), antigen-specific cytotoxic T-lymphocytes (T-cells), and the release of various cytokines in response to a HIV antigen. Stated another way, CMI refers to immune cells (such as T cells and lymphocytes) which bind to the surface of other cells that display a target antigen (such as antigen presenting cells (APS)) and trigger a response. The response may involve either other lymphocytes and/or any of the other white blood cells (leukocytes) and the release of cytokines. Cellular immunity protects the body by: (i) activating antigen-specific cytotoxic T-lymphocytes (CTLs) that are able to destroy body cells displaying epitopes of foreign antigen on their surface, such as virus-infected cells and cells with intracellular bacteria; (2) activating macrophages and NK cells, enabling them to destroy intracellular pathogens; and (3) stimulating cells to secrete a variety of cytokines that influence the function of other cells involved in adaptive immune responses and innate immune responses.

The term “immune cell” as used herein refers to any cell which can release a cytokine in response to a direct or indirect antigenic stimulation. Included in the term “immune cells” herein are lympocytes, including natural killer (NK) cells, T-cells (CD4+ and/or CD8+ cells), B-cells, macrophages and monocytes, Th cells; Th1 cells; Th2 cells; Tc cells; leukocytes; dendritic cells; macrophages; mast cells and monocytes and any other cell which is capable of producing a cytokine molecule in response to direct or indirect antigen stimulation. Typically, an immune cell is a lymphocyte, for example a T-cell lymphocyte.

The term “cytokine” as used herein is used interchangeably with the term “effector molecule,” and refers to a molecule released from an immune cell in response to stimulation with an antigen. Examples of such cytokines include, but are not limited to: GM-CSF; IL-1α; IL-1β; IL-2; IL-3; IL-4; IL-5; IL-6; IL-7; IL-8; IL-10; IL-12; IFN-α; IFN-β; IFN-γ; MIP-1α; MIP-1β; TGF-β; TNFα and TNFβ. The term “cytokine” does not include antibodies.

The term “complex” as used herein refers to a collection of two or more molecules, connected spatially by means other than a covalent interaction; for example they can be connected by electrostatic interactions such as van der Waals forces etc.

The term “translocated into a cell” refers to the movement of a moiety, such as a HIV antigen, and optionally a fusion polypeptide described herein from a location outside a cell, across the plasma membrane to the inside of an intact, living cell.

The term “in vivo” refers to assays or processes that occur in an animal.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. In some embodiments, a mammal is a human.

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like.

The terms “polypeptide” and “protein” are used interchangeably to refer to a polymer of amino acid residues linked by peptide bonds, and for the purposes of the claimed invention, have a minimum length of at least 15 amino acids. Oligopeptides, oligomers multimers, and the like, typically refer to longer chains of amino acids and are also composed of linearly arranged amino acids linked by peptide bonds, whether produced biologically, recombinantly, or synthetically and whether composed of naturally occurring or non-naturally occurring amino acids, are included within this definition. Both full-length proteins and fragments thereof greater than 15 amino acids are encompassed by the definition. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. For the methods and compositions described herein, the term “peptide” refers to a sequence of peptide bond-linked amino acids containing between 6 amino acids and 15 amino acids in length.

It will be appreciated that proteins or polypeptides often contain amino acids other than the 20 amino acids commonly referred to as the 20 naturally occurring amino acids, and that many amino acids, including the terminal amino acids, can be modified in a given polypeptide, either by natural processes such as glycosylation and other post-translational modifications, or by chemical modification techniques which are well known in the art. Known modifications which can be present in polypeptides of the present invention include, but are not limited to, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formulation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

As used herein, the terms “homologous” or “homologues” are used interchangeably, and when used to describe a polynucleotide or polypeptide, indicate that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides, usually from about 75% to 99%, and more preferably at least about 98 to 99% of the nucleotides. For a polypeptide, there should be at least 50% of amino acid identity in the polypeptide. The term “homolog” or “homologous” as used herein also refers to homology with respect to structure. Determination of homologs of genes or polypeptides can be easily ascertained by the skilled artisan. When in the context with a defined percentage, the defined percentage homology means at least that percentage of amino acid similarity. For example, 85% homology refers to at least 85% of amino acid similarity.

As used herein, the term “heterologous” reference to nucleic acid sequences, proteins or polypeptides mean that these molecules are not naturally occurring in that cell. For example, the nucleic acid sequence coding for a fusion LFn-HIV antigen polypeptide described herein that is inserted into a cell, e.g. in the context of a protein expression vector, is a heterologous nucleic acid sequence.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Where necessary or desired, optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

The term “variant” as used herein refers to a polypeptide or nucleic acid that differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Substitutions encompassed by variants as described herein may also be “non conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). A “variant” of an LFn polypeptide refers to a molecule substantially similar in structure and function to that of a polypeptide of SEQ ID NO: 3, where the function is the ability to mediate, effect or facilitate transport of an associated or fused polypeptide across a cell membrane of a living cell from a subject. In some embodiments, a variant of SEQ ID NO: 3 or SEQ ID NO: 4 is a fragment of SEQ ID NO: 3 or 4 as disclosed herein, such as SEQ ID NO: 5.

The term “substantially similar,” when used in reference to a variant of LFn or a functional derivative of LFn as compared to the LFn protein encoded by SEQ ID NO: 3 means that a particular subject sequence, for example, an LFn fragment or LFn variant or LFn derivative sequence, varies from the sequence of the LFn polypeptide encoded by SEQ ID NO: 3 by one or more substitutions, deletions, or additions relative to SEQ ID NO: 3, but retains at least 50% of the transmembrane transport facilitation activity, and preferably higher, e.g., at least 60%, 70%, 80%, 90% or more exhibited by the LFn protein of SEQ ID NO: 3. (It is acknowledged that LFn does not occur naturally—reference to a “native” or “natural” LFn sequence is intended to convey that the sequence is identical to the portion of naturally-occurring LF polypeptide designated as LFn herein.) In determining polynucleotide sequences, all subject polynucleotide sequences capable of encoding substantially similar amino acid sequences are considered to be substantially similar to a reference polynucleotide sequence, regardless of differences in codon sequence. A nucleotide sequence is “substantially similar” to a given LFn nucleic acid sequence if: (a) the nucleotide sequence hybridizes to the coding regions of the native LFn sequence, or (b) the nucleotide sequence is capable of hybridization to nucleotide sequence of LFn encoded by SEQ ID NO: 1 under moderately stringent conditions and has biological activity similar to the native LFn protein; or (c) the nucleotide sequences are degenerate as a result of the genetic code relative to the nucleotide sequences defined in (a) or (b). Substantially similar proteins will typically be greater than about 80% similar to the corresponding sequence of the native protein.

Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. “Conservative amino acid substitutions” result from replacing one amino acid with another having similar structural and/or chemical properties. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W.H. Freeman and Company (1984).)

The choice of conservative amino acids may be selected based on the location of the amino acid to be substituted in the peptide, for example if the amino acid is on the exterior of the peptide and exposed to solvents, or on the interior and not exposed to solvents. Selection of such conservative amino acid substitutions is within the skill of one of ordinary skill in the art and is described, for example by Dordo et al., J. Mol. Biol, 1999, 217, 721-739 and Taylor et al., J. Theor. Biol. 119 (1986); 205-218 and S. French and B. Robson, J. Mol. Evol. 19 (1983)171. Accordingly, one can select conservative amino acid substitutions suitable for amino acids on the exterior of a protein or peptide (i.e. amino acids exposed to a solvent). These substitutions include, but are not limited to the following: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions suitable for amino acids on the interior of a protein or peptide (i.e. the amino acids are not exposed to a solvent). For example, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, LF polypeptides including non-conservative amino acid substitutions are also encompassed within the term “variants.” A variant of an LFn polypeptide, for example a variant of SEQ ID NO: 3 or 4 is meant to refer to any molecule substantially similar in structure (i.e., having at least 50% homology as determined by BLASTp analysis using default parameters) and function (i.e., at least 50% as effective as a polypeptide of SEQ ID NO: 3 in transmembrane transport) to a molecule of SEQ ID NO: 3 or 4.

As used herein, the term “non-conservative” refers to substituting an amino acid residue for a different amino acid residue that has different chemical properties. Non-limiting examples of non-conservative substitutions include aspartic acid (D) being replaced with glycine (G); asparagine (N) being replaced with lysine (K); and alanine (A) being replaced with arginine (R).

The term “derivative” as used herein refers to peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half life, etc. The moieties can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

The term “functional” when used in conjunction with “derivative” or “variant” refers to a protein molecule which possesses a biological activity that is substantially similar to a biological activity of the entity or molecule of which it is a derivative or variant. By “substantially similar” in this context is meant that the biological activity, e.g., transmembrane transport of associated polypeptides is at least 50% as active as a reference, e.g., a corresponding wild-type polypeptide, and preferably at least 60% as active, 70% as active, 80% as active, 90% as active, 95% as active, 100% as active or even higher (i.e., the variant or derivative has greater activity than the wild-type), e.g., 110% as active, 120% as active, or more.

The term “recombinant” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide, means a polypeptide produced by expression from a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “vectors” refers to a nucleic acid molecule capable of transporting or mediating expression of a heterologous nucleic acid to which it has been linked to a host cell; a plasmid is a species of the genus encompassed by the term “vector.” The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome. Preferred vectors are those capable of autonomous replication and/or expression of nucleic acids to which they are linked

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and the include plural referents unless context clearly indicates otherwise. Similarly, the word or is intended to include and unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### Vaccine Composition

One aspect of the present invention relates to a vaccine composition comprising a LFn polypeptide and at least one HIV antigen. In some embodiments, the LFn polypeptide and HIV antigen are covalently linked as a fusion protein. In one embodiment, the HIV antigenic polypeptide (e.g., HIV antigen) is conjugated to the LFn polypeptide or a fragment thereof. In some embodiments the cross-link can be a covalent bond (e.g., as a fusion protein), and in some embodiments, the cross-link can be, e.g., via free sulfhydryl group in the endodomain of the isolated whole HIV antigen. Methods of forming such conjugates are described in U.S. Pat. No. 5,612,037 which is incorporated herein in its entirety by reference.

In alternative embodiments, LFn and the HIV polypeptide antigen are non-covalently linked, e.g., the LF polypeptide can be in a non-covalently linked complex or be associated with the target antigen in some way, for example, to form an LFn:HIV antigen complex, where the LFn and HIV antigen are associated by forces other than a covalent bond, such as van der Waals forces, electrostatic forces and the like. In some embodiments of this and other aspects described herein, the composition comprises an LF polypeptide: HIV antigen complex, where the LF polypeptide, e.g., LFn, is directly associated with the target antigen by van der Waals forces or other non-covalent interactions. In alternative embodiments, the composition comprises an LF polypeptide: HIV antigen complex, where the LF polypeptide, e.g., an LFn polypeptide is indirectly associated with the HIV antigen, for example by interaction of the LFn polypeptide with at least a third entity or moiety, and the HIV antigen also interacts with a separate portion of the third entity (that interacts with the LF polypeptide).

In some embodiments of this and other aspects described herein, the composition comprises an LF polypeptide or LFn polypeptide and a HIV antigen, where the LF polypeptide is not covalently linked to the target antigen but the LF polypeptide is non-covalently associated or complexed with the target antigen in some way. For example, to form an LFn:HIV antigen complex. In some embodiments, the composition comprises an LFn:HIV antigen complex, where the LFn (or fragment or variant thereof) is directly associated with the target antigen by van der Waals forces or other non-covalent interactions. In alternative embodiments, the composition comprises an LFn:HIV antigen complex, where the LFn (or fragment or variant thereof) is indirectly associated with the target antigen, such as for example by interaction of the LFn (or fragment or variant thereof) with at least one third moiety, and the target antigen interacts with the same third moiety that interacts with the LFn polypeptide. Such interactions can be any non-covalent bond association known by a skilled artisan, such as, for example but not limited to, van der Waals forces, hydrophilic interactions, hydrophobic interactions and other non-covalent interactions. In some embodiments, at least one, or at least two, or at least 3, or at least 4 or more third entities can be used to associate LFn (or a fragment or variant thereof) with the HIV antigen. For example, the present invention comprises compositions which comprise complexes such as, an LFn:moiety:HIV antigen complex, or Lfn:moiety:moiety:HIV antigen complex, Lfn:moiety:moiety:moiety:HIV antigen complex, and such like complexes. In some embodiments, a moiety which associates with LFn can be the same or different from a moiety which binds with the HIV antigen, and all the moieties can be the same within a complex, or different within the complex.

### HIV Antigens

It is also contemplated that the vaccine composition as described herein can comprise one or more of a plurality of HIV antigenic polypeptides described herein. Preferably, the vaccine composition comprises at least LFn and at least one HIV antigenic polypeptides, e.g., p24, gag or other HIV polypeptides, together in any combination, or separately fused to a LFn polypeptide. Any HIV polypeptide can be used which are commonly known to persons of ordinary skill In the art and include, for example, but are not limited to peptides for HIV antigens as vaccines are disclosed in U.S. Pat. Nos. 7,067,134 and 7,067,134 which are incorporated herein in their entirety by reference. In some embodiments, a HIV antigen used in the vaccine composition as disclosed herein can be from any retrovirus including HIV-1, HIV-2, SIV, HTLV-1. In some embodiments, an HIV antigen is a human immunodeficiency virus polypeptide selected from HIV-1 and HIV-2, more preferably, the retrovirus is HIV-1. In some embodiments, HIV antigenic polypeptides can be components from different clades of Env (optionally Env chimeras) and Gag-Pol-(optionally) Nef from a single Glade, as disclosed in U.S. Applications 2008/0286306 and 2009/0227658, which are incorporated herein in their entirety by reference.

In some embodiments, the HIV antigen is an envelope protein, and can be selected from any of gp41, gp120, gp160 or a fragment thereof. Other HIV proteins can be used as HIV antigens in the vaccine composition as disclosed herein, e.g., such other HIV proteins include, but are not limited to, gag polypeptide, POL, protease, Nef, Vpr, Vpu, Tat 1, Tat2, reverse transcriptase, integrase, Vif, etc.

In one embodiment, a HIV antigen polypeptide is folded in its native conformation. In one embodiment, a HIV antigen polypeptide is part of a multi-molecular polypeptide complex. In one embodiment, a HIV antigen polypeptide is a subunit polypeptide of a multi-molecular polypeptide target antigen.

In some embodiments, a HIV antigen can be an intact (i.e. an entire or whole or complete) HIV antigen which is delivered to the cytosol of a cell by a non-linked or non-covalently linked LF polypeptide as described herein. By “intact” in this context is meant that the HIV antigen is the full length target antigen as that antigen polypeptide occurs in nature. This is in direct contrast to delivery of only a small portion or peptide of the target antigen. By delivering an intact HIV antigen to a cell, the LFn polypeptide enables or facilitates the translocation of the whole HIV antigen across the cell membrane and the display of a full range of epitopes of the intact target antigen in complexes with MHC I molecules. Moreover, this also facilitates detection of a cell mediated immune (CMI) response to a full range of epitopes of the intact target antigen, rather than just a single or selected few peptide epitopes. CMI occurs when T cells (lymphocytes) bind to the surface of other cells that display the antigen and trigger a response, e.g. production and release of cytokines. The response can involve other lymphocytes and any of the other white blood cells (leukocytes).

Accordingly, the vaccine composition comprising a HIV antigen and an LFn polypeptide (which is non-linked or non-covalently linked to the intact HIV antigen) can be used for a stronger and more robust CMI response to the intact target antigen as compared to use of the intact target antigen alone or to a part (i.e. a peptide) of the target antigen, as a CMI response can be raised against essentially any epitope of the whole antigen.

In some embodiments, an intact HIV antigen can be divided into fragments, or parts, of the whole HIV antigen, for example, at least two, or at least 3, or at least 4, or a least 5 or more HIV antigen fragments, depending on size of the intact HIV antigen protein. These fragments of the whole HIV antigen can be used, for example, as a quality control to filter out false positives of a positive CMI response. By way of an example only, a positive CMI response to a whole HIV antigen can be confirmed by assessing a CMI response to a panel of HIV antigens which are fragments of the whole HIV antigen. A true CMI response is confirmed if one or two of the fragments give a positive response, but not all fragments. If a positive CMI response is detected for all fragments, it is likely that the positive CMI response was a false positive.

In some embodiments, an intact HIV antigen can be divided into many parts, depending on the size of the initial HIV antigen, for use as a panel of sub-HIV antigens. Typically, where a whole HIV antigen is a multimer polypeptide, the whole HIV protein can be divided into sub-units and/or domains which can each individually can be mixed with an LF polypeptide and used in assay methods and compositions as disclosed herein. Alternatively, an intact HIV antigen can be divided into fragments, or parts of the whole HIV antigen, for example, at least two, or at least 3, or at least 4, or a least 5, or at least 6, or at least 7, or at least 8, or at least about 9, or at least about 10, or at least about 11, or at least about 12, or at least about 13, or at least about 15, or at least about 20, or at least about 25, or more than 25 fragments, and each fragment, individually or in combination, mixed with an LF polypeptide for use in assay methods and compositions as disclosed herein.

The fragmentation or division of a full length HIV antigen polypeptide can be an equal division of the full length HIV antigen polypeptide, or alternatively, in some embodiments, the fragmentation is asymmetrical or unequal. As a non-limiting example, where a HIV antigen is divided into two overlapping fragments, a HIV antigen can be divided into fragments of approximately the same (equal) size, or alternatively one fragment can be about 45% of the whole HIV antigen and the other fragment can be about 65%. As further non-limiting examples, a whole HIV antigen can be divided into a combination of differently sized fragments, for example, where a HIV antigen is divided into two fragments, fragments can be divided into about 40% and about 70%, or about 45% and about 65%; or about 35% and about 75%; or about 25% and about 85% of the whole HIV antigen. Any combination of overlapping fragments of a full length whole HIV antigen is encompassed for use in the generation of a panel of HIV antigens. Multiple HIV antigens can be combined, for example, the HIV env, gag and Pol to form empty HIV caspids. These peptides can be put together in numerous and any and all combinations. As an illustrative example only, where a HIV antigen is divided into 5 portions, the portions can be divided equally (i.e. each overlapping fragment is about 21 to 25% of the entire full length if the HIV antigen) or unequally (i.e. a HIV antigen can be divided into the following 5 overlapping fragments; fragment 1 is about 25%, fragment 2 is about 5%, fragment 3 is about 35%, fragment 4 is about 10% and fragment 5 is about 25% of the size of the full length HIV antigen, provided each fragment overlaps with at least one other fragment).

A HIV antigen which is a peptide, (i.e. a lengths of anywhere between 6 residues to 20 residues) can be delivered by a non-linked LF polypeptide. Polypeptides can also by synthesized as branched structures such as those disclosed in U.S. Pat. Nos. 5,229,490 and 5,390,111 which are incorporated herein by reference. Antigenic polypeptides include, for example, synthetic or recombinant B-cell and T-cell epitopes, universal T-cell epitopes, and mixed T-cell epitopes from one organism or disease and B-cell epitopes from another.

As noted above, a HIV antigen can be obtained through recombinant means or peptide synthesis. Other sources include natural sources or extracts. In any event, the antigen can be purified by means of the antigen's physical and chemical characteristics, preferably by fractionation or chromatography (Janson and Ryden, 1989; Deutscher, 1990; Scopes, 1993).

In some embodiments, the vaccine composition as disclosed herein comprise a multivalent HIV antigens, e.g., where more than one HIV antigen is associated with the LFn polypeptide, to induce an immune response to more than one HIV antigen at the same time, e.g., any combination of HIV env, gag, Pol and nef peptides, in any and all combinations. Conjugates can be used to induce an immune response to multiple HIV antigens, to boost the immune response, or both.

### LFn

One aspect of the present invention relates to a therapeutic composition to augment (e.g., increase efficiency) of conventional HIV anti-retroviral therapies for the treatment of subjects with HIV. The development of a vaccine is considered to be very useful for controlling the acquired immune deficiency syndrome (AIDS) epidemic. Such a composition should elicit cytotoxic T lymphocytes, CTL. This can be achieved by immunization with immunogenic peptides or proteins from the infectious agent. However, in the case of human immunodeficiency virus (HIV), these approaches have not yet been successful. Protection against both intravenous and vaginal simian-human immunodeficiency virus (SHIV) challenges by neutralizing antibodies has been shown in macaques (Parren, 2001; Mascola, 2000; Shibata, 1999).

Herein, the inventors have demonstrated that co-administration of a HIV antigen and LFn induces a CTL response to the HIV peptide. In some embodiments, the HIV peptide and LFn are a fusion protein, and in some embodiments, the HIV peptide and LFn are complexed (non-covalently associated) with each other.

B. anthracis is the causative agent of anthrax in animals and humans. The toxin produced by B. anthracis consists of two bipartite protein exotoxins, lethal toxin (LT) and edema toxin. LT is composed of protective antigen (PA) and lethal factor (LF), whereas edema toxin consists of PA and edema factor (EF). The amino-terminal domain from B. anthracis LF is known as LFn. It is the N-terminal 255 amino acids of LF. LF has been found to contain the information necessary for binding to protective antigen (PA) and mediating translocation. The domain alone lacks lethal potential, which depends on the putatively enzymatic carboxyl-terminal moiety (Arora and Leppla 1993, J. Biol. Chem., 268:3334-3341).

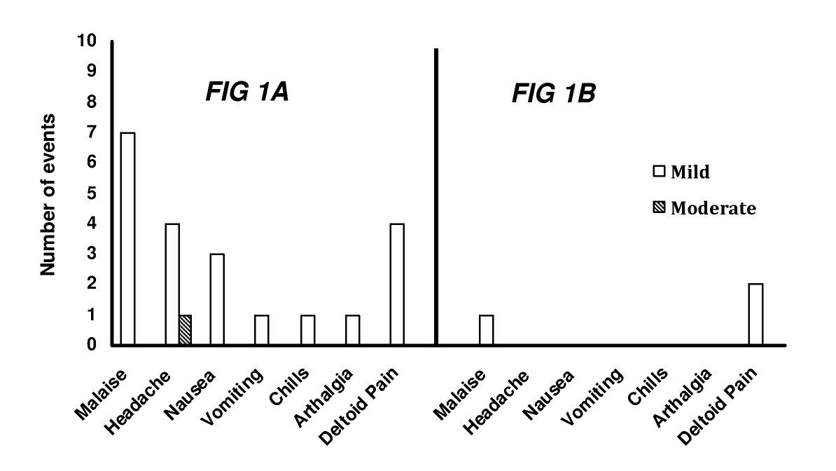

Anthrax lethal factor or LF is a protein, encoded by GenBank Accession Number M29081 (Gene ID No: 143143), that is naturally produced by B. anthracis and that has MAPKK protease activity. The gene encoded B. anthracis LF is a 809 amino acid polypeptide while the mature B. anthracis LF is a 796 amino acid polypeptide after cleavage of the N-terminal leader peptide. Deletion analysis of LF shows that the PA binding domain is located within the amino-terminus of LFn. Mutational studies demonstrate the PA binding domain is located within the region of amino acids 34 to 288 of the LF polypeptide of SEQ ID NO: 1, and within the region of amino acids 1 to 254 of the LF polypeptide of SEQ ID NO: 2 (Arora et al., J. Biol. Chem. 268:3334 3341 (1993); Milne, et al., (1995) Mol. Microbiol. 15, 661-66). The three-dimensional atomic resolution structures of LF have now been solved by X-ray crystallography. Pannifer et al., describe the crystal structure of LF and its complex with a 16-amino acid residue (16-mer) peptide representing the N-terminus of its natural substrate, MAPKK-2, in Nature vol. 414, pg. 229-233 (2001) as a protein that comprises four structural domains: domain I binds the membrane-translocating component of anthrax toxin, the protective antigen (PA); domains II, III and IV together create a long deep groove that holds the 16-residue N-terminal tail of MAPKK-2 before cleavage. Domain I is perched on top of the other three domains, which are intimately connected and comprise a single folding unit. The only contacts between domain I and the rest of the molecule are with domain II, and these chiefly involve charged polar and water-mediated interactions. The nature of the interface is consistent with the ability of a recombinant N-terminal fragment (residues 1-254, excluding the signal peptide) to be expressed as a soluble folded domain that maintains the ability to bind PA and enables the translocation of heterologous fusion proteins into the cytosol (Ballard, J. D., et. al., 1996, Proc. Natl. Acad. Sci. USA 93, 12531-12534; Goletz, T. J. et al., 1997, Proc. Natl. Acad. Sci. USA 94, 12059-12064). Moreover, deletion of the first 36 residues of LFn had no effect on its binding to PA or LF ability to be translocated across membranes (D. Borden Lacy, et. al., 2002, J. Biol. Chem., 277:3006-3010). Domain I consists of a 12-helix bundle that packs against one face of a mixed four-stranded β-sheet, with a large (30-residue) ordered loop, L1, between the second and third-strands forming a flap over the distal face of the sheet (see FIG. 1). The exact docking site on domain I for PA is unknown, but the integrity of the folded domain seems to be required, because a series of insertion and point mutants of buried residues in domain I that presumably disrupt the fold abrogate binding of PA and toxicity (Quinn, C. P., et. al., 1991, J. Biol. Chem., 266: 20124-20130; Gupta, P., et. al., 2001, Biochem. Biophys. Res. Comm., 280:158-163). In addition, LFn has been shown to deliver exogenous protein antigens to the major histocompatibility complex class I pathway in the cytosol of B-cells, CTL-cells and macrophages in the absence of PA (Huyen Cao, et. al., 2002, The Journal of Infectious Diseases; 185:244-251; N. Kushner, et. al., 2003, Proc Natl Acad Sci U S A. 100: 6652-6657). The PA-independent LFn delivery of LFn-fusion proteins depends on functional transport-associated proteins for intracellular antigen processing and transport into the endoplasmic reticulum for binding to MHC class I molecules.

An abrupt turn at the end of the last helix of domain I leads directly into the first helix of domain II (residues 263-297 and 385-550). Although sequence-based comparisons failed to yield any homology, the structural similarity with the catalytic domain of the B. cereus toxin, VIP2 (Protein Data Bank accession code 1QS2), is outstanding. Domain II and VIP2 superimpose with an RMSD of 3.3 Å and a sequence identity of 15%, as determined by DALI (Holm, L. & Sander, 1997, Nucleic Acids Res. 25, 231-234). VIP2 contains an NAD-binding pocket and conserved residues involved in NAD binding and catalysis. Domain II lacks these conserved residues; moreover, a critical glutamic acid that is conserved throughout the family of ADP ribosylating toxins (Carroll, S. F. & Collier, R. J., 1984, Proc. Natl. Acad. Sci. USA 81, 3307-3311) is replaced by a lysine (K518). It is therefore expected that domain II does not have ADP-ribosylating activity.

Domain III is a small α-helical bundle with a hydrophobic core (residues 303-382), inserted at a turn between the second and third helices of domain II. Sequence analysis has revealed the presence of a 101-residue segment comprising five tandem repeats (residues 282-382), and suggested that repeats 2-5 arose from a duplication of repeat 1. The crystal structure reveals that repeat 1 actually forms the second helix-turn element of domain II, whereas repeats 2-5 form the four helix-turn elements of the helical bundle, suggesting a mechanism of creating a new protein domain by the repeated replication of a short segment of the parent domain. Domain III is required for LF activity, because insertion mutagenesis and point mutations of buried residues in this domain abrogate function (Quinn, C. P., et. al., 1991, J. Biol. Chem. 266, 20124-20130). It makes limited contact with domain II, but shares a hydrophobic surface with domain IV. Its location is such that it severely restricts access to the active site by potential substrates such as the loops of a globular protein; that is, it contributes towards specificity for a flexible ‘tail’ of a protein substrate. It also contributes sequence specificity by making specific interactions with the substrate (see below).

Domain IV (residues 552-776) consists of a nine-helix bundle packed against a four-stranded-sheet. Sequence comparisons had failed to detect any homology with other proteins of known structure beyond the HExxH motif. The three-dimensional structure reveals that the β-sheet and the first six helices can be superimposed with those of the metalloprotease thermolysin, with an RMSD of 4.9 Å over 131 residues. Large insertions and deletions occur elsewhere within the loops connecting these elements, so that the overall shapes of the domains are quite different. In particular, a large ordered loop (L2) inserted between strands 42 and 43 of the sheet partly obscures the active site, packs against domain II, and provides a buttress for domain III.

A zinc ion (Zn2+) is coordinated tetrahedrally by a water molecule and three protein side chains, in an arrangement typical of the thermolysin family. Two coordinating residues are the histidines from the HExxH motif (H is 686 and H is 690) lying on one helix (44), as expected. The structure reveals that the third coordinating residue is Glu 735 from helix 46. Glu 687 from the HExxH motif lies 3.5 Å from the water molecule, well positioned to act as a general base to activate the zinc-bound water during catalysis. The hydroxyl group of a tyrosine residue (Tyr 728) forms a strong hydrogen bond (O—O distance 2.6 Å) to the water molecule, on the opposite side of Glu 687, and probably functions as a general acid to protonate the amine leaving group.

The gene encoded 809 amino acid polypeptide B. anthracis LF has seven potential N-glycosylation sites located at asparagine positions 62, 212, 286, 478, 712 736, and 757. Within the LFn (1-288), there are three potential N-glycosylation sites, at asparagine positions 62, 212, and 286, all of which have potential of >0.51 according to the NetNGlyc 1.0 Prediction software from the Technical University of Denmark. The NetNglyc server predicts N-Glycosylation sites in proteins using artificial neural networks that examine the sequence context of Asn-Xaa-Ser/Thr sequons.

The gene encoded 809-aa polypeptide B. anthracis LF is not predicted to have any O-glycosylation sites according to the NetOGlyc 3.1 Prediction software from the Technical University of Denmark. The NetOglyc server produces neural network predictions of mucin type GalNAc O-glycosylation sites in proteins.

“LFn polypeptides” include LF polypeptide fragments represented by SEQ ID Nos. 3 and 4, as well as recombinant LFn, and functional LFn, fragments and variants that retain the function to deliver an LFn-fused HIV antigen polypeptide to the cytosol of an intact cell, preferably a living cell. The term “LFn polypeptide” therefore includes functional LFn homologues such as polymorphic variants, alleles, mutants, and closely related interspecies variants that have at least about 60% amino acid sequence identity to LFn and have the function to deliver a fused polypeptide HIV antigen to the cytosol of a cell, as determined using the assays described herein. In particular embodiments, the LFn polypeptides are substantially identical to LFn of SEQ ID NO: 3 and SEQ ID NO: 4 as disclosed herein. In other embodiments, the LFn polypeptides are conservative substitution mutants of LFn of SEQ ID NO: 3 and SEQ ID NO: 4 as disclosed herein. These conservative substitution mutants of LFn can also function to deliver a fused polypeptide HIV antigen to the cytosol of a cell, as determined using the assays described herein. In some embodiments, some functional polymorphic variants, alleles, mutants, and closely related interspecies variants of LFn that function to deliver a HIV antigen polypeptide to an intact cell can be determined by the methods and assays as disclosed in U.S. patent application Ser. No. 10/473,190 which is incorporated herein by reference.

In some embodiments, the vaccine composition useful in the methods and therapeutic compositions as disclosed herein comprises a fragment of LFn which is about 250 amino acids or less, or about 150 amino acids or less, or about 104 amino acids or less, is able to deliver the fused HIV antigen to a cell and is useful in the methods and compositions described herein.

In one embodiment, the therapeutic composition comprises an LFn polypeptide which comprises a non-functional binding site for PA, and thus is a mutant of LFn which does not result in functional binding with PA. Such mutants include, but are not limited to mutants altered at one or more of the residues critical for interacting with PA, such as a mutation in one or more of the following residues: Y22; L188; D187; Y226; L235; H229 (see Lacy et al., J. Biol. Chem., 2002; 277; 3006-3010); D106A; Y108K; E135K; D136K; N140A and K143A (see Melnyk et al., J. Biol. Chem., 2006; 281; 1630-1635 and Cunningham et al., PNAS, 2002; 99; 70497052, which are incorporated herein in their entirety by reference).

In one embodiment, a therapeutic composition as described herein comprises an LFn polypeptide or a fragment thereof. In some embodiments, a therapeutic composition as described herein comprises a fragment at least residues 34-288 of the LFn polypeptide or a fragment thereof. The LFn polypeptide can be an N-terminal (LFn) polypeptide, or conservative substitution variant thereof, that promotes transmembrane delivery to the cytosol of an intact cell. The amino-terminal domain from B. anthracis LF polypeptide is known as LFn. LF binds to protective antigen (PA) and mediates translocation across the cell membrane. The LFn alone lacks lethal potential, which depends on the putatively enzymatic carboxyl-terminal moiety (Arora and Leppla, 1993, J. Biol. Chem., 268:3334-3341). While not wishing to be bound by theory, the LF polypeptide, individually or fused, is thought to function to mediate membrane translocation. It has been shown that a fusion protein of the LFn domain with a foreign antigen can induce CD8 T cell immune responses even in the absence of PA (Kushner, et. al. 2003, PNAS, 100:6652-6657). The LFn polypeptide is a polypeptide comprising the amino acid residues 1-288 of the LF polypeptide and is capable of traversing cell membranes in the absence of the B. anthracis protective antigen (PA). Amino acids 1-288 includes the N-terminal leader sequence. In addition, when a second protein is attached to an LFn or LF polypeptide, this second protein is also transported across membranes into the cytosol along with the LFn or LF polypeptide. Thus, LFn can be used without PA as a carrier to deliver antigens into the cytosol. The LFn or LF polypeptide therefore facilitates and promotes the transmembrane delivery of other proteins.

In one embodiment, the therapeutic composition as described herein can comprise glycosylated proteins. In other words, the LFn, and/or HIV proteins can each be glycosylated proteins. In one embodiment of the therapeutic compositions described herein, individual or fusion polypeptides are O-linked glycosylated. In another embodiment of the compositions described herein, individual or fusion polypeptides are N-linked glycosylated. In yet another embodiment of the compositions described herein, individual or fusion polypeptides are both O-linked and N-linked glycosylated. In other embodiments, other types of glycosylations are possible, e.g. C-mannosylation. In one embodiment of the compositions described herein, the LFn polypeptide is N-glycosylated. Glycosylation of proteins occurs predominantly in eukaryotic cells. N-glycosylation is important for the folding of some eukaryotic proteins, providing a co-translational and post-translational modification mechanism that modulates the structure and function of membrane and secreted proteins. Glycosylation is the enzymatic process that links saccharides to produce glycans, and attaches them to proteins and lipids. In N-glycosylation, glycans are attached to the amide nitrogen of asparagine side chain during protein translation. The three major saccharides forming glycans are glucose, mannose, and N-acetylglucosamine molecules. The N-glycosylation consensus is Asn-Xaa-Ser/Thr, where Xaa can be any of the known amino acids. O-linked glycosylation occurs at a later stage during protein processing, probably in the Golgi apparatus. In O-linked glycosylation, N-acetyl-galactosamine, O-fucose, O-glucose, and/or N-acetylglucosamine is added to serine or threonine residues. One skilled in the art can use bioinformatics software such as NetNGlyc 1.0 and NetOGlyc Prediction softwares from the Technical University of Denmark to find the N- and O-glycosylation sites in a polypeptide in the present invention. The NetNglyc server predicts N-Glycosylation sites in proteins using artificial neural networks that examine the sequence context of Asn-Xaa-Ser/Thr sequons. The NetNGlyc 1.0 and NetOGlyc 3.1 Prediction software can be accessed at the EXPASY website. In one embodiment, N-glycosylation occurs in the HIV antigen polypeptide of the fusion polypeptide described herein.

In another embodiment, N-glycosylation occurs in the LFn polypeptide of a fusion polypeptide described herein, for example, at asparagine positions 62, 212, and/or 286, all of which have the potential of >0.51 according to the NetNGlyc 1.0 Prediction software. Various combinations of N-glycosylation in the fusion polypeptide of the present invention are possible. In some embodiments, the individual and fusion polypeptides described herein have a single N-glycosylation at one of these three sites: asparagine positions 62, 212, and 286 of LFn. In other embodiments, the individual and fusion polypeptides described herein are N-glycosylated at two of these three sites: asparagine positions 62, 212, and 286 of LFn. In another embodiment, the individual and fusion polypeptides described herein is N-glycosylated at all three sites: asparagine positions 62, 212, and 286 of LFn. In yet another embodiment, N-glycosylation occurs in both the HIV antigen polypeptide (HIV p24 antigen) and the LFn polypeptide. In some embodiments, the glycans of the individual and fusion polypeptide described herein are modified, for example, sialyated or asialyated. Glycosylation analysis of proteins is known in the art, for example, via glycan hydrolysis (using enzymes such as N-glycosidase F, EndoS endoglycosidase, sialidase or with 4N trifluoroacetic acid), derivitization, and chromatographic separation such as LC-MS or LC-MS/MS (Pei Chen et. al., 2008, J. Cancer Res. Clin. Oncology, 134: 851-860; Kainz, E. et. al., 2008, Appl. Environ. Microbiol., 74: 1076-1086). LFn is predicted to have no O-linked glycosylation sites of >0.50 potential.

In one embodiment, the intact cell is a living cell with an unbroken, uncompromised plasma membrane. A living cell would generally have a defined differential membrane potential across the membrane, with the inside of the cell being negative with respect to the outside of the cell. In one embodiment, the intact cell is a mammalian cell, including, for example, an antigen-presenting cell.

While the whole of the N-terminal amino acid residues 1-288 (i.e. domain I of crystal structure, Pannifer et. al., 2001, Nature 414:229-233) of the LF polypeptide promotes the transmembrane delivery of other proteins, it should be understood that smaller fragments of domain I can be used in the compositions as disclosed herein and are sufficient to translocated a HIV antigen across cell membrane and promote the transmembrane delivery of HIV proteins, e.g., when fused together as a fusion polypeptide. The x-ray crystal structure of domain I of LF shows 12 alpha helices and four beta sheet secondary protein structures (Pannifer et. al., 2001, supra). Smaller fragments of domain I that preserve these alpha helices and/or beta sheet secondary protein structures of domain I can translocate across cell membrane and promote the transmembrane delivery of other proteins when fused together as a fusion polypeptide. One skilled in the art can determine the presence of alpha helices and beta sheet secondary protein structure in the LFn polypeptide of the fusion polypeptide using methods known in the art, such as circular dichroism (CD).

In one embodiment, the LFn polypeptide of a composition as described herein comprises at least the 60 carboxy-terminal amino acids of SEQ. ID. No. 3, or a conservative substitution variant thereof. In one embodiment, the LFn polypeptide of a composition as described herein consists essentially of 60 carboxy-terminal amino acids of SEQ. ID. No. 3, or a conservative substitution variant thereof. In one embodiment, the LFn polypeptide of a composition as described herein consists of 60 carboxy-terminal amino acids of SEQ. ID. No. 3, or a conservative substitution variant thereof.

In one embodiment, the LFn polypeptide of a composition as described herein comprises at least the 80 carboxy-terminal amino acids of SEQ. ID. No. 3, or a conservative substitution variant thereof. In one embodiment, the LFn polypeptide of a composition as described herein consists essentially of 80 carboxy-terminal amino acids of SEQ. ID. No. 3, or a conservative substitution variant thereof. In one embodiment, the LFn polypeptide of a composition as described herein consists of 80 carboxy-terminal amino acids of SEQ. ID. No. 3, or a conservative substitution variant thereof.

In one embodiment, the LFn polypeptide of a vaccine composition as described herein comprises at least the 104 carboxy-terminal amino acids of SEQ. ID. No. 3, or a conservative substitution variant thereof. In one embodiment, the LFn polypeptide of a vaccine composition as described herein consists essentially of 104 carboxy-terminal amino acids of SEQ. ID. No. 3, or a conservative substitution variant thereof. In one embodiment, the LFn polypeptide of a vaccine composition as described herein consists of 104 carboxy-terminal amino acids of SEQ. ID. No. 3, or a conservative substitution variant thereof.

In one embodiment, the LFn polypeptide of a composition as described herein comprises the amino acid sequence corresponding to SEQ. ID. No. 5, or a conservative substitution variant thereof. In one embodiment, the LFn polypeptide of a composition as described herein consists essentially of the amino acid sequence corresponding to SEQ. ID. No. 5, or a conservative substitution variant thereof. In one embodiment, the LFn polypeptide of a composition as described herein consists of the amino acid sequence corresponding to SEQ. ID. No. 5, or a conservative substitution variant thereof.

In one embodiment, the LFn polypeptide of a composition as described herein comprises the amino acid sequence corresponding to SEQ. ID. No. 4, or a conservative substitution variant thereof. In another embodiment, the LFn polypeptide of a composition as described herein consists essentially of the amino acid sequence corresponding to SEQ. ID. No. 4, or a conservative substitution variant thereof. In yet another embodiment, the LFn polypeptide of a composition as described herein consists of the amino acid sequence corresponding to SEQ. ID. No. 4, or a conservative substitution variant thereof.

In one embodiment, the LFn polypeptide of a composition as described herein comprises the amino acid sequence corresponding to SEQ. ID. No. 3, or a conservative substitution variant thereof. In another embodiment, the LFn polypeptide of a composition as described herein consists essentially of the amino acid sequence corresponding to SEQ. ID. No. 3, or a conservative substitution variant thereof. In yet another embodiment, the LFn polypeptide of a composition as described herein consists of the amino acid sequence corresponding to SEQ. ID. No. 3, or a conservative substitution variant thereof.

In one preferred embodiment, the LFn polypeptide of a composition as described herein, promotes transmembrane delivery of the HIV antigen.

In one embodiment, the LFn polypeptide of a composition as described herein, the LFn polypeptide does not bind B. anthracis protective antigen (PA) protein. The PA protein is the natural binding partner of LF, forming bipartite protein exotoxin, lethal toxin (LT). The PA protein is a 735-amino acid polypeptide, a multi-functional protein that binds to cell surface receptors, mediates the assembly and internalization of the complexes, and delivers them to the host cell endosome. Once PA is attached to the host receptor, it is cleaved by a host cell surface (furin family) protease before it is able to bind LF. The cleavage of the N-terminus of PA enables the C-terminal fragment to self-associate into a ring-shaped heptameric complex (prepore) that can bind LF and delivers LF into the cytosol. The N-terminal fragment (residues 1-288, domain I) can be expressed as a soluble folded domain that maintains the ability to bind PA and enables the translocation of heterologous fusion proteins into the cytosol. Smaller fragments of this residues 1-288 N-terminal fragment have been shown to also translocate heterologous fusion proteins into the cytosol in the absence of PA. Hence, in one embodiment, smaller fragments described herein can translocate across membranes but do not bind PA.

In one embodiment, the LFn polypeptide of a composition as described herein, the LFn polypeptide substantially lacks amino acids 1-33 of SEQ. ID. No. 3. Amino acids 1-33 of SEQ. ID. No. 3 encompass the signal peptide that is predicted to direct the post-translational transport of the LF protein. In some embodiments, the LFn polypeptide of any of the fusion polypeptides described herein lacks a signal peptide that functions to direct the post-translational transport of the fusion polypeptide. In other embodiments, the LFn polypeptide of the fusion polypeptides described herein comprises a signal peptide for co-translation on the ER. The signal peptide is also called a leader peptide in the N-terminus, which may or may not be cleaved off after the translocation through the ER membrane. One example of a signal peptide is MAPFEPLASGILLLLWLIAPSRA (SEQ. ID. No. 17). Other examples of signal peptides can be found at SPdb, a Signal Peptide Database, which is found at the world wide web site of http colon “forward slash” “forward slash” proline “period” bic “period” nus “period” edu “period” sg “forward slash” spdb “forward slash”.

In some embodiments, an LFn mimetic is useful in the compositions and methods described herein. An “LFn mimetic” refers to a compound or molecule, e.g., a peptide, polypeptide, or small chemical molecule that functions as LFn to deliver a target antigen to the cytosol of a cell to induce a CMI response against the antigen. LFn mimetics thus include LFn homologues. LFn mimetics would also include small LFn peptides that retain the LFn function to deliver polypeptide antigens (not linked to the LFn mimetic) to the cytosol of the cell, and conservatively substituted variants thereof, as well as truncated versions of LFn that retain ability of LFn to deliver polypeptide antigens (not linked to the LFn mimetic) to the cytosol of a cell. LFn mimetics are tested using assays for a CMI response to the target antigen as disclosed herein and in the Examples of U.S. patent application Ser. No. 10/473,190 (which is incorporated herein in its entirety by reference), e.g., induction of a CTL response to the delivered target antigen. When testing for an LFn mimetic, LFn is typically used as a positive control for delivery of the target antigen to a cell.

### Conventional Antiretroviral Therapies

In some embodiments, the therapeutic composition as disclosed herein can be administered during the continuous administration of a conventional anti-retroviral therapy. In some embodiments, the composition as disclosed herein can be administered immediately after the stopping of a continuous administration of a conventional anti-retroviral therapy.

In some embodiments, the composition as disclosed herein can be administered at a precise point during the continuous administration of one type conventional anti-retroviral therapy, and after a pre-determined period of time after administration of the composition, the continuous administration of the conventional anti-retroviral therapy can be stopped for a period of time. In some embodiments, the conventional anti-retroviral therapy can be stopped for 1 day, or 1 week, or longer than 1 week, e.g., at least 2 weeks, or at least about 3 week or at least about 4 weeks, or more than 4 weeks. In some embodiments, when the conventional anti-retroviral therapy is restarted, the subject can be administered the same or a different type of continuous anti-retroviral therapy.

Anti-retroviral therapies are well known in the art and are encompassed for use in the methods as disclosed herein. For example, various anti-viral compounds known in the art may also be included in the combination therapy according to the present invention. Conventional anti-retroviral compounds suitable for use in combination with the composition as disclosed herein include cells (e.g., stem cell therapy), nucleic acids, polypeptides and other active agents, including, but are not limited to, HIV protease inhibitors, nucleoside HIV reverse transcriptase inhibitors, non-nucleoside HIV reverse transcriptase inhibitors, and HIV integrase inhibitors.

Examples of nucleoside HIV reverse transcriptase inhibitors include 3′-Azido-3′-deoxythymidine (Zidovudine, also known as AZT and RETROVIR®), 2′,3′-Didehydro-3′-deoxythymidine (Stavudine, also known as 2′,3′-dihydro-3′-deoxythymidine, d4T, and ZERIT®), (2R-cis)-4-Amino-1-[2-(hydroxymethyl)-1,3-oxathiolan-5-yl]-2(1H)-pyrimidi-none (Lamivudine, also known as 3TC, and EPIVIR®), and 2′,3′-dideoxyinosine (ddI).

Examples of non-nucleoside HIV reverse transcriptase inhibitors include (−)-6-Chloro-4-cyclopropylethynyl-4-trifluoromethyl-1,4-dihydro-2-H-3,1-benzoxazin-2-one (efavirenz, also known as DMP-266 or SUSTIVA®) (see U.S. Pat. No. 5,519,021), 1-[3-[(1-methylethyl)aminol]-2-pyridinyl]-4-[[5-[(methylsulfonyl)amino]-1-H-indol-2-yl]carbonyl]piperazine (Delavirdine, see PCT International Patent Application No. WO 91/09849), and (1S,4R)-cis-4-[2-amino-6-(cycloprpoylamino)-9H-purin-9-yl]-2-cyclopentene-1-1-methanol (Abacavir).

Examples of protease inhibitors include [5S-(5R*,8R*,10R*,11R*)]-10-hydroxy-2-methyl-5-(1-methylethyl)-1-[2-(1-me-thylethyl)-4-thiazolyl]-3,6-dioxo-8,11-bis(phenylmethyl)-2,4,7,12-tetraaza-tridecan-13-oic acid 5-thiazolylmethyl ester (Ritonavir, marketed by Abbott as NORVIR®), [3S-[2(2S*,3S*),3a,4ab,8ab]]-N-(1,1-dimethylethyl)decahydro-2-[2-hydroxy-3-[(3-hydroxy-2-methylbenzoyl)amino]-4-(phenylthio)butyl]-3-isoquinolineca-rboxamide monomethanesulfonate (Nelfinavir, marketed by Agouron as VIRACEPT®), N-(2(R)-hydroxy-1(S)-indanyl)-2(R)-phenylmethyl-4-(S)-hydroxy-5-(1-(4-(2-benzo[b]furanylmethyl)-2(S)—N(t-butylcarboxamido)-piperazinyl))-pentaneam-ide (See U.S. Pat. No. 5,646,148), N-(2(R)-hydroxy-1(S)-indanyl)₂(R)-phenylmethyl-4-(S)-hydroxy-5-(1-(4-(3-p-yridylmethyl)-2(S)—N′-(t-butylcarboxamido)-piperazinyl))-pentaneamide (Indinavir, marketed by Merck as CRIXIVAN®), 4-amino-N-((2 syn,3S)-2-hydroxy-4-phenyl-3-((S)-tetrahydrofuran-3-yloxycarbonylamino)-b-utyl)-N-isobutyl-benzenesulfonamide (amprenavir, see U.S. Pat. No. 5,585,397), and N-tert-butyl-decohydro-2-[2(R)-hydroxy-4-phenyl-3(S)—[[N-(2-quinolylcarb-onyl)-L-asparaginyl]amino]butyl]-(4aS,8aS)-isoquinoline-3(S)-carboxamide (Saquinavir, marketed by Roche Laboratories as INVIRASE®).

Examples of suitable HIV integrase inhibitors are disclosed in U.S. Pat. Nos. 6,110,716; 6,124,327; and 6,245,806, which are incorporated herein by reference.

In addition, antifusogenic peptides disclosed in, e.g., U.S. Pat. No. 6,017,536 can also be included in the combination therapies according to the present invention. Such peptides typically consist of a 16 to 39 amino acid region of a simian immunodeficiency virus (SIV) protein and are identified through computer algorithms capable of recognizing the ALLMOTI5, 107.times.178.times.4, or PLZIP amino acid motifs. See U.S. Pat. No. 6,017,536, which is incorporated herein by reference.

In some embodiments, the conventional anti-retroviral therapy includes combination therapies, which refers to the continuous administration of a combination of two or more anti-retroviral drugs or active agents such as HIV protease inhibitors, nucleoside HIV reverse transcriptase inhibitors, non-nucleoside HIV reverse transcriptase inhibitors, and HIV integrase inhibitors. In such combination therapy, two or more anti-HIV agents can be administered in the same pharmaceutical composition or administered separately. Thus, the present invention also encompasses compositions use of the composition as disclosed herein to allow breaks or intermittent stopping of combination therapies as described above.

For example, conventional anti-retroviral therapies which are combination therapies are well known to persons of ordinary skill in the art. For example, these include, but are not limited to Tenofovir, which is a new nucleotide reverse transcriptase inhibitor recently approved in the United States for the treatment of HIV-1 infection in combination with other antiretroviral agents. Nucleotide analogues are very similar to nucleoside analogues but are pre-phosphorylated, and thus require less processing by the body. Tenofovir DF (disoproxil fumarate) is described in U.S. Pat. Nos. 5,935,946, 5,922,695, 5,977,089, 6,043,230 & 6,069,249 while PMPA or Tenofovir DF is described in U.S. Pat. Nos. 4,808,716, 5,733,788 & 6,057,305, which are incorporated herein in their entirety by reference. Similarity, US2004/0224917 describes the combination of Tenofovir DF and Emtricitabine. Other various antiretroviral combinations have been made available for avoiding the development of HIV resistant strains and to formulate combination regimens. One example is the combination of synthetic nucleoside analogues Lamivudine (150 mg) and Zidovudine (300 mg), which is commercially available as Combivir® of GlaxoSmithKline. Another such combination is of the nucleoside analogues Abacavir and Lamivudine, which is described in Glaxo's patent application no WO 03/101467 which is incorporated herein in its entirety by reference. Lamivudine (also known as 3TC) and its use in the treatment and prophylaxis of viral infections are described in U.S. Pat. No. 5,047,407 which is incorporated herein in its entirety by reference. Lamivudine and its use against HIV are described in WO 91/17159 and EP 0382526 which are incorporated herein in its entirety by reference. Crystalline forms of lamivudine are described in WO 92/21676 which is incorporated herein in its entirety by reference. Combinations of lamivudine with other nucleoside reverse transcriptase inhibitors, in particular zidovudine AZT, are described in WO 92/20344, WO 98/18477, and WO/9955372, which are incorporated herein in their entirety by reference.

Antiretroviral therapies also include various non-nucleoside reverse transcriptase inhibitors (NNRTIs), are known, such as delavirdine, capravirine, Efavirenz and nevirapine. NNRTIs are common components of therapy for antiretroviral-naive HIV-infected patients, and provide synergistic activity with nucleoside reverse transcriptase inhibitors (NRTIs). Efavirenz is chemically known as (S)-6-chloro-4-(cyclopropylethynyl)-1,4-dihydro-4-(trifluoromethyl)-2H-3,-1-benzoxazin-2-one. Efavirenz is a HIV-1 specific, non nucleoside, reverse transcriptase inhibitor. Efavirenz is useful for the treatment of HIV and has been reported to inhibit reproduction of HIV in the body. Efavirenz is commercially available from Bristol-Myers Squibb Co, under the name SUSTIVA®, for treatment of HIV, and is described, for example, in U.S. Pat. Nos. 5,519,021; 5,663,1699; 5,811,423 and 6,238,695, which are incorporated herein in their entirety by reference. Nevirapine, chemically, 11-Cyclopropyl-5,11-dihydro-4-methyl-6H-dipyrido[3,2-b: 2′,3′-e][1,4]diazepin-6-one is a non-nucleoside reverse transcriptase inhibitor. The therapeutic uses of nevirapine and related compounds and their preparations are described in U.S. Pat. No. 5,366,972, which is incorporated herein by reference. Nevirapine is commercially available as 200 mg tablet and 50 mg/5 mL in 240 mL oral suspension. It is sold under the name VIRAMUNE®.

Other antiretroviral drugs are discussed in U.S. application 2008/0317852 which is incorporated herein in its entirety by reference.

Treatment Regimens

As discussed herein, one aspect of the present invention relates to administering a pharmaceutical composition as disclosed herein comprising a HIV antigen and an LFn polypeptide to a subject in combination with traditional anti-retroviral therapy or combination HIV viral therapy to augment, e.g., enhance the traditional HIV anti-retroviral therapy. Accordingly, the present invention relates to a dual therapeutic approach using the present compositions on a periodic basis (e.g., pulsed administration), in combination with traditional combination retroviral therapy to enhance the efficacy of the traditional retroviral therapy in subjects positive for HIV or suffering from AIDS.

In some embodiments, the pharmaceutical composition as disclosed herein can be administered during (e.g., at the same time as) the continuous administration of a conventional anti-retroviral therapy. The “continuous administration of a conventional anti-retroviral therapy” refers to an anti-retroviral therapy which is administered to the subject on a regular and frequent basis without any breaks in the regimen, e.g., more than twice a day, twice a day, daily, every other day, once a week etc. Accordingly, in some embodiments, one can administer the composition once or twice during the normal regimen of the administration of a conventional HIV anti-retroviral therapy.

In some embodiments, the composition as disclosed herein can be administered immediately after the stopping of a continuous administration of a conventional anti-retroviral therapy. In some embodiments for example, one can stop the daily or weekly regimen of conventional HIV anti-retroviral treatment and on same day, or one or more days before the stopping of the daily regimen, the subjects can be vaccinated with the present composition as disclosed herein.

In some embodiments, the composition as disclosed herein can be administered at a pre-determined point in the regimen of continuous administration of a conventional HIV anti-retroviral therapy, and after a pre-determined period of time after administration of the composition, the regimen of continuous administration of the conventional HIV anti-retroviral therapy can be stopped for a period of time. In some embodiments, the conventional anti-retroviral therapy can be decreased in dose or completely stopped for at least 1 day, or 1 week, or longer than 1 week. For example, the conventional anti-retroviral therapy can be decreased in dose for a period of at least 2 weeks, or at least about 3 week or at least about 4 weeks, or more than 4 weeks, e.g., 1 month, 6 weeks, 2 months or more than 2 months. In some embodiments, when the conventional anti-retroviral therapy is restarted, the subject can be administered the same or a different type of continuous anti-retroviral therapy.

In some embodiments, the subject can be treated with the composition as disclosed herein at least 1 day, or at least 2 days, or at least 3 days or at least 4 days, or at least about 5 days, or at least about 1 week, or at least about 10 days, or at least about 2 weeks, or at least about 3 weeks, or at least about 1 month prior, or more than 1 month prior to decreasing the dose and/or stopping a regimen of continuous administration of the conventional HIV anti-retroviral therapy.

In some embodiments, the composition is administered to the subject undergoing conventional HIV anti-retroviral therapy at least once a month, at least every other month, or at least every 6 months, or at least every year, or every other year.

Accordingly, as a subject administered the compositions can decrease the dose of the conventional HIV anti-retroviral therapy for a period of time, the total daily dose of the conventional HIV anti-retroviral therapy can be decreased to more than 25% and less than 75% of the normal dose for the conventional HIV anti-retroviral therapy. In other embodiments, administration with the composition as disclosed herein allows a decrease in the dose of the conventional HIV anti-retroviral therapy to less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2%, or less than 1% of the normal dose of the conventional HIV anti-retroviral therapy (e.g., before the composition was administered to the subject).

In alternative embodiments, as a subject administered the compositions can take breaks or interruptions from the regular dosing regimen of the conventional HIV anti-retroviral therapies, one can use the compositions as disclosed herein to allow pulse administration of the conventional HIV anti-retroviral therapies. For example, in some embodiments, the administration of a conventional HIV anti-retroviral therapy can be administered by pulsed administration. In certain embodiments, a subject administered the compositions can have pulsed administration of the conventional HIV anti-retroviral therapy which comprises administering the conventional HIV anti-retroviral therapy for a first time period, followed by not administering the conventional HIV anti-retroviral therapy for a second time period. In some embodiments, the first time period is about at least 1 week, or at least about 1 month, or at least about 2 months, or at least about 3 months or more than 3 months. In some embodiments, the second time period, which typically occurs after a predetermined time after administration of the composition as disclosed herein, can be for at least 1 day, or 1 week, or longer than 1 week, or at least 2 weeks, or at least about 3 week or at least about 4 weeks, or more than 4 weeks, e.g., about 1 month, or about 6 weeks, or about 2 months or more than 2 months.

In some embodiments, the duration of first time period (e.g., regular regimen of conventional HIV anti-retroviral therapy is administered) is the same length as the duration of second time period (e.g. the duration of the break or “drug holiday” where the regimen of conventional HIV anti-retroviral therapy is halted). As a non-limiting example, the duration of the first time period can be 1 month, followed by the duration of the second time period of 1 month. In some embodiments, the duration of the first time period (e.g., regular regimen of conventional HIV anti-retroviral therapy is administered) is longer than the duration of the second time period (e.g. the duration of the break or “drug holiday”). As a non-limiting example, the duration of the first time period can be 2 months, followed by the duration of the second time period of less than 2 months, e.g., at least 1 day, or 1 week, or about 2 weeks, or about 3 weeks or about 4 weeks, or more than 4 weeks but less than 2 months.

In certain embodiments, pulsed administration of the conventional HIV anti-retroviral therapy can repeat provided the subject has been administered the composition as disclosed herein at sufficient regular intervals to keep the HIV viral load low during the second time periods (e.g., during the entire duration of the drug break or “drug holiday” where the conventional HIV anti-retroviral therapy is not administered). In certain embodiments, the composition allows a subject to undergo pulsed administration of the conventional HIV anti-retroviral therapy for the lifetime of the subject.

In some embodiments, the composition is administered to the subject that is undergoing a pulsed administration of the conventional HIV anti-retroviral therapy least once a month, at least every other month, or at least every 6 months, or at least every year, or every other year.

Compositions and Formulations and Administration

In one embodiment, provided herein is a method of augmenting (e.g., increasing the effectiveness) of a conventional HIV anti-retroviral treatment in a subject with HIV by administrating the subject with a composition comprising a HIV antigen and a LFn polypeptide or a fragment thereof.

In another embodiment, provided herein is a method of immunizing a mammal against an HIV, the method comprising administering a composition comprising, as a HIV antigen, a preparation comprising, or alternatively, of a HIV polypeptide.

In another embodiment, provided herein is a method of immunizing a mammal against a HIV, the method comprising administering a composition comprising a pharmaceutically acceptable carrier, a B. anthracis Lethal Factor (LF) polypeptide, e.g., LFn or residues 34-288 of LFn (e.g. without the signal peptide) and an antigen preparation, the antigen preparation comprising a fragment of the HIV polypeptide, e.g., p24.

In one embodiment, the compositions described herein comprise a polypeptide that is expressed and purified from insect cells. In one embodiment, the composition comprises a plurality of HIV polypeptide, or fragments thereof, that are expressed and purified from insect cells. In another embodiment, the composition comprises an LF polypeptide, e.g., LFn, wherein the LF polypeptide is N-glycosylated. The N-glycosylation can be at asparagine 62, 212 and/or 286.

In one embodiment, the compositions described herein comprise a pharmaceutically acceptable carrier. In another embodiment, the composition as described herein is formulated for administering to a mammal. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

In one embodiment, a composition as described herein comprise pharmaceutically acceptable carriers that are inherently nontoxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. Nos. 3,773,919, 3,887,699, EP 58,481A, EP 158,277A, Canadian Patent No. 1176565; U. Sidman et al., Biopolymers 22:547 (1983) and R. Langer et al., Chem. Tech. 12:98 (1982). The proteins will usually be formulated at a concentration of about 0.1 mg/ml to 100 mg/ml per application per patient.

In one embodiment, other ingredients can be added to vaccine formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In one embodiment, the compositions as described herein for administration must be sterile. Sterility is readily accomplished by filtration through sterile filtration membranes (e.g., 0.2 micron membranes) or other known technique commonly known in the art.

In some embodiments, the composition as described herein further comprises pharmaceutical excipients including, but not limited to biocompatible oils, physiological saline solutions, preservatives, carbohydrate, protein, amino acids, osmotic pressure controlling agents, carrier gases, pH-controlling agents, organic solvents, hydrophobic agents, enzyme inhibitors, water absorbing polymers, surfactants, absorption promoters and anti-oxidative agents. Representative examples of carbohydrates include soluble sugars such as hydropropyl cellulose, carboxymethyl cellulose, sodium carboxyl cellulose, hyaluronic acid, chitosan, alginate, glucose, xylose, galactose, fructose, maltose, saccharose, dextran, chondroitin sulfate, etc. Representative examples of proteins include albumin, gelatin, etc. Representative examples of amino acids include glycine, alanine, glutamic acid, arginine, lysine, and their salts.

In some embodiments, the polypeptides described herein can be solubilized in water, a solvent such as methanol, or a buffer. Suitable buffers include, but are not limited to, phosphate buffered saline Ca²⁺/Mg²⁺ free (PBS), normal saline (150 mM NaCl in water), and Tris buffer. Antigen not soluble in neutral buffer can be solubilized in 10 mM acetic acid and then diluted to the desired volume with a neutral buffer such as PBS. In the case of antigen soluble only at acid pH, acetate-PBS at acid pH may be used as a diluent after solubilization in dilute acetic acid. Glycerol can be a suitable non-aqueous buffer for use in the present invention.

If the polypeptide is not soluble per se, the polypeptide can be present in the formulation in a suspension or even as an aggregate. In some embodiments, hydrophobic antigen can be solubilized in a detergent, for example a polypeptide containing a membrane-spanning domain. Furthermore, for formulations containing liposomes, an antigen in a detergent solution (e.g., a cell membrane extract) may be mixed with lipids, and liposomes then may be formed by removal of the detergent by dilution, dialysis, or column chromatography.

In some embodiments, the composition is administered in combination with other therapeutic ingredients including, e.g., γ-interferon, cytokines, chemotherapeutic agents, or anti-inflammatory or anti-viral agents.

In some embodiments, the composition is administered in a pure or substantially pure form, but it is preferable to present it as a pharmaceutical composition, formulation or preparation. Such formulation comprises polypeptides described herein together with one or more pharmaceutically acceptable carriers and optionally other therapeutic ingredients. Other therapeutic ingredients include compounds that enhance antigen presentation, e.g., gamma interferon, cytokines, chemotherapeutic agents, or anti-inflammatory agents. The formulations can conveniently be presented in unit dosage form and may be prepared by methods well known in the pharmaceutical art. For example, Plotkin and Mortimer (In ‘Vaccines’, 1994, W.B. Saunders Company; 2nd edition) describes vaccination of animals or humans to induce an immune response specific for particular pathogens, as well as methods of preparing antigen, determining a suitable dose of antigen, and assaying for induction of an immune response.

In some embodiments, the composition as described herein further comprises an adjuvant. Adjuvants are a heterogeneous group of substances that enhance the immunological response against an antigen that is administered simultaneously. In some instances, adjuvants are added to a vaccine to improve the immune response so that less vaccine is needed. Adjuvants serve to bring the antigen—the substance that stimulates the specific protective immune response—into contact with the immune system and influence the type of immunity produced, as well as the quality of the immune response (magnitude or duration). Adjuvants can also decrease the toxicity of certain antigens; and provide solubility to some vaccine components. Almost all adjuvants used today for enhancement of the immune response against antigens are particles or form particles together with the antigen. In the book “Vaccine Design—the subunit and adjuvant approach” (Ed: Powell & Newman, Plenum Press, 1995) almost all known adjuvants are described both regarding their immunological activity and regarding their chemical characteristics. The type of adjuvants that do not form particles are a group of substances that act as immunological signal substances and that under normal conditions consist of the substances that are formed by the immune system as a consequence of the immunological activation after administration of particulate adjuvant systems.

In one embodiment, a composition as described herein further comprise an adjuvant. Examples of adjuvants include, but are not limited to QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59.

In some embodiments, suitable adjuvants include, but are not limited to, alum, MF59, LTR72 (a mutant of E. coli heat-labile enterotoxin with partial knockout of ADP-ribosyltransferase activity), polyphosphazine adjuvant, interleukins such as IL-1, IL-2, IL-4, IL-6, IL-8, IL-10 and IL-12, interferons such as alpha-interferon and gamma-interferon, tumor necrosis factor (TNF), platelet derived growth factor (PDGF), GCSF, granulocyte-macrophage colony-stimulating factor (GM-CSF), epidermal growth factor (EGF), and the like. Examples of adjuvants capable of stimulating cellular immune responses include cytokines secreted by helper T cells called Th1 cells, e.g., interleukin-2 (IL-2), interleukin-4, interleukin-12 (IL-12) and interleukin-18, fusion proteins having one of such Th1 type cytokines (e.g., IL-2) fused to the Fc portion of immunoglobulin G (IgG), interferons such as alpha-interferon, beta-interferon and gamma-interferon, and chemokines that attract T cells to infected tissues. Non-coding, ISS-enriched plasmid DNAs or ISS oligonucleotides (ISS-ODNs) can also be used in the present invention as adjuvants to enhance cellular immunity.

Using particulate systems as adjuvants, the antigens are associated or mixed with or into a matrix, which has the characteristics of being slowly biodegradable. Care must be taken to ensure that that the matrices do not form toxic metabolites. Preferably, the main kinds of matrices used are mainly substances originating from a body. These include lactic acid polymers, poly-amino acids (proteins), carbohydrates, lipids and biocompatible polymers with low toxicity. Combinations of these groups of substances originating from a body or combinations of substances originating from a body and biocompatible polymers can also be used. Lipids are the preferred substances since they display structures that make them biodegradable as well as the fact that they are a critical element in all biological membranes.

Adjuvants for vaccines are well known in the art. Examples include, but not limited to, monoglycerides and fatty acids (e.g. a mixture of mono-olein, oleic acid, and soybean oil); mineral salts, e.g., aluminium hydroxide and aluminium or calcium phosphate gels; oil emulsions and surfactant based formulations, e.g., MF59 (microfluidised detergent stabilised oil-in-water emulsion), QS21 (purified saponin), AS02 [SBAS2] (oil-in-water emulsion+MPL+QS-21), Montanide ISA-51 and ISA-720 (stabilised water-in-oil emulsion); particulate adjuvants, e.g., virosomes (unilamellar liposomal vehicles incorporating influenza haemagglutinin), ASO₄ ([SBAS4] Al salt with MPL), ISCOMS (structured complex of saponins and lipids), polylactide co-glycolide (PLG); microbial derivatives (natural and synthetic), e.g., monophosphoryl lipid A (MPL), Detox (MPL+M. Phlei cell wall skeleton), AGP [RC-529] (synthetic acylated monosaccharide), DC_Chol (lipoidal immunostimulators able to self organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects); endogenous human immunomodulators, e.g., hGM-CSF or hIL-12 (cytokines that can be administered either as protein or plasmid encoded), Immudaptin (C3d tandem array) and inert vehicles, such as gold particles. Newer adjuvants are described in U.S. Pat. No. 6,890,540, U.S. Patent Application No. 2005/0244420, and PCT/SE97/01003, the contents of which are incorporated herein by reference in their entirety.

In some embodiments, the compositions described herein can be administered intravenously, intranasally, intramuscularly, subcutaneously, infraperitoneally or orally. In some embodiments, the route of administration is oral, intranasal or intramuscular.

Accordingly, in some embodiments, the composition as disclosed herein is formulated suitable for intravenous, intramuscular, intranasal, oral, subcutaneous, or intraperitoneal administration. Such formulations typically comprise sterile aqueous solutions of the active ingredient with solutions which are preferably isotonic with the blood of the recipient. Such formulations may be conveniently prepared by dissolving solid active ingredient in water containing physiologically compatible substances such as sodium chloride (e.g., 0.1-2.0 M), glycine, and the like, and having a buffered pH compatible with physiological conditions to produce an aqueous solution, and rendering the solution sterile. These may be present in unit or multi-dose containers, for example, sealed ampoules or vials.

Liposomal suspensions can also be used as pharmaceutically acceptable carriers. These can be prepared according to methods known to those skilled in the art, for example, as described in U.S. Pat. No. 4,522,811, which is incorporated herein in its entirety by reference.

Formulations for an intranasal delivery are described in U.S. Pat. Nos. 5,427,782, 5,843,451 and 6,398,774, which are incorporated herein in their entirety by reference.

In some embodiments, the formulations of the compositions can incorporate a stabilizer. Illustrative stabilizers are polyethylene glycol, proteins, saccharide, amino acids, inorganic acids, and organic acids which may be used either on their own or as admixtures. Two or more stabilizers may be used in aqueous solutions at the appropriate concentration and/or pH. The specific osmotic pressure in such aqueous solution is generally in the range of 0.1-3.0 osmoses, preferably in the range of 0.80-1.2. The pH of the aqueous solution is adjusted to be within the range of 5.0-9.0, preferably within the range of 6-8.

In some embodiments, when oral preparations are desired, the compositions can be combined with typical carriers, such as lactose, sucrose, starch, talc magnesium stearate, crystalline cellulose, methyl cellulose, carboxymethyl cellulose, glycerin, sodium alginate or gum arabic among others.

A method of immunization or injecting a mammal for augmenting a HIV conventional anti-retroviral therapy comprises administering a vaccine composition as described herein.

The administration, which can be a vaccination, with the compositions as disclosed herein can be conducted by conventional methods. For example, a polypeptide can be used in a suitable diluent such as saline or water, or complete or incomplete adjuvants. The composition can be administered by any route appropriate for eliciting an immune response. The composition can be administered once or at periodic intervals until an immune response is elicited. Immune responses can be detected by a variety of methods known to those skilled in the art, including but not limited to, antibody production, cytotoxicity assay, proliferation assay and cytokine release assays. For example, samples of blood can be drawn from the immunized mammal, and analyzed for the presence of antibodies against the NP, M1, and/or M2 proteins by ELISA (see de Boer G F, et. al., 1990, Arch Virol. 115:47-61) (e.g. using The ImmTech Influenza A Nucleoprotein Antigen Capture ELISA kits (IAV-1192 and IVA-1480) and the titer of these antibodies can be determined by methods known in the art.

The precise dose to be employed in the formulation will also depend on the route of administration and should be decided according to the judgment of the practitioner and each patient's circumstances. For example, a range of 25 μg-900 μg total protein can be administered intradermally, monthly for about 3 months or more.

Ultimately, the attending physician will decide the amount of protein or composition to administer to particular individuals.

### Methods of Measuring or Detecting Protein-Protein Interaction

Methods of measuring or detecting protein-protein interaction are well known. One skilled in the art can determine PA binding activity, for example, by mixing and incubating PA63 with LFn for a period of time, chemically cross-linking any complex formed and analysis of the covalently linked complex by gel electrophoresis or by radioactivity counting as described by Quinn C P. et. al., 1991, J. Biol. Chem. 266:20124-20130. Briefly, the binding assay is determined at 5° C. by competition with radiolabeled 125 I-LFn. Native LF or full-length N-terminal (amino acid 1-288) LFn is radiolabeled (˜7.3×106 cpm/μg protein) using Bolton-Hunter reagent (Amersham Corp). For binding studies, J774A.1 cells cultured in 24-well tissue culture plates are cooled by incubating at 4° C. for 60 min and then placing the plates on ice. The medium is then replaced with cold (4° C.) minimal essential medium containing Hanks' salts (GIBCO®/BRL) supplemented with 1% (w/v) bovine serum albumin and 25 mM HEPES (binding medium). Native PA (0.1 g/ml) is added with radiolabeled native LF (¹²⁵I-LF, 0.1 μg/ml, 7.3×106 cpm/μg) and the plates incubated for 14 h on wet ice. Mutant LF proteins were assayed at varying concentrations for their ability to compete with native ¹²⁵I-LF. For quantitation of bound, radiolabeled LF, cells were gently washed twice in cold binding medium, once in cold Hanks' balanced salt solution, solubilized in 0.50 ml of 0.1 M NaOH, and counted in a gamma counter (Beckman Gamma 9000).

### Methods of Determining Membrane Translocation

In some embodiments, one can determine if the composition as disclosed herein has elicited an immune response in the subject to the HIV antigen by determining membrane translocation of the HIV antigen. Methods of determining membrane translocation are well known in the art, for example, in Wesche, J., et. al., 1998, Biochemistry 37: 15737-15746 and Sellman, B. R., et. al., 2001, J. Biol. Chem. 276: 8371-8376. For example, CHO-K1 cells in a 24-well plate are chilled on ice, washed, and incubated on ice for 2 h with any of the LFn-HIV antigen fusion polypeptides described herein (or a conservative substitution variant thereof or fragments of domain I) that have been labeled with [³⁵S]methionine in an in vitro transcription/translation system (Promega). The cells then are washed with ice-cold PBS at pH 5.0 or 8.0, incubated at 37° C. for 1 min, and either treated with Pronase to digest residual untranslocated ³⁵S at the cell surface or left untreated as controls. The cells are then lysed, and ³⁵S liberated into the lysis buffer is assayed. The percent translocation is defined as decay per minute (dpm) protected from Pronase/dpm bound to cells ×100. The cell lysate of cells incubated with fusion polypeptides or fragments of domain I that facilitate transmembrane delivery would have higher percent translocation.

Alternatively, green fluorescent protein fused to LFn, LF or smaller fragments of domain I (e.g. LFn-GFP) can be used to assay for membrane translocation capability, as described in N. Kushner, et. al., 2003, Proc. Natl. Acad Sci USA. 100:6652-6657. Briefly, HeLa cells (American Type Culture Collection) are grown on collagen-treated chamber slides (BD Science) to reach ˜80% confluence and incubated with 40 μg/ml purified GFP or LFn-GFP at 37° C. for 1 or 2 h. After washing, GFP fluorescence is compared between GFP and GFP-LFn treated samples. Membrane translocation is evidenced by GFP signal greater in the LFn-GFP-treated cells than in cells treated with GFP alone. Some incubations can also be performed in the presence of 100 μg/ml Texas red-conjugated transferrin (INVITROGEN™ Inc., Molecular Probes) as a marker for the endocytic pathway. For the transferrin experiments, cells are washed four times with cold DMEM and then fixed for 15 min in 4% paraformaldehyde in cold PBS. For antibody labeling, slides are then incubated on ice for 15 min in 50 mM NH₄Cl in PBS and then in PBS containing 0.1% saponin for 20 min on ice. After further washing in PBS, slides are incubated at room temperature for 1 hr in a moisture chamber with PBS containing 4% donkey serum and the following primary antibodies: mouse anti-early endosome antigen 1 (EEA-1) (BD Laboratory) to stain early endosomes, mouse anti-Lamp1 and anti-Lamp2 (Developmental Studies Hybridoma Banks, University of Iowa, Iowa City) to stain late endosomes and lysosome, mouse Ab-1(Oncogene) to stain the Golgi apparatus, mouse anti-mitochondrial antibody from CALBIOCHEM®, and rabbit anti-calreticulin (STRESSGEN® Biotechnologies, Victoria, Canada). Cells are then processed for secondary antibody staining and microscopy. Fusion LFn-GFP that promotes transmembrane delivery would be visualized in the interior of the cell. The antibody markers will further indicate sub-cellular localization of the translocated GFP.

### Zinc Metalloproteinase Activity by FRET Analysis

In some embodiments, one can determine if the composition as disclosed herein has elicited an immune response in the subject to the HIV antigen by determining zinc metalloproteinase activity. Assays of LF peptidolytic activity based on cleavage of the FRET-quenched substrate MAPKKide can be carried out according to a modification of the method of Cummings et al. (2002, Proc. Natl. Acad. Sci. USA 99:6603-6606.). MAPKKide (o-aminobenzoyl [o-ABZ]/2,4-dinitrophenyl [DNP]), a synthetic peptide containing the o-ABZ donor and DNP acceptor groups separated by a cleavage site specific for anthrax LF, was purchased from List Biological Labs. Digestion of MAPKKide by LF was carried out in Dulbecco's phosphate-buffered saline (DPBS) (HyClone, Logan Utah), pH 8.2, as recommended by the manufacturer and was followed in a SpectraMax M2 microplate reader (Molecular Devices, Sunnyvale, Calif.) or in a, LS-5 fluorescence spectrophotometer (Perkin-Elmer, Wellesley, Mass.) using a λ excitation (ex) value of 320 nm and a λ emission (em) value of 420 nm. LF was preincubated with indicated concentrations of putative inhibitors for 10 min at room temperature, and the reaction was initiated by addition of indicated concentrations of the substrate to a 100-μl or 500-μl reaction mixture.

Production of LFn Polypeptide and HIV Antigens using a Baculovirus System

In one embodiment, any of the polypeptides described herein (e.g., HIV antigens and/or LFn polypeptides and fragments thereof) can be produced by expression from any expression vector which are well known by persons of ordinary skill in the art. In some embodiments, the expression vector is a recombinant baculovirus vector. In another embodiment, any of the polypeptides described herein is expressed by an insect cell. In yet another embodiment, any of the polypeptides described herein is isolated from an insect cell. There are several benefits of protein expression with baculovirus in insect cells, including high expression levels, ease of scale-up, production of proteins with posttranslational modifications, and simplified cell growth. Insect cells do not require CO₂ for growth and can be readily adapted to high-density suspension culture for large-scale expression. Many of the post-translational modification pathways present in mammalian systems are also utilized in insect cells, allowing the production of recombinant protein that is antigenically, immunogenically, and functionally similar to the native mammalian protein.

Baculoviruses are DNA viruses in the family Baculoviridae. These viruses are known to have a narrow host-range that is limited primarily to Lepidopteran species of insects (butterflies and moths). The baculovirus Autographa californica Nuclear Polyhedrosis Virus (AcNPV), which has become the prototype baculovirus, replicates efficiently in susceptible cultured insect cells. AcNPV has a double-stranded closed circular DNA genome of about 130,000 base-pairs and is well characterized with regard to host range, molecular biology, and genetics.

Many baculoviruses, including AcNPV, form large protein crystalline occlusions within the nucleus of infected cells. A single polypeptide, referred to as a polyhedrin, accounts for approximately 95% of the protein mass of these occlusion bodies. The gene for polyhedrin is present as a single copy in the AcNPV viral genome. Because the polyhedrin gene is not essential for virus replication in cultured cells, it can be readily modified to express foreign genes. The foreign gene sequence is inserted into the AcNPV gene just 3′ to the polyhedrin promoter sequence such that it is under the transcriptional control of the polyhedrin promoter.

The Baculovirus Expression Vector System (BEVS) is a safe and rapid method for the abundant production of recombinant proteins in insect cells and insects pioneered in the laboratory of Dr. Max D. Summers.

Baculovirus expression systems are powerful and versatile systems for high-level, recombinant protein expression in insect cells. Expression levels up to 500 mg/l have been reported using the baculovirus expression system, making it an ideal system for high-level expression. Recombinant baculoviruses that express foreign genes are constructed by way of homologous recombination between baculovirus DNA and chimeric plasmids containing the gene sequence of interest. Recombinant viruses can be detected by virtue of their distinct plaque morphology and plaque-purified to homogeneity.

Baculoviruses are particularly well-suited for use as eukaryotic cloning and expression vectors. They are generally safe by virtue of their narrow host range which is restricted to arthropods. The U.S. Environmental Protection Agency (EPA) has approved the use of three baculovirus species for the control of insect pests. AcNPV has been applied to crops for many years under EPA Experimental Use Permits.

AcNPV wild type and recombinant viruses replicate in a variety of insect cells, including continuous cell lines derived from the fall armyworm, Spodoptera frugiperda (Lepidoptera; Noctuidae). S. frugiperda cells have a population doubling time of 18 to 24 hours and can be propagated in monolayer or in free suspension cultures.

Recombinant fusion proteins described herein can be produced in insect cells including, but not limited to, cells derived from the Lepidopteran species S. frugiperda. Other insect cells that can be infected by baculovirus, such as those from the species Bombyx mori, Galleria mellanoma, Trichplusia ni, or Lamanthria dispar, can also be used as a suitable substrate to produce recombinant proteins described herein.

Baculovirus expression of recombinant proteins is well known in the art and is described in U.S. Pat. Nos. 4,745,051, 4,879,236, 5,179,007, 5,516,657, 5,571,709 and 5,759,809 which are all incorporated by reference in their entirety. It will be understood by those skilled in the art that the expression system is not limited to a baculovirus expression system. What is important is that the expression system directs the N-glycosylation of expressed recombinant proteins. The recombinant proteins described herein can also be expressed in other expression systems such as Entomopox viruses (the poxviruses of insects), cytoplasmic polyhedrosis viruses (CPV), and transformation of insect cells with the recombinant gene or genes constitutive expression.

The most common expression vector system is from the insect baculovirus A. californica nuclear polyhedrosis virus (AcNPV). AcNPV has a genome of ca. 130 kilobases (kb) of double-stranded, circular DNA and it is the most extensively studied baculovirus. Miller, L. K., J. Virol. 1981, 39:973-976. AcNPV has a biphasic replication cycle and produces a different form of infectious virus during each phase. Between 10 and 24 h postinfection (p.i.), extracellular virus is produced by the budding of nucleocapsids through the cytoplasmic membrane. By 15 to 18 h p.i., nucleocapsids are enveloped within the nucleus and embedded in a paracrystalline protein matrix, which is formed from a single major protein called polyhedrin. In infected S. frugiperda (fall armyworm, Lepidoptera, Noctuidae) cells, AcNPV polyhedrin accumulates to high levels and constitutes 25% or more of the total protein mass in the cell; it may be synthesized in greater abundance than any other protein in a virus-infected eukaryotic cell.

In one embodiment, any of the polypeptides described herein is produced using a Baculovirus Expression Vector System (BEVS), by infecting lepidopteran insect cells with a recombinant baculovirus vector comprising a polynucleotide encoding the polypeptide and culturing the insect cells to produce the polypeptide.

In some embodiments, the Baculovirus Expression Vector System (BEVS) uses lepidopteran insect S. frugiperda cells.

The gene encoding LF has been cloned and sequenced, and has been assigned Genbank accession no. M29081 (Robertson and Leppla, 1986, Gene 44:71 78; Bragg and Robertson, 1989, Gene 81:45 54; see also U.S. Pat. Nos. 5,591,631 and 5,677,274; see generally Leppla, Anthrax Toxins, in Bacterial Toxins and Virulence Factors in Disease (Handbook of natural toxins, Vol. 8., Moss et al., eds., 1995).

The coding DNA sequences are typically cloned into intermediate vectors before transformation into prokaryotic or eukaryotic cells for replication and/or expression. These intermediate vectors are typically cloning plasmids, (e.g. pPUC19, PBLUESCRIPT®-SK) or shuttle vectors that can be propagated in a number of different hosts and to allow more efficient manipulation of DNA (e.g. the pRS YCp and pRS Yip vectors can shuttle between bacteria and Saccharomyces cerevisiae).

To generate influenza virus sequences for expression in a baculovirus system, for example, Virion RNA can be extracted from gradient purified influenza B/Ann Arbor/1/86 and A/Ann Arbor/6/60 (wild-type) viruses by standard methods (Cox et al., 1983, Bulletin of the World Health Organization 61, 143-152). cDNA copies of total viral RNA are prepared by the method of Lapeyre and Amairic (Lapeyre et al., 1985, Gene 37, 215-220) except that first-strand synthesis by reverse transcriptase was primed by using universal influenza type A or B primers complementary to the 3′ untranslated region of virion RNA. The double-stranded cDNA fragment corresponding to influenza genomic RNA segment 5 and 7 (influenza A: 1565 base pairs; influenza B: 1811 base pairs) are isolated from agarose gels, purified, and ligated into the Sma I site of plasmid pUC 8 using standard methods (Maniatis et al., 2001, 3^{rd} edition, Molecular Cloning: A Laboratory Manual. Cold Spring Harbor Laboratory, Cold Spring Harbor, N.Y.) Bacterial colonies (E. coli, HB101) containing recombinant plasmids with NP, M1, or M2 inserts are identified by in situ hybridization (Maniatis et al., (2001). Molecular Cloning: A Laboratory Manual. Cold Spring Harbor Laboratory, Cold Spring Harbor, N.Y.) to ³²P-labeled, oligonucleotide primers with sequences specific for influenza A or B NP, M1 or M2 genes.

LF and LFn coding sequences are described above and can be cloned by one of skill in the art or obtained from existing clones available in the art.

The first step in the production of recombinant proteins from a BEVS is the construction of a recombinant baculovirus vector, either by homologous recombination or by site specific transposition. To obtain a recombinant baculovirus vector by homologous recombination, a baculovirus transfer vector is needed. A baculovirus transfer vector is a temporary vector whose sole purpose is to enable the insertion of foreign coding DNA, under an appropriate gene promoter, into the baculovirus genome at a site that would not affect normal viral replication. The baculovirus transfer vector comprises a portion of the baculovirus genomic sequence that spans the intended insertion site of the foreign coding DNA. The most common regions contain the polyhedrin or p10 gene. Both are dispensible for viral replication in cell culture and insect larvae, and the production of infectious extracellular virus. Both proteins are highly expressed at the very late phase of viral replication and effect high level of transcription of the foreign gene when inserted back into the viral genome. A typical baculovirus transfer vector comprises a promoter, a transcriptional terminator, and most often native viral sequences and regions flanking both sides of the promoter that are homologous to the target genes in the viral genome. The region between the promoter and the transcriptional terminator can have multiple restriction enzyme digestion sites for facilitating cloning of the foreign coding sequence, in this instance, the coding DNA sequence for an LF polypeptide, e.g., an LFn polypeptide and an HIV antigen. Additional sequences can be included, e.g., signal peptides and/or tag coding sequences, such as His-tag, MAT-Tag, FLAG tag, recognition sequence for enterokinase, honeybee melittin secretion signal, beta-galactosidase, glutathione S-transferase (GST) tag upstream of the MCS for facilitating the secretion, identification, proper insertion, positive selection of recombinant virus, and/or purification of the recombinant protein. Subsequent to the construction of the baculovirus transfer vector, it is mixed with AcNPV viral DNA and co-transfected into insect cells to establish an infection. The native polyhedrin gene is removed by a double-cross over homologous recombination event and replaced by the foreign coding sequence to be expressed in the insect cells. Inactivation of the polyhedrin gene by deletion or by insertion results in mutants that do not produce occlusions in infected cells. These occlusion-negative viruses form plaques that are different from plaques produced by wild-type viruses, and this distinctive plaque morphology is useful as a means to screen for recombinant viruses.

A good number of baculovirus transfer vectors and the corresponding appropriately modified host cells are commercially available, for example, pAcGP67, pAcSECG2TA, pVL1392, pVL1393, pAcGHLT, and pAcAB4 from BD Biosciences; pBAC-3, pBAC-6, pBACgus-6, and pBACsurf-1 from NOVAGEN®, and pPolh-FLAG and pPolh-MAT from SIGMA ALDRICH®. One skilled in the art would be able to clone and ligate the coding region of the B. anthracis lethal factor N-terminal (LFn) portion with the coding region of a HIV antigen polypeptide or fragment thereof to construct a chimeric coding sequence for a fusion polypeptide comprising LFn and the HIV antigen polypeptide or fragment thereof using specially designed oligonucleotide probes and polymerase chain reaction (PCR) methodologies that are well known in the art. One skilled in the art would also be able to clone and ligate the chimeric coding sequence for a fusion protein into a selected baculovirus transfer vector. The coding sequences of LFn and the HIV antigen polypeptide or fragment thereof should be ligated in-frame and the chimeric coding sequence should be ligated downstream of the promoter, and between the promoter and the transcription terminator. Subsequent to that, the recombinant baculovirus transfer vector is transfected into regular cloning Escherichia coli, such as XL1B1ue. Recombinant E. coli harboring the transfer vector DNA is then selected by antibiotic resistance to remove any E. coli harboring non-recombinant plasmid DNA. The selected transformant E. coli are grown and the recombinant vector DNA is subsequently purified for transfection into S. frugiperda (SF) cells.

As an example, the oligonucleotide 5′-GGAGGAACATATGGCGGGCGGTCATGGTGATG-3′ (SEQ. ID. No. 19) can be used to introduce an NdeI site and serve as a forward primer in the amplification of the coding DNA sequence for LFn-(amino acids 1-263) and the oligonucleotide 5′-CTAGGATCCTTACCGTTGATCTTTAAGTTCTTCC-3′ (SEQ. ID. No. 20) can be used to introduce a BamHI site and act as the reverse primer. PCR amplification is performed using the cDNA template according to GenBank Accession No. M29081. The forward primers for LFn-(28-263), LFn-(33-263), LFn-(37-263), LFn-(40-263), and LFn-(43-263) can be designed accordingly permit the PCR amplification of the coding sequence of the appropriate truncated LFn and also introduce an NdeI site.

As an example, for cloning the full-length NP, the oligonucleotide CTAGAAGTCC ATGGCGTCCCAAGGCACCAAACGG (SEQ. ID. No. 21) can be used to introduce a BamHI site and 5′-CTAGAGCTCATTGTCGTACTCTTCTGCATTGTC-3′ can be used to introduce a XhoI site (SEQ. ID. No. 22)

The common BamHI site at the end of the amplified coding sequence of LFn and at the beginning of the amplified coding sequence of NP facilitates the ligation of the two separate amplified coding sequences into a chimeric or fusion coding sequence. The ligation of the two separate amplified coding sequences should be such that NP is in frame with LFn and there is no translation stop codon around the ligation site. The fusion coding sequence can then be digested with NdeI and XhoI and ligated into a selected baculovirus transfer vector that has NdeI and XhoI sites with the appropriate orientation. The newly constructed baculovirus transfer vector can be transformed into E. coli DHS. E. coli transformants can be screened by digestion and verified by sequencing. After that, the baculovirus transfer vector can be isolated for co-transfection into insect cells for homologous recombination. Similar approaches can obviously be taken for cloning other influenza antigen sequences.

To obtain a recombinant baculovirus vector by site specific transposition, e.g. with Tn7 to insert foreign genes into bacmid DNA propagated in E. coli., INVITROGEN™ Inc. provides the pFASTBAC™ plasmid and bacmid containing DH10BAC™ competent E. coli for constructing a recombinant baculovirus vector by site specific transposition. The coding sequence is cloned into a pFASTBAC™ plasmid and the recombinant plasmid is transformed into an DH10BAC™ competent E. coli harboring bacmid, a baculovirus shuttle vector, with a mini-attTn7 target site and a helper plasmid. The mini-attTn7 element on the pFASTBAC™ plasmid can transpose to the mini-attTn7 target site on the bacmid in the presence of transposition proteins provided by the helper plasmid. Colonies containing recombinant bacmids are identified by antibiotics selection and by blue/white screening, since the transposition results in the disruption of the LacZα gene that is flanked by the mini-attTn7 target site on the bacmid. The bacmid is then harvested for transfection of insect cells.

In one embodiment, a fusion polypeptide described herein has a spacer peptide, e.g., a 14-residue spacer (GSPGISGGGGGILE) (SEQ. ID. No. 23) separating the LF polypeptide (e.g., an LFn polypeptide) from the influenza polypeptide. The coding sequence of such a short spacer can be constructed by annealing a complementary pair of primers. One of skill in the art can design and synthesize oligonucleotides that will code for the selected spacer. Spacer peptides should generally have non-polar amino acid residues, such as glycine and proline.

In some instances, specific site-directed mutagenesis of the chimeric coding sequence in the baculovirus transfer vector can be performed to create specific amino acid mutations and substitutions to further promote transmembrane delivery, protein expression or protein folding. An example of an amino acid substitution include glutamate for aspartate. Site-directed mutagenesis can be carried out, e.g., using the QUIKCHANGE® site-directed mutagenesis kit from Stratagene according to manufacture's instructions or any methods known in the art.

Standard viral DNA is used to co-transfect S. frugiperda (SF) cells. Putative recombinant viruses containing the recombinant molecules are isolated from the virus produced in these transfected monolayers. Because the polyhedrin structural gene has been removed, plaques containing the recombinant viruses can be easily identified since they lack occlusion bodies. Confirmation that these recombinants contain the desired chimeric coding sequence is established by methods well known in the art, such as hybridization with specific gene probes, plaque assays, and end point dilution.

A preferred host cell line for protein production from recombinant baculoviruses described herein is Sf900+. Another preferred host cell line for protein production from recombinant baculoviruses is Sf9. Sf900+and Sf9 are non-transformed, non-tumorigenic continuous cell lines derived from the fall armyworm, S. frugiperda (Lepidoptera; Noctuidae).

Sf900+ and Sf9 cells are propagated at 28±2° C. without carbon dioxide supplementation. The culture medium used for Sf9 cells is TNMFH, a simple mixture of salts, vitamins, sugars and amino acids, supplemented with 10% fetal bovine serum. Aside from fetal bovine serum, no other animal derived products (i.e, trypsin, etc.) are used in cell propagation. Serum free culture medium (available as Sf900 culture media, GIBCO® BRL, Gaithersburg, Md.) can also be used to grow Sf9 cells and is preferred for propagation of Sf900+cells. Sf9 cells have a population doubling time of 18-24 hours and can be propagated in monolayer or in free suspension cultures. S. frugiperda cells have not been reported to support the replication of any known mammalian viruses.

Plaque assays of baculovirus transfected monolayers SF cells are well known in the art. Below is a standard protocol.

Reagents needed: Grace's Insect Medium, 2× (e.g. BD Biosciences GIBCO® #11667), fetal bovine serum (Heat Inactivated), (e.g. BD Biosciences GIBCO® #16140), 3% SEAPLAQUE® or other low-melting agarose in ddH₂O, sterile water, 50 ml sterile conical screw-top tubes, and 37° C. water bath microwave

Step one: Prepare infected monolayer of cells
1. Grow a suspension culture of Sf9 cells to a density of less than 3×10⁶.
2. Dilute this culture to a density of 5 to 6×10⁵.
3. For a 6-well culture dish, transfer 2 ml of this cell suspension to each well. For 6 cm dishes, double all volumes in this protocol. Scale by surface area. This cell number will depend somewhat on the cell line and can be adjusted up or down according to your results. If there is no confluent monolayer by day 3, increase the cell number. There should be space available on Day 2.
4. Let the cells settle for at least 30 min to ensure the cells are firmly attached.
5. Meanwhile, dilute the plaque virus to 1 ml aliquots at dilutions of 10⁻⁴, 10⁻⁵, 10⁻⁶ and 10⁻⁷.
6. After SF cells have attached well to the plates, aspirate off the media.
7. Quickly add 1 ml of diluted virus to each well of the 6-well plate.
8. Transfer the plates to a rocking platform and slowly rock for at least two hours, four hours is preferred, though after this the benefit diminishes substantially.

Step Two. Prepare the overlay agarose just before use.
9. Mix as follows: 1 part 2× Grace's Medium supplemented with 20% Fetal Calf Serum and 1 part 3% SEAPLAQUE® Agarose in double distilled water (ddH₂O).
10. Melt the agarose completely.
11. Allow the agarose to cool slightly, to near 70° C. and then aliquot 20 nil to each 50 nil conical tube.
12. Add 20 nil of room temp or warmer 2× Graces/FCS to each 20 nil aliquot of agarose, then place in a 35-37° C. water bath.
13. Remove the overlay agarose from the water bath one tube at a time and check the temperature. Let it cool to at least 38° C., but preferably less than 37° C.

Step Three. Overlay agarose onto infected cell monolayer
14. When ready, aspirate all the medium off.
15. Return the plate to level and quickly add approximately 3 ml of molten overlay mix to each well by allowing it to slide down the far wall of the well and onto the plate.
16. After overlaying the cells let the plates sit level in the hood for 30 or so minutes to dry a bit and solidify.
17. Place in a 27° C., 98% humidity controlled incubator for at least 3 days.

Step Four. Staining the Plates
18. Prepare a solution of 1% Neutral red.
19. Prepare an overlay agarose solution as above, but only prepare 1 ml for each assay.
20. Add 1/100th volume of the 1% Neutral Red solution to the molten agarose (e.g. 100 microliters to 10 ml).
21. Add approximately 1 ml of the Red Agarose to each well of a 6-well dish. Be sure the plate is level until the agarose sets up.
22. Add enough Red Agarose to cover the surface evenly.
23. Return the plate to the incubator for at least 4 hours. After several hours the plaques will begin to appear as clear spots among stained cells.
24. The plates can be left overnight before counting.
25. Controls can verify that longer incubations do not give higher titer results with the medium and cells used.

In one embodiment, the positive plaques can be identified by end point dilution assay (EPDA). A 96-well plate EPDA can be used to replace the plaque assay and plaque purification as a method for either determining viral titer or identifying and purifying recombinant virus. A modified 12-well plate EPDA can be used as a routine method for determining viral titer; it is useful for estimating the efficiency of the initial co-transfection, identifying infected cells, approximating viral titers, and amplifying viral titer. In the 12-well EPDA, individual wells containing equal amounts of insect cells are inoculated with 100, 10, 1 or 0 μl aliquots of the original transfection supernatant, wild-type virus, or recombinant Xy1E positive control viral supernatant. A visual comparison between cells in wells inoculated with 100, 10, 1 and 0 μl is used to estimate the viral titer.

For example, if cells receiving 100 μl of the initial co-transfection supernatant look infected in the EPDA, but cells receiving 10, 1 and 0 μl do not, then it is likely that the viral titer is low and should be amplified to produce a high titer stock. If wells receiving 100 μl of the original co-transfection supernatant look similar to those receiving 0 μl, it is likely that the original co-transfection did not result in a significant viral titer and must be repeated. When assaying the efficiency of a co-transfection or estimating the titer of a virus stock, if the EPDA shows a 10 fold decrease in the number of infected cells between dilutions, amplify the virus once or twice more to generate a high titer stock for protein production. However, if all three wells (100, 10, 1 μl) show equal signs of infection, the viral titer is high, ˜2×10⁸ plaque-forming units (pfu)/ml. High titer recombinant virus stocks are used for infection of cells at optimal multiplicity of infection (MOI=# of virus/# of cells) resulting in maximum protein production.

If the EPDA is used as an amplification step to generate a high titer stock, cross contamination between wells containing different viruses, e.g., the highly infectious wild-type virus used as a positive control, can be avoided by using separate 12 well plates.

EPDA controls are recommended. The recombinant virus from a pVL1392-Xy1E transfection is a particularly useful positive control. Infected cells producing the XylE protein turn yellow in the presence of catechol and are easily identifiable. An example of a protocol for EPDA follows:

Protocol

1. Dilute log-phase Sf9 cells (with greater than 98% viability) to 1×105 cells/ml with fresh TNM-FH medium. Seed 1×105 Sf9 cells per well on a 12-well plate (BD Falcon™, Cat. No. 353043). Allow cells to attach firmly, approximately 10 min. Confirm 30% confluency by visualization on a light microscope. Replace medium with 1 ml fresh TNM-FH.

2. Add 100, 10, 1 and 0 μl of the recombinant virus supernatant obtained 5 days after the start of co-transfection (or other virus stock), to separate wells. Do the same for the positive control, e.g., pVL1392-XylE supernatant.

3. Incubate the cells at 27° C. for three days. Examine the cells for signs of infection.

4. A successful transfection should result in uniformly large infected cells in the 100, 10, and 1 μl experimental wells.

5. If only the 100 μl and 10 μl wells seem to have infected cells and the 1 μl well looks more like the control, the titer of virus supernatant is low. Amplify the virus an additional time before proceeding with protein production.

Protein production can be analyzed by western blot analysis (if antibodies are available) or by Coomassie blue-stained SDS-PAGE gel by harvesting cells from the 100 μl well and lysing in appropriate lysing buffer.

The virus supernatant from the 100 μl well can be kept as the first viral amplification stock, however care should be taken to avoid cross-contamination between wells containing different virus.

To further purify the virus population, a plaque assay purification of the co-transfection supernatant can be performed using the approximate titer obtained from the EPDA.

Once recombinant baculoviral vectors that express the proteins are established, then the virus can be amplified and purified for infection of SF cells.

Purification of Virus. Viral particles produced from the first passage are purified from the media using a known purification method such as sucrose density gradient centrifugation. For example, virus is harvested 24-48 hours post infection by centrifuging media of infected cells. The resulting viral pellet is resuspended in buffer and centrifuged through a buffered sucrose gradient. The virus band is harvested from the 40-45% sucrose region of the gradient, diluted with buffer and pelleted by centrifugation at 100,000×g. The purified virus pellet is resuspended in buffer and stored at −70° C. or used in large scale infection of cells for protein production.

The infection process, including viral protein synthesis, viral assembly and partial cell lysis can be complete by approximately 72 hours post-infection. This can be protein dependent and thus can occur earlier or later. The proteins produced in infected cells can be radiolabeled with ³⁵S-methionine, ³H-leucine, or ³H-mannose and both cell-associated and cell-free polypeptides can be analyzed by electrophoresis on polyacrylamide gels to determine their molecular weight. The expression of these products can also be examined at different times post-infection, prior to cell lysis.

Immunological identification of expressed fusion polypeptides can be performed, e.g., by either direct immunoprecipitation or by Western blots. For Western blots, cell-associated proteins or the proteins in the media are separated on SDS polyacrylamide gels, transferred onto nitrocellulose or nylon filters, and identified with antiserum to the LF polypeptide or HIV antigen proteins or to the polyhedrin. Specifically bound antibody is detected by incubating the filters with ¹²⁵I-labeled protein A or enzyme conjugated anti-antibody, and followed by exposure to X-ray film at −80° C. with intensifying screens or colorimetic reaction with enzyme substrate.

Having confirmed the identity of the expressed fusion polypeptides, the next step is to purify the proteins for uses and compositions described herein, e.g. evaluation for use as vaccines (e.g. protective/prophylactic or therapeutic vaccination) or screening agents. If the fusion polypeptides described herein are designed with secretion signal peptides, the encoded polypeptides are often released into the cell culture medium. Media from these infected cells can be concentrated and the proteins purified using standard methods. Salt precipitation, sucrose gradient centrifugation and chromatography, high or fast pressure liquid chromatography (HPLC or FPLC) can be used because these methods allow rapid, quantitative and large scale purification of proteins, and do not denature expressed products.

The efficiency of synthesis of the desired gene product is dependent on multiple factors including: (1) the choice of an expression vector system; (2) the number of gene copies that will be available in the cells as templates for the production of mRNA; (3) the promoter strength; (4) the stability and structure of the mRNA; (5) the efficient binding of ribosomes for the initiation or translation; (6) the properties of the protein product, such as, the stability of the gene product or lethality of the product to the host cells; and (7) the ability of the system to synthesize and export the protein from the cells, thus simplifying subsequent analysis, purification and use.

Purification of recombinant influenza proteins expressed in a BEVS is known in the art, for example, in U.S. Pat. Nos. 5,290,686, 5,976,552, 7,399,840 and U.S. Patent Application No. 2008/0008725, all of which are incorporated herein by reference in their entirety.

### Production of Fusion Polypeptide Using Other Expression Systems

The fusion polypeptides described herein can all be synthesized and purified by protein and molecular methods that are well known to one skilled in the art. Preferably molecular biology methods and recombinant heterologous protein expression systems are used. For example, recombinant protein can be expressed in mammalian, insect, yeast, or plant cells.

Some examples of recombinant cloning and truncation of LF, LFn, their expression, and specific site mutations and insertions are described, for example, in WO/2002/079417, WO/2008/048289, U.S. Patent Application No. 2004/0166120, Huyen Cao, et. al., 2002, J. Infectious Diseases; 185:244-251; N. Kushner, et. al., 2003, Proc. Natl. Acad. Sci. USA. 100:6652-6657; Ballard, J. D., et. al., 1996, Proc. Natl. Acad. Sci. USA 93:12531-12534; and Goletz, T. J. et al., 1997, Proc. Natl. Acad. Sci. USA 94:12059-12064, all of which are incorporated herein by reference in their entirety. Approaches similar to those described in these references can be used to produce the fusion polypeptides as described herein.

In one embodiment, provided herein is an isolated polynucleotide encoding a fusion polypeptide or a non-fusion polypeptide described herein. Conventional polymerase chain reaction (PCR) cloning techniques can be used to construct a chimeric or fusion coding sequence encoding a fusion polypeptide as described herein. A coding sequence can be cloned into a general purpose cloning vector such as pUC19, pBR322, pBLUESCRIPT® vectors (STRATAGENE® Inc.) or pCR TOPO® from INVITROGEN™ Inc. The resultant recombinant vector carrying the nucleic acid encoding a polypeptide as described herein can then be used for further molecular biological manipulations such as site-directed mutagenesis to create a variant fusion polypeptide as described herein or can be subcloned into protein expression vectors or viral vectors for protein synthesis in a variety of protein expression systems using host cells selected from the group consisting of mammalian cell lines, insect cell lines, yeast, bacteria, and plant cells.

Each PCR primer should have at least 15 nucleotides overlapping with its corresponding templates at the region to be amplified. The polymerase used in the PCR amplification should have high fidelity such as STRATAGENE® PfuULTRA® polymerase for reducing sequence mistakes during the PCR amplification process. For ease of ligating several separate PCR fragments together, for example in the construction of a fusion polypeptide, and subsequently inserting into a cloning vector, the PCR primers should also have distinct and unique restriction digestion sites on their flanking ends that do not anneal to the DNA template during PCR amplification. The choice of the restriction digestion sites for each pair of specific primers should be such that the fusion polypeptide coding DNA sequence is in-frame and will encode the fusion polypeptide from beginning to end with no stop codons. At the same time the chosen restriction digestion sites should not be found within the coding DNA sequence for the fusion polypeptide. The coding DNA sequence for the intended polypeptide can be ligated into cloning vector pBR322 or one of its derivatives, for amplification, verification of fidelity and authenticity of the chimeric coding sequence, substitutions/or specific site-directed mutagenesis for specific amino acid mutations and substitutions in the polypeptide.

Alternatively the coding DNA sequence for the polypeptide can be PCR cloned into a vector using for example, INVITROGEN™ Inc.'s TOPO® cloning method comprising topoisomerase-assisted TA vectors such as pCR®-TOPO, pCR®-Blunt II-TOPO, pENTR/D-TOPO®, and pENTR/SD/D-TOPO®. Both pENTR/D-TOPO®, and pENTR/SD/D-TOPO® are directional TOPO entry vectors which allow the cloning of the DNA sequence in the 5′→3′ orientation into a GATEWAY® expression vector. Directional cloning in the 5′→3′ orientation facilitates the unidirectional insertion of the DNA sequence into a protein expression vector such that the promoter is upstream of the 5′ ATG start codon of the fusion polypeptide coding DNA sequence, enabling promoter driven protein expression. The recombinant vector carrying the coding DNA sequence for the fusion polypeptide can be transfected into and propagated in general cloning E. coli such as XL1Blue, SURE® (STRATAGENE®) and TOP-10 cells (INVITROGEN™ Inc.).

Standard techniques known to those of skill in the art can be used to introduce mutations (to create amino acid substitutions in the polypeptide sequence of the fusion polypeptide described herein, e.g., in the LFn polypeptide, i.e. SEQ. ID. No. 3 or 4 or 5) in the nucleotide sequence encoding the fusion polypeptide described herein, including, for example, site-directed mutagenesis and PCR-mediated mutagenesis. Preferably, the variant fusion polypeptide has less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the fusion polypeptides described herein.

Certain silent or neutral missense mutations can also be made in the DNA coding sequence that do not change the encoded amino acid sequence or the capability to promote transmembrane delivery. These types of mutations are useful to optimize codon usage, or to improve recombinant protein expression and production.

Specific site-directed mutagenesis of a coding sequence for the fusion polypeptide in a vector can be used to create specific amino acid mutations and substitutions. Site-directed mutagenesis can be carried out using, e.g. the QUIKCHANGE® site-directed mutagenesis kit from Stratagene according to the manufacturer's instructions.

In one embodiment, described herein are expression vectors comprising the coding DNA sequence for the polypeptides described herein for the expression and purification of the recombinant polypeptide produced from a protein expression system using host cells selected from, e.g., mammalian, insect, yeast, or plant cells. The expression vector should have the necessary 5′ upstream and 3′ downstream regulatory elements such as promoter sequences, ribosome recognition and TATA (SEQ. ID. No. 33) box, and 3′ UTR AAUAAA (SEQ. ID. No. 34) transcription termination sequence for efficient gene transcription and translation in its respective host cell. The expression vector is, preferably, a vector having the transcription promoter selected from a group consisting of CMV (cytomegalovirus) promoter, RSV (Rous sarcoma virus) promoter, β-actin promoter, SV40 (simian virus 40) promoter and muscle creatine kinase promoter, and the transcription terminator selected from a group consisting of SV40 poly (A) and BGH terminator; more preferably, an expression vector having the early promoter/enhancer sequence of cytomegalovirus and the adenovirus tripartite leader/intron sequence and containing the replication orgin and poly (A) sequence of SV40. The expression vector can have additional sequence such as 6×-histidine, V5, thioredoxin, glutathione-5-transferase, c-Myc, VSV-G, HSV, FLAG, maltose binding peptide, metal-binding peptide, HA and “secretion” signals (Honeybee melittin, α-factor, PHO, Bip), which are incorporated into the expressed fusion polypeptide. In addition, there can be enzyme digestion sites incorporated after these sequences to facilitate enzymatic removal of them after they are not needed. These additional sequences are useful for the detection of fusion polypeptide expression, for protein purification by affinity chromatography, enhanced solubility of the recombinant protein in the host cytoplasm, and/or for secreting the expressed fusion polypeptide out into the culture media or the spheroplast of the yeast cells. The expression of the fusion polypeptide can be constitutive in the host cells or it can be induced, e.g., with copper sulfate, sugars such as galactose, methanol, methylamine, thiamine, tetracycline, infection with baculovirus, and (isopropyl-beta-D-thiogalactopyranoside) IPTG, a stable synthetic analog of lactose.

In another embodiment, the expression vector comprising a polynucleotide described herein is a viral vector, such as adenovirus, adeno-associated virus (AAV), retrovirus, and lentivirus vectors, among others. Recombinant viruses provide a versatile system for gene expression studies and therapeutic applications.

The polypeptides described herein can be expressed in a variety of expression host cells e.g., yeasts, mammalian cells, insect cells and plant cells such as Chlamadomonas, or even in cell-free expression systems. From the cloning vector, the nucleic acid can be subcloned into a recombinant expression vector that is appropriate for the expression of fusion polypeptide in mammalian, insect, yeast, or plant cells or a cell-free expression system such as a rabbit reticulocyte expression system. Some vectors are designed to transfer coding nucleic acid for expression in mammalian cells, insect cells and year in one single recombination reaction. For example, some of the GATEWAY® (INVITROGEN™ Inc.) destination vectors are designed for the construction of baculovirus, adenovirus, adeno-associated virus (AAV), retrovirus, and lentiviruses, which upon infecting their respective host cells, permit heterologous expression of fusion polypeptides in the appropriate host cells. Transferring a gene into a destination vector is accomplished in just two steps according to manufacturer's instructions. There are GATEWAY® expression vectors for protein expression in insect cells, mammalian cells, and yeast. Following transformation and selection in E. coli, the expression vector is ready to be used for expression in the appropriate host.

Examples of other expression vectors and host cells are the strong CMV promoter-based pcDNA3.1 (INVITROGEN™ Inc.) and pCINEO vectors (Promega) for expression in mammalian cell lines such as CHO, COS, HEK-293, Jurkat, and MCF-7; replication incompetent adenoviral vector vectors pADENO-X™, pAd5F35, pLP-ADENO™-X-CMV (CLONTECH®), pAd/CMV/V5-DEST, pAd-DEST vector (INVITROGEN™ Inc.) for adenovirus-mediated gene transfer and expression in mammalian cells; pLNCX2, pLXSN, and pLAPSN retrovirus vectors for use with the RETRO-X™ system from Clontech for retroviral-mediated gene transfer and expression in mammalian cells; pLenti4/V5-DEST™, pLenti6/V5-DESTT™, and pLenti6.2N5-GW/lacZ (INVITROGEN™) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells; BACpak6 baculovirus (Clontech) and pFASTBAC™ HT (INVITROGEN™) for the expression in S. frugiperda 9 (Sf9), Sf11, Tn-368 and BTI-TN-5B4-1 insect cell lines; pMT/BiP/V5-His (INVITROGEN™) for the expression in Drosophila Schneider S2 cells; Pichia expression vectors pPICZα, pPICZ, pFLDα and pFLD (INVITROGEN™) for expression in Pichia pastoris and vectors pMETα and pMET for expression in P. methanolica; pYES2/GS and pYD1 (INVITROGEN™) vectors for expression in yeast S. cerevisiae. Recent advances in the large scale expression heterologous proteins in Chlamydomonas reinhardtii are described by Griesbeck C. et. al., 2006 Mol. Biotechnol. 34:213-33 and Fuhrmann M., 2004, Methods Mol. Med. 94:191-5. Foreign heterologous coding sequences are inserted into the genome of the nucleus, chloroplast and mitochondria by homologous recombination. The chloroplast expression vector p64 carrying the most versatile chloroplast selectable marker aminoglycoside adenyl transferase (aadA), which confer resistance to spectinomycin or streptomycin, can be used to express foreign protein in the chloroplast. The biolistic gene gun method can be used to introduce the vector in the algae. Upon its entry into chloroplasts, the foreign DNA is released from the gene gun particles and integrates into the chloroplast genome through homologous recombination.

In some embodiments, the fusion polypeptides described herein are expressed from viral infection of mammalian cells. The viral vectors can be, for example, adenovirus, adeno-associated virus (AAV), retrovirus, and lentivirus. A simplified system for generating recombinant adenoviruses is presented by He et al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pADEASY-1 of STRATAGENE®'s ADEASY™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

Recombinant lentivirus has the advantage of delivery and expression of fusion polypeptides in dividing and non-dividing mammalian cells. The HIV-1 based lentivirus can effectively transduce a broader host range than the Moloney Leukemia Virus (MoMLV)-based retroviral systems. Preparation of the recombinant lentivirus can be achieved using, for example, the pLenti4/V5-DEST™, pLenti6/V5-DEST™ or pLenti vectors together with VIRAPOWER™ Lentiviral Expression systems from INVITROGEN™ Inc.

Recombinant adeno-associated virus (rAAV) vectors are applicable to a wide range of host cells including many different human and non-human cell lines or tissues. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

Large scale preparation of AAV vectors is made by a three-plasmid cotransfection of a packaging cell line: AAV vector carrying the coding nucleic acid, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors can be purified by two different methods depending on the serotype of the vector. AAV2 vector is purified by the single-step gravity-flow column purification method based on its affinity for heparin (Auricchio, A., et. al., 2001, Human Gene therapy 12:71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

The polypeptides described herein can be expressed and purified by a variety methods known to one skilled in the art, for example, the fusion polypeptides described herein can be purified from any suitable expression system. Fusion polypeptides can be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al. supra).

A number of procedures can be employed when recombinant proteins are purified. For example, proteins having established molecular adhesion properties can be reversibly fused to the protein of choice. With the appropriate ligand, the protein can be selectively adsorbed to a purification column and then freed from the column in a relatively pure form. The fused protein is then removed by enzymatic activity. Finally, the protein of choice can be purified using affinity or immunoaffinity columns.

After the protein is expressed in the host cells, the host cells can be lysed to liberate the expressed protein for purification. Methods of lysing the various host cells are featured in “Sample Preparation-Tools for Protein Research” EMD Bioscience and in the Current Protocols in Protein Sciences (CPPS). A preferred purification method is affinity chromatography such as metal-ion affinity chromatograph using nickel, cobalt, or zinc affinity resins for histidine-tagged fusion polypeptides. Methods of purifying histidine-tagged recombinant proteins are described by Clontech using their TALON® cobalt resin and by NOVAGEN® in their pET system manual, 10th edition. Another preferred purification strategy is immuno-affinity chromatography, for example, anti-myc antibody conjugated resin can be used to affinity purify myc-tagged fusion polypeptides. When appropriate protease recognition sequences are present, fusion polypeptides can be cleaved from the histidine or myc tag, releasing the fusion polypeptide from the affinity resin while the histidine-tags and myc-tags are left attached to the affinity resin.

Standard protein separation techniques for purifying recombinant and naturally occurring proteins are well known in the art, e.g. solubility fractionation, size exclusion gel filtration, and various column chromatography.

Solubility fractionation: Often as an initial step, particularly if the protein mixture is complex, an initial salt fractionation can separate many of the unwanted host cell proteins (or proteins derived from the cell culture media) from the protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size exclusion filtration: The molecular weight of the protein of choice can be used to isolate it from proteins of greater and lesser size using ultrafiltration through membranes of different pore size (for example, AMICON® or MILLIPORE® membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column chromatography: The protein of choice can also be separated from other proteins on the basis of its size, net surface charge, hydrophobicity, and affinity for ligands. In addition, antibodies raised against recombinant or naturally occurring proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech). For example, LFn can be purified using a PA63 heptamer affinity column (Singh et al., 1994, J. Biol. Chem. 269:29039-29046).

In some embodiments, a combination of purification steps comprising, for example: (i) anion exchange chromatography, (ii) hydroxyapatite chromatography, (iii) hydrophobic interaction chromatography, and (iv) size exclusion chromatography can be used to purify the fusion polypeptides described herein.

Cell-free expression systems are also contemplated. Cell-free expression systems offer several advantages over traditional cell-based expression methods, including the easy modification of reaction conditions to favor protein folding, decreased sensitivity to product toxicity and suitability for high-throughput strategies such as rapid expression screening or large amount protein production because of reduced reaction volumes and process time. The cell-free expression system can use plasmid or linear DNA. Moreover, improvements in translation efficiency have resulted in yields that exceed a milligram of protein per milliliter of reaction mix. Commercially available cell-free expression systems include the TNT coupled reticulocyte lysate Systems (Promega) which uses rabbit reticulocyte-based in-vitro system.

In some embodiments of the present invention may be defined in any of the following numbered paragraphs:

1. The use of a pharmaceutical composition comprising a pharmaceutically acceptable carrier and an antigen preparation, the antigen preparation comprising a HIV polypeptide or fragment thereof and at least residues 34-288 of the N-terminal Bacillus anthracis Lethal Factor (LFn) polypeptide for augmenting the treatment of an HIV anti-retroviral therapy in a subject.
2. The use of the composition of paragraph 1, wherein the HIV polypeptide or fragment thereof is fused the LFn polypeptide.
3. The use of the composition of paragraphs 1 or 2, wherein said composition is administered to a subject in combination with traditional antiretroviral therapies.
4. The use of the composition of any of paragraphs 1 to 3, wherein said composition is administered on a periodic basis to the subject.
5. The use of the composition of any of paragraphs 1 to 4, wherein said composition is administered to the subject at least yearly.
6. The use of the composition of any of paragraphs 1 to 5, wherein said composition is administered to the subject at least twice a year.
7. The use of the composition of any of paragraphs 1 to 6, wherein said composition is administered to the subject at least quarterly.
8. The use of the composition of any of paragraphs 1 to 7, wherein said composition is administered to the subject at least monthly.
9. The use of the composition of any of paragraphs 1 to 8, wherein said composition is administered to the subject more than once a month.
10. The use of the composition of any of paragraphs 1 to 9, further comprising an adjuvant.
11. The use of the composition of any of paragraphs 1 to 10, wherein said adjuvant is selected from the group consisting of QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59.
12. The use of the composition of any of paragraphs 1 to 11, wherein said LFn polypeptide is a conservative substitution variant thereof, that promotes transmembrane delivery to the cytosol of an intact cell.
13. The use of the composition of any of paragraphs 1 to 12, wherein said LFn polypeptide is N-glycosylated.
14. The use of the composition of any of paragraphs 1 to 13, wherein said LFn polypeptide comprises at least the 60 carboxy-terminal amino acids of SEQ. ID. No. 3, or a conservative substitution variant thereof.
15. The use of the composition of any of paragraphs 1 to 14, wherein said LFn polypeptide comprises at least the 80 carboxy-terminal amino acids of SEQ. ID. No. 3, or a conservative substitution variant thereof.
16. The use of the composition of any of paragraphs 1 to 15, wherein said LFn polypeptide comprises at least the 104 carboxy-terminal amino acids of SEQ. ID. No. 3, or a conservative substitution variant thereof.
17. The use of the composition of any of paragraphs 1 to 16, wherein said LFn polypeptide comprises the amino acid sequence corresponding to SEQ. ID. No. 5, or a conservative substitution variant thereof.
18. The use of the composition of any of paragraphs 1 to 17, wherein said LFn polypeptide does not bind B. anthracis protective antigen protein.
19. The use of the composition of any of paragraphs 1 to 18, wherein said LFn polypeptide substantially lacks the amino acids 1-33 of SEQ. ID. No. 3.
20. The use of the composition of any of paragraphs 1 to 19, wherein said LFn polypeptide consists of SEQ. ID. No. 5, or a conservative substitution variant thereof.
21. The use of the composition of any of paragraphs 1 to 20, wherein said LFn polypeptide is fused to said HIV polypeptide or a fragment thereof of at least 15 amino acids.
22. The use of the composition of any of paragraphs 1 to 21, wherein the HIV polypeptide and/or LFn polypeptide is expressed and isolated from a baculovirus expression system.
23. The use of the composition of any of paragraphs 1 to 22, wherein the at least one antiretroviral therapy is selected from any or a combination from the group consisting of: stem cell therapy, Tenofovir, Lamivudine, Zidovudine, Abacavir, zidovudine AZT, (S)-6-chloro-4-(cyclopropylethynyl)-1,4-dihydro-4-(trifluoromethyl)-2H-3,11-Cyclopropyl-5,11-dihydro-4-methyl-6H-dipyrido[3,2-b: 2′,3′-e][1,4]diazepin-6-one, or derivatives thereof.
24. The use of the composition of any of paragraphs 1 to 24, wherein the subject is a human subject.
25. The use of the composition of any of paragraphs 1 to 25, wherein the subject undergoing a HIV anti-retroviral therapy can reduce their anti-retroviral therapy regimen.
26. The use of the composition of any of paragraphs 1 to 26, wherein the subject undergoing a HIV anti-retroviral therapy can miss an occasional dose of their anti-retroviral therapy regimen.
27. The use of the composition of any of paragraphs 1 to 25, wherein the subject undergoing a HIV anti-retroviral therapy can stop taking their anti-retroviral therapy for at least one week.
28. The use of the composition of any of paragraphs 1 to 25, wherein the subject undergoing a HIV anti-retroviral therapy can stop taking their anti-retroviral therapy for at least one month.
29. A method of enhancing efficacy of at least one antiretroviral HIV therapy in a subject, the method comprising administering to the subject a pharmaceutical composition comprising at least one HIV polypeptide or fragment thereof and at least residues 34-288 of the N-terminal Bacillus anthracis Lethal Factor (LFn) polypeptide.
30. A method of enhancing efficacy of at least one antiretroviral HIV therapy in a subject, the method comprising administering to the subject a pharmaceutical composition comprising any of paragraphs 1-23 to a the subject.
31. The method of paragraphs 29 or 30, wherein the subject is a human subject.
32. The method of any of paragraphs 29 to 31, wherein the human subject is HIV positive or has AIDS.
33. The method of any of paragraphs 29 to 32, wherein the human subject has been exposed to HIV.
34. The method of any of paragraphs 29 to 33, wherein said composition is administered to a subject in combination before, after or concurrently with traditional antiretroviral therapies.
35. The method of any of paragraphs 29 to 34, wherein said composition is administered on a periodic basis to the subject.
36. The method of any of paragraphs 29 to 35, wherein said composition is administered to the subject at least yearly.
37. The method of any of paragraphs 29 to 36, wherein said composition is administered to the subject at least twice a year.
38. The method of any of paragraphs 29 to 37, wherein said composition is administered to the subject at least quarterly.
39. The method of any of paragraphs 29 to 38, wherein said composition is administered to the subject at least monthly.
40. The method of any of paragraphs 29 to 39, wherein said composition is administered to the subject more than once a month.
41. The method of any of paragraphs 29 to 40, wherein said HIV antigenic polypeptide is conjugated to the LFn polypeptide.
42. The method of any of paragraphs 29 to 41, wherein said HIV antigenic polypeptide is present in a fusion protein with the LFn polypeptide.
43. The method of any of paragraphs 29 to 42, wherein the subject can take breaks from traditional antiretroviral therapy.
44. The method of any of paragraphs 29 to 43, wherein the subject does not need to rigorously follow to the traditional antiretroviral therapy regimen.
45. The method any of paragraphs 29 to 44, wherein the subject does not need to rigorously follow to the traditional antiretroviral therapy regimen.
46. The method of any of paragraphs 29 to 45, wherein the subject can reduce the dose of their anti-retroviral therapy regimen.
47. The method of any of paragraphs 29 to 46, wherein the subject can miss an occasional dose of their anti-retroviral therapy regimen.
48. The method of any of any of paragraphs 29 to 47, wherein the subject can stop taking their anti-retroviral therapy for at least one week.
49. The method of any of paragraphs 29 to 48, wherein the subject can stop taking their anti-retroviral therapy for at least one month.

Unless otherwise explained, all technical and scientific terms used herein have the same meaning as commonly understood by one of ordinary skill in the art to which this disclosure belongs. Definitions of common terms in immunology and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); The ELISA guidebook (Methods in molecular biology 149) by Crowther J. R. (2000); Fundamentals of RIA and Other Ligand Assays by Jeffrey Travis, 1979, Scientific Newsletters; Immunology by Werner Luttmann, published by Elsevier, 2006. Definitions of common terms in molecular biology can also be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise stated, the present invention was performed using standard procedures, as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Methods in Enzymology: Guide to Molecular Cloning Techniques Vol. 152, S. L. Berger and A. R. Kimmerl Eds., Academic Press Inc., San Diego, USA (1987)); Current Protocols in Molecular Biology (CPMB) (Fred M. Ausubel, et al. ed., John Wiley and Sons, Inc.); Current Protocols in Protein Science (CPPS) (John E. Coligan, et. al., ed., John Wiley and Sons, Inc.); Current Protocols in Immunology (CPI) (John E. Coligan, et. al., ed. John Wiley and Sons, Inc.); Current Protocols in Cell Biology (CPCB) (Juan S. Bonifacino et. al. ed., John Wiley and Sons, Inc.); Culture of Animal Cells: A Manual of Basic Technique by R. Ian Freshney, Publisher: Wiley-Liss; 5th edition (2005), and Animal Cell Culture Methods (Methods in Cell Biology, Vol. 57, Jennie P. Mather and David Barnes editors, Academic Press, 1st edition, 1998) which are all incorporated by reference herein in their entirety.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

The examples presented herein relate to compositions comprising a LFn polypeptide or fragment thereof and a HIV antigen to augment conventional HIV anti-retroviral treatment in a subject with HIV. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Materials and Methods

Study Design. Eligible adult male and female HIV-1 infected volunteers were recruited from the HIV clinic at the Joint Clinical Research Centre (JCRC) in Kampala, Uganda. Recruitment was restricted to individuals with documentation of HIV status and evidence of HIV anti-retroviral therapy (ART)-mediated viral suppression for at least six months. Study eligibility criteria included: aged 18 to 60 years, CD4+ T cell counts >400, normal complete blood count, chemistry, liver function tests and urinalysis. All female volunteers had a negative pregnancy test at baseline, agreed not be breast-feeding and to use adequate birth control during the course of the study. All volunteers provided written informed consent. Reactogenicity and adverse events were assessed at 1 hour and 3 days after each immunization. Subsequent follow-up visits were 6, 9 and 12 months after enrollment in Phase 1A. After twelve months, volunteers from Phase 1A were invited to enroll in a follow-up Phase 1B trial where a 4-week observed treatment interruption was initiated following a single LFn-p24C booster immunization (Table 1). HIV plasma RNA, CD4 count and clinical evaluation were performed every fourteen days. After 4 weeks (28 days) of treatment interruption, participants were asked to resume their prior antiretroviral treatment regimens and were carefully monitored every two weeks for 2 months and at three and six months. For antiretroviral treatment that included medications with a short plasma half-life drug concentration, all antiretrovirals were stopped simultaneously and restarted within 4 weeks. For antiretroviral treatments that included a single long acting reagent (such as Nevirapine or Efavirenz), staggered interruption was initiated with the long acting agent being discontinued 7-10 days prior to discontinuation of other ART.

Counseling sessions were conducted at each study visit to assess ART adherence, and HIV risk behavior. Safety laboratory testing was done throughout both phases of the study and blood samples designated for immunogenicity testing were collected. PBMC were isolated and cryopreserved at visit 11A in accordance with the study protocol. Because placebo and baseline samples were not included in the data analysis, the inventors included as historical controls thirty-one unvaccinated individuals (sample time: month 0 and month 12). These volunteers were recruited from the JCRC and enrolled in an observational longitudinal study where counseling and blood draws were obtained every three months. The historical control cohort has comparable clinical profile to that of study volunteers and all had CD4+ T cell counts greater than 400 with undetectable viremia on a stable regimen of ART for at least 6 months.

The protocol was approved by the US and JCRC IRBs, the Uganda National Council for Sciences and Technologies and the Uganda National Drug Authority.

TABLE 1
Visit designation Study Visit Schedule Phase
1A Visit. ART = anti-retroviral therapy.

| | | |
|---|---|---|
| Day 0 | 1A | First Immunization |
| Day 3-7 | 2A | Clinical evaluation |
| Day 14 | 3A | Laboratory & Clinical evaluation |
| Day 28 | 4A | Second Immunization |
| Day 31-35 | 5A | Clinical evaluation |
| Day 42 | 6A | Laboratory & Clinical evaluation |
| Day 84 | 7A | Third Immunization |
| Day 87-90 | 8A | Clinical evaluation |
| Day 98 | 9A | Laboratory & Clinical evaluation |
| Day 168 | 10A | Laboratory & Clinical evaluation |
| Day 365 | 11A | T cell profile evaluation |

Phase 1B

| | | |
|---|---|---|
| Day 0 | 1B | Booster Immunization |
| Day 3-7 | 2B | Clinical evaluation |
| Day 14 | 3B | Discontinuation of any NNRTI |
| Day 21 | 4B | Discontinuation of ART |
| Day 35 | 5B | Laboratory & Clinical evaluation |
| Day 49 | 6B | ART Resumption |
| Day 63 | 7B | Laboratory & Clinical evaluation |
| Day 77 | 8B | Laboratory & Clinical evaluation |
| Day 91 | 9B | Laboratory & Clinical evaluation |
| Day 105 | 10B | Laboratory & Clinical evaluation |
| Day 182 | 11B | Laboratory & Clinical evaluation |

Vaccine Candidate

The immunogen is composed of a combination of an anthrax-derived polypeptide lethal factor (LFn, from which the toxin domain has been removed) fused to a subtype C HIV-1 gag p24 protein. LFn-fusion proteins have been studied extensively as intracellular delivery agents because of their unique capability to translocate antigen across the cell membrane without affecting cell viability, using the classical MHC Class I and II pathways²⁷⁻²⁹. LFn-p24C was produced by Water Reed Army Institute of Research (WRAIR) in compliance with the US standard of Good Manufacturing Practices (GMP) and provided by Vaccine Technologies, Inc (VTI). The product underwent GLP (Good Laboratory Practices) grade animal toxicity studies and completed a US FDA approved Phase safety studies in HIV-1 negative healthy volunteers in Maryland conducted by WRAIR (Deborah Birx and Shirley Lecher, personal communications) and provided by Vaccine Technologies, Inc (VTI). The protein expression vector for LFn and its fusion derivatives is the pET28b plasmid developed by Novagen (Madison, Wis.)³⁰. The main features of this vector system include an inducible T7 promoter, an internal His.Tag for protein purification, and multiple cloning sites. The recombinant LFn is expressed in coli as an intracellular soluble protein with 6 tandem repeat Histidine (His) at its N-terminal end. The molecular weight of LFn is about 31 kiloDalton (1(D). The LFn-p24C was diluted and administered intramuscularly in a volume of 1 ml in a total dose of 300 μg with Alhydrogel adjuvant. A total of 3 injections were administered intramuscularly in the deltoid region at 0, 1, and 3 months for Phase 1A, followed by single booster immunization at enrollment in Phase 1B.

CFSE Proliferation Assay

Cell proliferation was determined by carboxyfluorescein diacetate succinimidyl ester (CFSE) dilution using the CellTrace™ CFSE Cell Proliferation Kit (Invitrogen, Carlsbad, Calif.), per the manufacturer's instructions. Cells were stimulated with peptides for five days at 37° C. and 5% CO₂, then harvested and stained for surface markers with the following antibodies: CD3 APC, CD4 PE, CD8 PerCp-Cy5.5 (BD Biosciences, San Jose, Calif.). Samples were analyzed on a LSRII flow cytometer (BD Biosciences, San Jose, Calif.). Dead cells were excluded from analysis using a violet excited viability dye (LIVE/DEAD Fixable Dead Cell Stain; Invitrogen). All flow analysis was performed using FlowJo software (TreeStar, Ashland, Oreg.). Proliferation was measured by the extent of CFSE dilution. Staphylococcal Enterotoxin B (SEB Sigma-Aldrich, St. Louis, Mo.) stimulation was used as a positive control. All evaluable samples demonstrated significant proliferation following SEB stimulation. Results with less than 1% background response, and greater than 5% SEB response were considered valid. Only data with a minimum of 10,000 acquired events of CD3+CD4+or CD3+CD8+were analyzed. Only results greater than twice the background values and more than 0.1% after subtraction of background were considered positive.

Immune Profile

Activation staining was performed by incubating PBMC with the following antibodies: CD3 AmCyan, CD4 APC-Cy7, CD8 PerCPCy5.5, HLADR FITC, CD38 PE, and PD-1 APC (BD Biosciences San Jose, Calif.), and. Dead cells were excluded from analysis using a violet excited viability dye (LIVE/DEAD Fixable Dead Cell Stain; Invitrogen). Immune activation was defined as the percent of CD38+ HLA DR+ T cells and PD-1 levels were defined as the percent expression of PD-1 APC on CD3⁺ CD8⁺ (or CD4+) T cells. Gating was standardized and set using fluorescence minus one controls for HLADR, CD38 and PD-1. Data were analyzed using FLOWJO software (TreeStar, Ashland, Oreg.). A minimum of 30,000 CD4⁺ cells per sample were acquired and analyzed on an LSRII flow cytometer (BD Biosciences, San Jose, Calif.)³¹.

Antigens

Peptides corresponding to the consensus subtype C Gag (122 peptides) were synthesized as 15 amino acids (a.a.) overlapping by 11 a.a. (NIH/NIAID repository). A single pool of overlapping peptides, corresponding to the amino acid sequence of the HCMV pp 65 protein (JPT Peptide Technologies) was used to detect human CMV-specific responses. The final concentration of individual peptides was 1 μg/ml per peptide.

Statistical Analysis

Statistical analyses were performed using Prism Version 4.0 (GraphPad Software Inc. San Diego, Calif.). Paired t-test was used to compare data between time points and Mann-Whitney tests were used to compare the differences between control and study groups. P values <0.05 were considered statistically significant.

### Example 1

Demographics

Screening and enrollment into Phase 1A occurred from April 2008 to September 2008. Of the 153 volunteers screened at the JCRC, 30 HIV positive volunteers were identified and enrolled (25 women and 5 men). The mean age of the volunteers was 41 years (range 29-55). All participants were stably suppressed on ART for 6 months or greater, had undetectable viral load (<400 copies/mL) and a mean CD4+ T cell count of 520 (range 400-1100). The mean age of the HIV positive, unvaccinated control group (N=31) was 45 years (range 22-55). The mean CD4+ T count of this control cohort was 540 (range 400-1370) and all maintained undetectable viral load on a stable regimen of ART. No significant differences in age and CD4+ T cell counts were found between the two groups (p>0.05, data not shown).

A total of 29 out of 30 volunteers completed the Phase 1A study. One individual relocated outside of the country and was not able to complete their last visit at 12 months. Twenty-seven of the thirty volunteers from Phase 1A agreed to participate in Phase IB. Of these, twenty-four fully evaluable volunteers received a booster immunization and underwent closely monitored treatment interruption twenty-one days after receiving the LFn-p24C booster injection.

### Vaccine Safety

Local and systemic reactogenicity for both Phase 1A and 1B is shown in FIG. 1. The most commonly reported local symptoms were local pain and tenderness at the site of injection. The systemic symptoms related to LFn-p24C were malaise, myalgia and arthralgia. These local and systemic events were mostly mild and usually resolved prior to the subsequent visit (within 3-14 days). The majority of the self reported symptoms were mild 24/840 (2.9%) or moderate 1/840 (0.001%). There were no severe adverse events attributable to the immunogen and none of the volunteers discontinued the study due to adverse events. Other events not considered related to the LFn-p24C included urinary tract infection, influenza infection, low back pain, pharyngitis, and acute malaria.

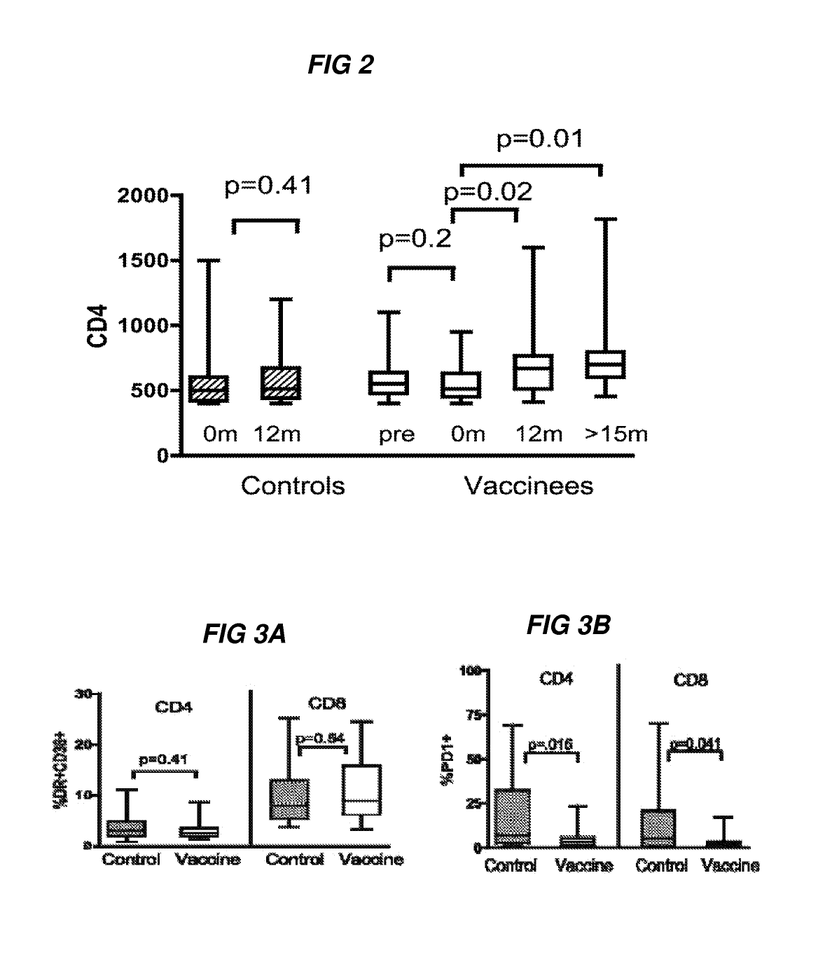

The inventors carefully monitored CD4 cell count and viral load closely after LFn-p24C administration throughout the course of the phase 1A study. All thirty volunteers continued to have undetectable viral loads at all evaluated time points throughout the duration of the phase 1A study. Administration of LFn-p24C was associated with a significant increase in CD4 cell count at and after 12 months compared to the unvaccinated control cohort (FIG. 2).

### Example 2

T Cell Profile of Vaccine Responders

HIV preferentially infects activated CD4+ T helper cells and this has previously raised concerns over whether an AIDS vaccine can generate more targets for the virus³²⁻³⁴, particularly in HIV infected individuals. The inventors next examined both CD8 and CD4 T-cell immune activation after three immunizations (visit 11A) and compared the levels to unvaccinated control samples. The inventors determined no significant differences in CD4 and CD8 immune activation between vaccine recipients and control samples (FIG. 3A, p>0.5).

Functional impairment of T cells during chronic HIV infection is associated with higher expression of programmed death 1 (PD-1), and upregulation of PD-1 is also predictor of disease progression³⁵⁻³⁷. Surprisingly, therapeutic immunization was associated with lower PD-1 expression in both CD4+and CD8+ T cells at visit 11A compared to unvaccinated control samples (FIG. 3B, p=0.016 and 0.041, respectively). No significant changes in the level of activation and PD-1 expression were observed in the unvaccinated control group within twelve months (p>0.5, data not shown).

### Example 3

### Vaccine-Specific T Cell Proliferation

HIV-1-specific T cell responses as measured by interferon secretion do not differ in individuals with progressive and long-term nonprogressive HIV-1 infection and are not directly associated with the level of viral replication³⁸⁻⁴⁰.

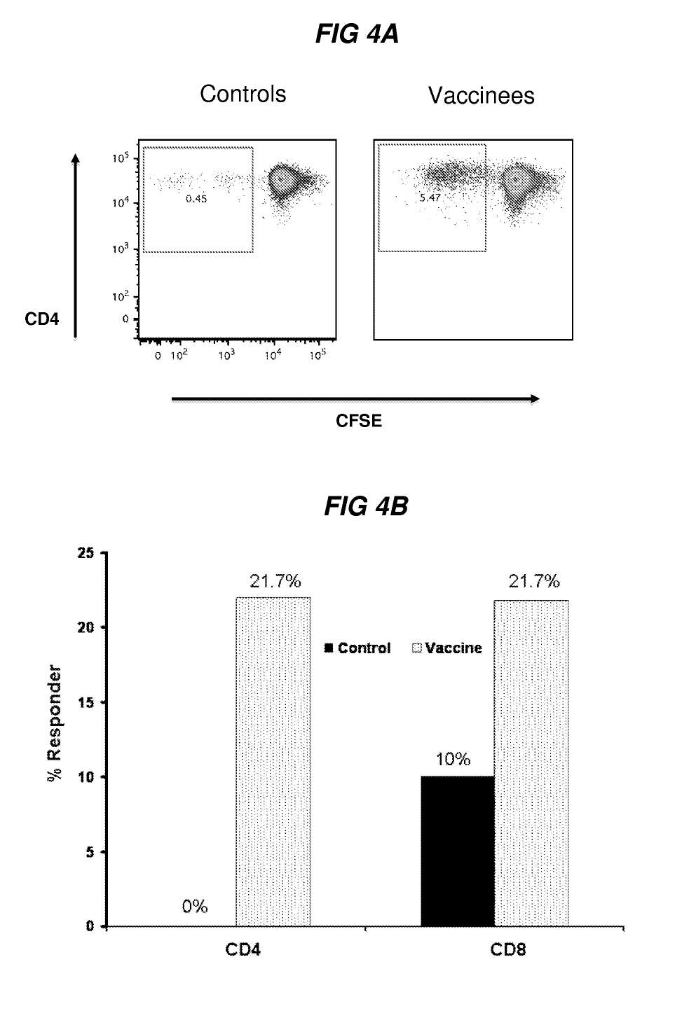

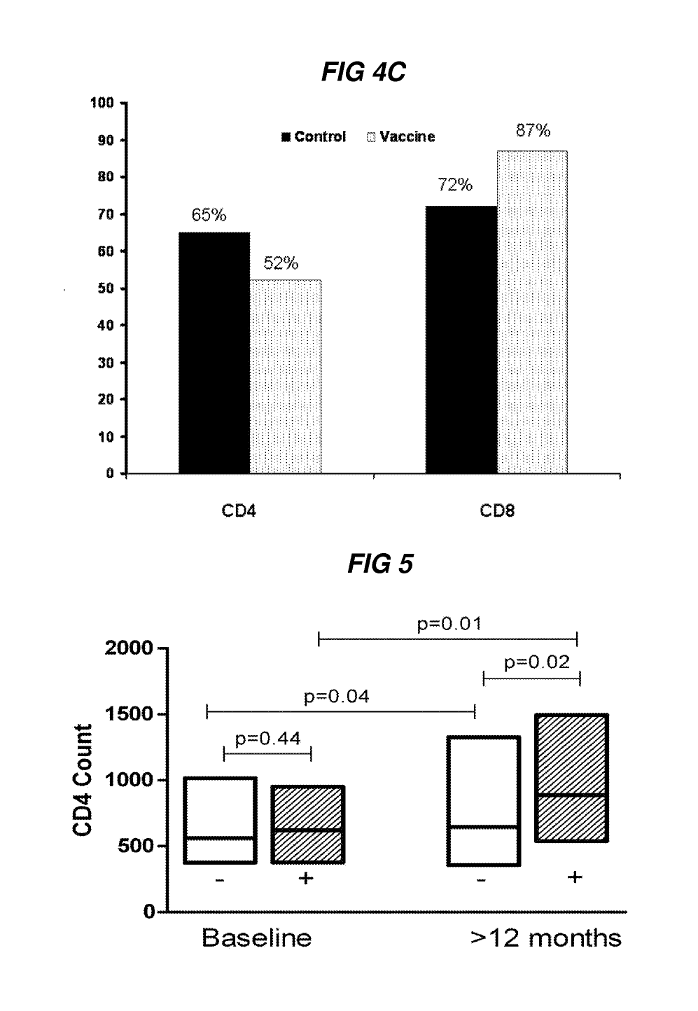

In contrast, HIV-1-specific proliferative responses are lost in individuals with progressive disease⁴¹. The innventors measured T cell proliferation in vaccine recipients after 3 immunizations (example of plot is shown in FIG. 4A). Flow-based proliferation, as measured by CFSE dilution, was measured in vaccine recipients at twelve months (visit 11A) and compared to unvaccinated controls. Results were valid in 23 vaccine and 20 control samples. Vaccine-specific CD4+ proliferation to Gag C was significantly higher in individuals who received the vaccine compared to the unvaccinated control group and 5/23 [21.7%] and 0/20[0%], respectively, (FIG. 4B, p<0.05). No CD4-mediated proliferation was detected in the control group at the two evaluated time points (12 months, data not shown).

In contrast, no significant differences in CMV-specific responses were observed between 12/23 [52.2%] of vaccine recipients and 13/20 [65%] control samples, respectively. Similarly, higher CD8+ vaccine-specific responses in 5/23 [21.7%] of vaccine recipients compared to 2/20 [10%] control samples, respectively (FIG. 4B). No significant differences in CMV-specific, CD8- and CD4-mediated responses were detected between the two groups (FIG. 4C, p>0.5%). Individuals with detectable vaccine-specific responses (CD4- and/or CD8-mediated) achieved a greater increase in CD4+ T cell count after three immunizations compared to vaccine recipients without detectable responses (FIG. 5).

### Example 4

### Structured Treatment Interruption

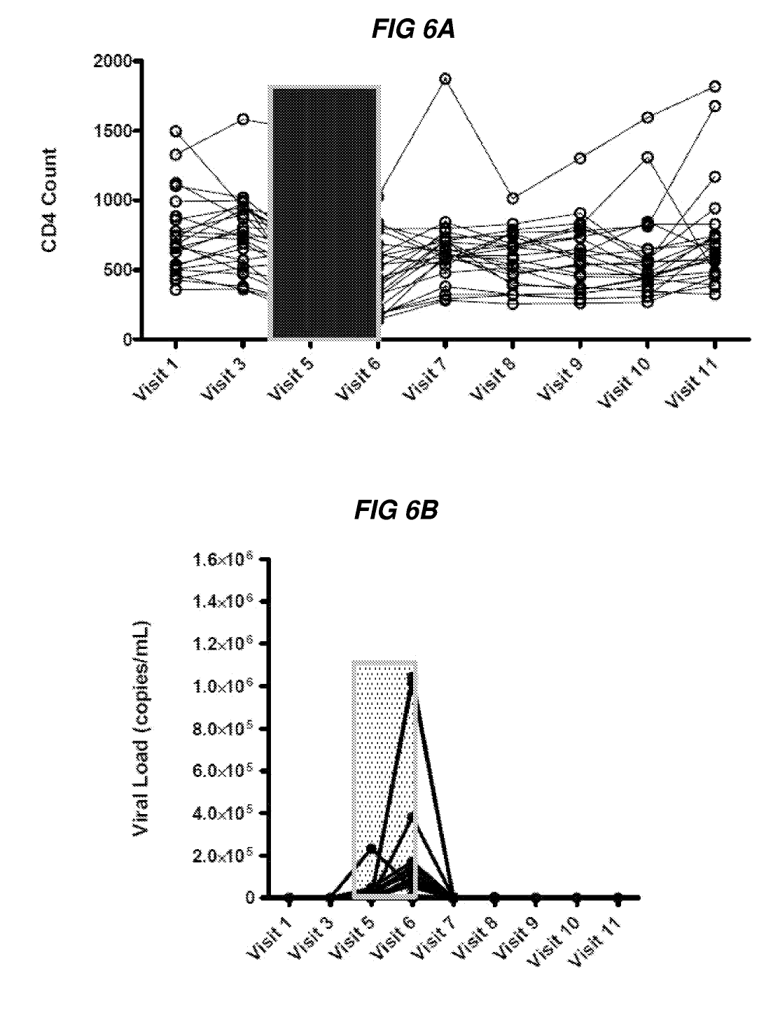

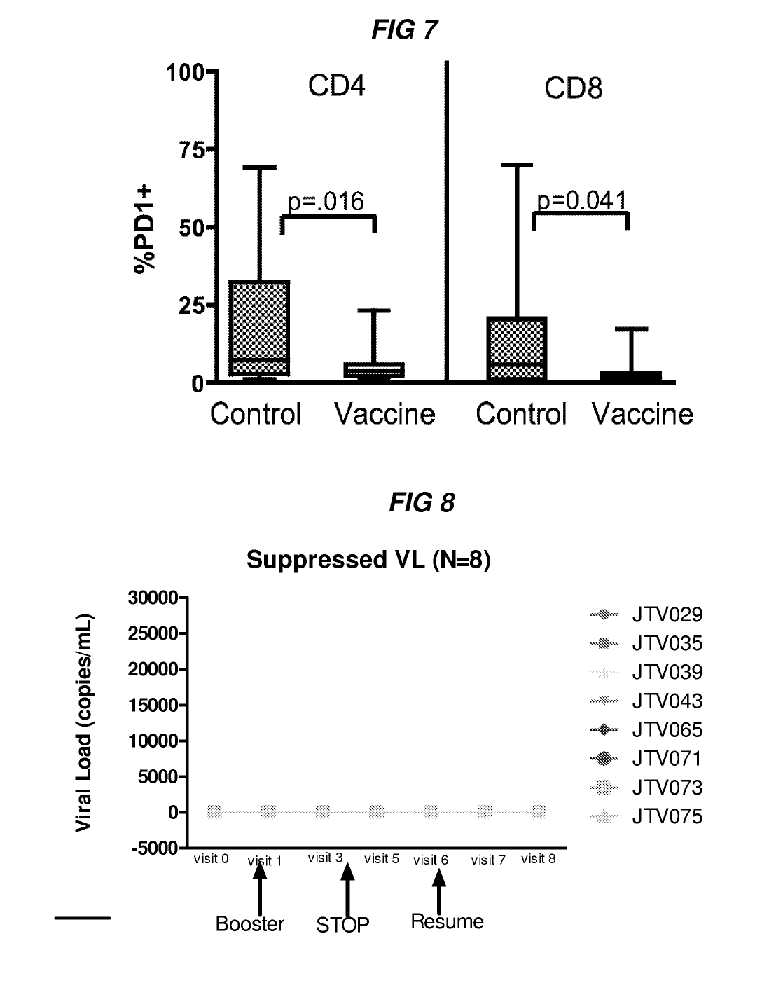

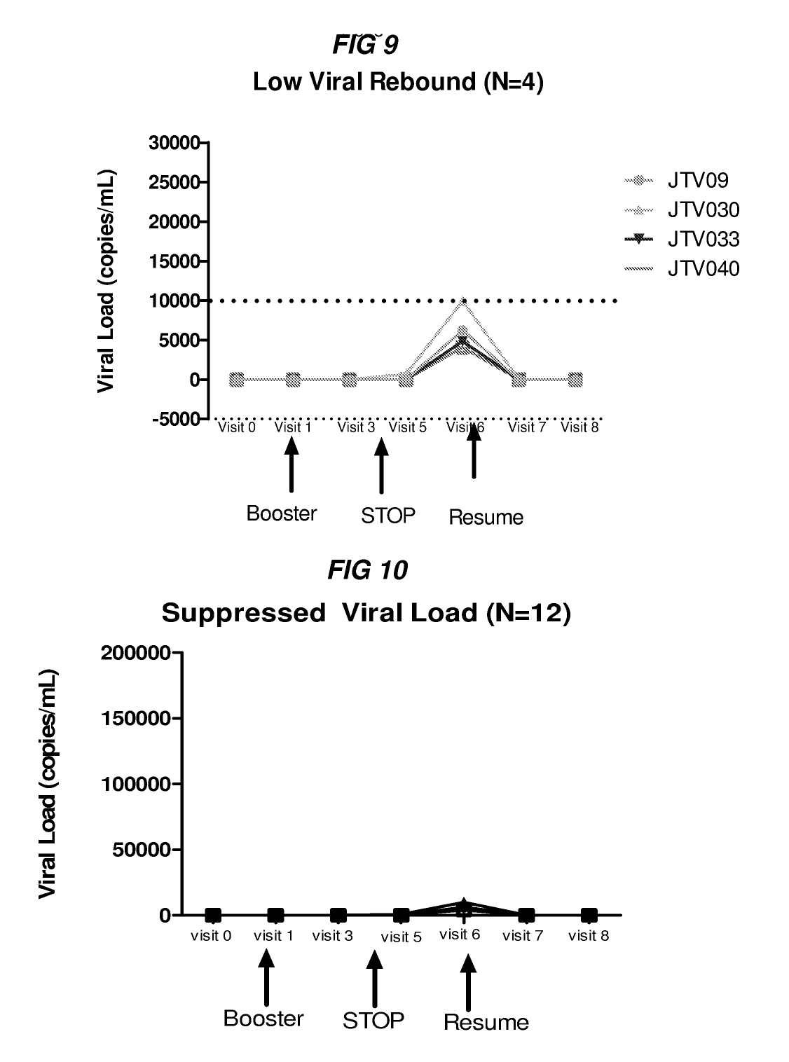

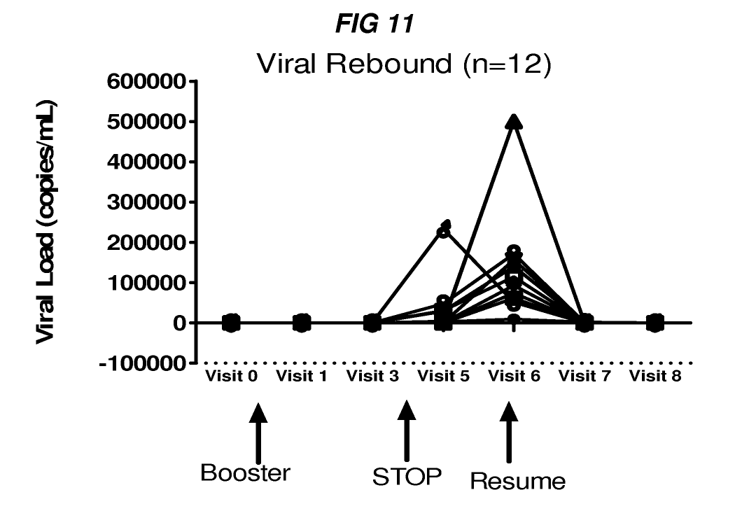

In order to assess whether therapeutic vaccination can elicit an anti-HIV response that controls viral replication, volunteers who completed Phase 1A were subsequently asked to undergo monitored treatment interruption following a single booster immunization. Twenty-one days after receiving a fourth dose of LFn-p24C, volunteers were instructed not to take their ART (ART was not dispensed), but to continue with any other medication they were currently being prescribed. Viral rebound was observed two weeks after treatment interruption was initiated. Full suppression of viremia followed ART resumption (FIG. 6A). CD4 cell counts were closely monitored throughout the course of this study (FIG. 6B). An anticipated decrease in CD4 cell count was observed following treatment interruption and full recovery of CD4 to baseline was not achieved by visit 11B (six months after booster immunization). Eight individuals (33%) showed no evidence of viral rebound during treatment interruption and these individuals had no significant decline in CD4 cell count (p=0.45, data not shown). Lack of viral rebound was not associated with T cell proliferation detected at visit 11A (data not shown).

The collapse of the immune system following HIV infection stems largely from the continual destruction of T cells. Antiretroviral treatment can restore CD4⁺ T cells but requires lifelong use and full adherence to medication regimens. Anti-retroviral therapy (ART) is also associated with potential side effects and secondary options remain costly for the majority of infected persons worldwide⁴². Established viral latency raises the possibility that complete eradication of HIV may be impossible with antiretroviral drugs alone. The underlying consideration in therapeutic vaccination is that boosting immunity is beneficial to the individual on ART and may modify the natural history of HIV disease favorably. Based on our hypothesis that an effective therapeutic approach needs to elicit potent antiviral immune responses, the inventors investigated the performance of LFn-p24C vaccine in healthy HIV positive Ugandans who maintained a stable regimen of antiretroviral treatment. Individuals who received LFn-p24C demonstrated a significant increase in CD4+ T cell counts over twelve months compared to the unvaccinated historical control group.

Vaccinations can augment HIV-1-specific T cell responses in chronically infected persons sufficiently to achieve significant effects on viral load during ART interruption^{43,44}. Our data demonstrate that therapeutic immunization induces HIV-specific T-helper and effector responses consistent with earlier studies^{26,45,46}. HIV-1-proliferative responses were associated with a greater increase in CD4+ T cell gain over 12 months. This would be consistent with other reports, which have shown that the preservation of the T cell proliferation capacity was generally associated with an apparently effective immune response in patients with HIV-1 infection. The inventors limited our study to asymptomatic, seropositive individuals on a stable ART regimens, who had CD4⁺ T cell counts >400 for optimal results from immunization⁴⁷⁻⁴⁹.

The functional impairment of T cells during chronic HIV infection is associated with T cell exhaustion. Dysfunctional T cells subsequently fail to eliminate the virus. However, the mechanism leading to this functional impairment has yet to be understood. PD-1 belongs to the B7:CD28 family, and plays an active and reversible role in virus-specific T cell exhaustion^{36,37,51,52} Blockade of the PD-1 pathway restores HIV-specific T cell function in HIV infection^{37,51}. In this cohort of chronically HIV-1 positive individuals, the combination of HIV-1 therapeutic vaccine and suppressive ART was associated with a decrease in PD-1 expression, a marker of immune dysfunction. Further evaluation of the role of vaccination in modulating immune dysfunction may provide important insight into the mechanism of viral induced immune impairment.

There are hints that therapeutic immunization may play a role as the era of highly active antiretroviral therapy progresses. Immune-based therapies in this population have the potential to be a crucial addition to currently available ART, particularly in regions where secondary ART options are limited. How does vaccine-specific T cell immunity prevent disease progression in chronic HIV infection? The presentation of antigens by a therapeutic immunogen may induce a distinct functional quality of the T cell that is associated with protective antiviral immunity, unlike the immune responses detected in HIV infection in the presence of viral suppression. In our study, volunteers who have evidence of vaccine-specific responses appear to gain significantly more CD4 T cells. However, these same individuals subsequently failed to control viremia during the scheduled treatment interruption, perhaps suggesting that discernable mechanisms may contribute to immune recovery and viral control.

Few controlled studies on the clinical effectiveness of therapeutic immunization in HIV infected individuals exists, particularly in Africa. Our trial examined the safety and efficacy of a therapeutic vaccine in HIV-1-infected Ugandans who are virally suppressed. The inventors demonstrate here that therapeutic immunization with LFn-p24C is safe. The inventors demonstrate that immunization improves CD4 count and boosts T cell responses in chronically HIV-1-infected volunteers.

### REFERENCES

The contents of all references cited throughout this application are incorporated herein by reference.
1. Kiguba, R., et al. Discontinuation and Modification of Highly Active Antiretroviral Therapy in HIV-Infected Ugandans: Prevalence and Associated Factors. JAcquir Immune Defic Syndr (2007).
2. Weidle, P. J., et al. Assessment of a pilot antiretroviral drug therapy programme in Uganda: patients' response, survival, and drug resistance. Lancet 360, 34-40 (2002).
3. Bartlett, J. A., et al. Minimizing resistance consequences after virologic failure on initial combination therapy: a systematic overview. J Acquir Immune Defic Syndr 41, 323-331 (2006).
4. Low, A. & Markowitz, M. Predictors of response to highly active antiretroviral therapy. AIDS Read 16, 425-428, 430-421, 434, 436 (2006).
5. Sabin, C. A., et al. Treatment exhaustion of highly active antiretroviral therapy (HAART) among individuals infected with HIV in the United Kingdom: multicentre cohort study. Bmj 330, 695 (2005).
6. Ledergerber, B., et al. Predictors of trend in CD4-positive T-cell count and mortality among HIV-1-infected individuals with virological failure to all three antiretroviral-drug classes. Lancet 364, 51-62 (2004).
7. Dube, M. P., et al. Glucose metabolism, lipid, and body fat changes in antiretroviral-naive subjects randomized to nelfinavir or efavirenz plus dual nucleosides. Aids 19, 1807-1818 (2005).
8. Lo, J. C., et al. The relationship between nucleoside analogue treatment duration, insulin resistance, and fasting arterialized lactate level in patients with HIV infection. Clin Infect Dis 41, 1335-1340 (2005).
9. Mulligan, K., et al. Altered fat distribution in HIV-positive men on nucleoside analog reverse transcriptase inhibitor therapy. J Acquir Immune Defic Syndr 26, 443-448 (2001).
10. Brinkman, K., Delnoy, P. P. & de Pauw, B. Highly active antiretroviral therapy (HAART) and prolonged survival of a patient with an HIV-related Burkitt lymphoma, despite an intracardiac relapse. Int J STD AIDS 9, 773-775 (1998).
11. Schambelan, M., et al. Management of metabolic complications associated with antiretroviral therapy for HIV-1 infection:
   recommendations of an International AIDS Society-USA panel. J Acquir Immune Defic Syndr 31, 257-275 (2002).
12. Deeks, S. G. The risk of treatment versus the risk of HIV replication. Lancet 367, 1955-1956 (2006).
13. Deeks, S. G. Antiretroviral treatment of HIV infected adults. Bmj 332, 1489 (2006).
14. Imami, N., Hardy, G. & Gotch, F. Development of immunotherapeutic strategies for HIV-1. Expert Opin Biol Ther 1, 803-816 (2001).
15. Kalams, S. A., et al. Levels of human immunodeficiency virus type 1-specific cytotoxic T-lymphocyte effector and memory responses decline after suppression of viremia with highly active antiretroviral therapy. J Virol 73, 6721-6728 (1999).
16. Pontesilli, O., et al. Functional T cell reconstitution and human immunodeficiency virus-1-specific cell-mediated immunity during highly active antiretroviral therapy. J Infect Dis 180, 76-86 (1999).
17. Jansen, C. A., et al. Long-term highly active antiretroviral therapy in chronic HIV-1 infection: evidence for reconstitution of antiviral immunity. Antivir Ther 11, 105-116 (2006).
18. Hardy, G. A., et al. Reconstitution of CD4+ T cell responses in HIV-1 infected individuals initiating highly active antiretroviral therapy (HAART) is associated with renewed interleukin-2 production and responsiveness. Clin Exp Immunol 134, 98-106 (2003).
19. Valdez, H., et al. HIV long-term non-progressors maintain brisk CD8 T cell responses to other viral antigens. Aids 16, 1113-1118 (2002).
20. Douek, D. C., et al. HIV preferentially infects HIV-specific CD4+ T cells. Nature 417, 95-98 (2002).
21. Rosenberg, E. S. & Walker, B. D. HIV type 1-specific helper T cells: a critical host defense. AIDS Res Hum Retroviruses 14 Suppl 2, 5143-147 (1998).
22. Lu, W., Wu, X., Lu, Y., Guo, W. & Andrieu, J. M. Therapeutic dendritic-cell vaccine for simian AIDS. Nat Med 9, 27-32 (2003).
23. Lu, W., Arraes, L. C., Ferreira, W. T. & Andrieu, J. M. Therapeutic dendritic-cell vaccine for chronic HIV-1 infection. Nat Med 10, 1359-1365 (2004).
24. Levy, Y., et al. Immunological and virological efficacy of a therapeutic immunization combined with interleukin-2 in chronically HIV-1 infected patients. Aids 19, 279-286 (2005).
25. MacGregor, R. R., et al. Plasmid vaccination of stable HIV-positive subjects on antiviral treatment results in enhanced CD8 T-cell immunity and increased control of viral “blips”. Vaccine 23, 2066-2073 (2005).
26. Moss, R. B., et al. Cell-mediated immune responses to autologous virus in HIV-1-seropositive individuals after treatment with an HIV-1 immunogen. Aids 14, 2475-2478 (2000).
27. Kushner, N., et al. A fragment of anthrax lethal factor delivers proteins to the cytosol without requiring protective antigen. Proc Natl Acad Sci USA 100, 6652-6657 (2003).
28. McEvers, K., et al. Modified anthrax fusion proteins deliver HIV antigens through MHC Class I and II pathways. Vaccine 23, 4128-4135 (2005).
29. Cao, H., et al. Delivery of exogenous proteins antigens to major histocompatibility complex class I pathway in cytosol. J. Infec Dis 185, 244-251 (2002).
30. Lu, Y., et al. Free in PMC Genetically modified anthrax lethal toxin safely delivers whole HIV protein antigens into the cytosol to induce T cell immunity. Proc. Natl. Acad. Sci. USA 97, 8027-8032 (2000).
31. Baker, C. A., et al. Profile of Immunologic Recovery in HIV-Infected Ugandan Adults after Antiretroviral Therapy. AIDS Res Hum Retroviruses 23, 900-905 (2007).
32. Castro, P., et al. Influence of a vaccination schedule on viral load rebound and immune responses in successfully treated HIV-infected patients. AIDS Res Hum Retroviruses 25, 1249-1259 (2009).
33. Jones, L. E. & Perelson, A. S. Modeling the effects of vaccination on chronically infected HIV-positive patients. J Acquir Immune Defic Syndr 31, 369-377 (2002).
34. Stanley, S. K., et al. Effect of immunization with a common recall antigen on viral expression in patients infected with human immunodeficiency virus type 1. N Engl J Med 334, 1222-1230 (1996).
35. D'Souza, M., et al. Programmed death 1 expression on HIV-specific CD4+ T cells is driven by viral replication and associated with T cell dysfunction. J Immunol 179, 1979-1987 (2007).
36. Day, C. L., et al. PD-1 expression on HIV-specific T cells is associated with T-cell exhaustion and disease progression. Nature 443, 350-354 (2006).
37. Kaufmann, D. E. & Walker, B. D. Programmed death-1 as a factor in immune exhaustion and activation in HIV infection. Curr Opin HIV AIDS 3, 362-367 (2008).
38. Addo, M. M., et al. Comprehensive epitope analysis of human immunodeficiency virus type 1 (HIV-1)-specific T-cell responses directed against the entire expressed HIV-1 genome demonstrate broadly directed responses, but no correlation to viral load. J Virol 77, 2081-2092 (2003).
39. Draenert, R., et al. Persistent recognition of autologous virus by high-avidity CD8 T cells in chronic, progressive human immunodeficiency virus type 1 infection. J Virol 78, 630-641 (2004).
40. Betts, M. R., et al. Analysis of total human immunodeficiency virus (HIV)-specific CD4(+) and CD8(+) T-cell responses: relationship to viral load in untreated HIV infection. J Virol 75, 11983-11991 (2001).
41. Migueles, S. A., et al. HIV-specific CD8+ T cell proliferation is coupled to perforin expression and is maintained in nonprogressors. Nat Immunol 3, 1061-1068 (2002).
42. Autran, B., et al. Positive effects of combined antiretroviral therapy on CD4+ T cell homeostasis and function in advanced HIV disease. Science 277, 112-116 (1997).
43. Levy, Y., et al. Sustained control of viremia following therapeutic immunization in chronically HIV-1-infected individuals. Aids 20, 405-413 (2006).
44. Levy, Y., et al. Immunological and virological efficacy of a therapeutic immunization combined with interleukin-2 in chronically HIV-1 infected patients. AIDS 19, 279-286 (2005).
45. Robbins, G. K., et al. Augmentation of HIV-1-specific T helper cell responses in chronic HIV-1 infection by therapeutic immunization. Aids 17, 1121-1126 (2003).
46. Maino, V. C., et al. Enhancement of HIV type 1 antigen-specific CD4+ T cell memory in subjects with chronic HIV type 1 infection receiving an HIV type 1 immunogen. AIDS Res Hum Retroviruses 16, 539-547 (2000).
47. Valentine, F. T., et al. A randomized, placebo-controlled study of the immunogenicity of human immunodeficiency virus (HIV) rgp160 vaccine in HIV-infected subjects with > or =400/mm3 CD4 T lymphocytes (AIDS Clinical Trials Group Protocol 137). J Infect Dis 173, 1336-1346 (1996).
48. Davey, R. T., Jr., et al. Immunologic and virologic effects of subcutaneous interleukin 2 in combination with antiretroviral therapy: A randomized controlled trial. JAMA 284, 183-189 (2000).
49. Lange, C. G., et al. Nadir CD4+ T-cell count and numbers of CD28+CD4+ T-cells predict functional responses to immunizations in chronic HIV-1 infection. Aids 17, 2015-2023 (2003).
50. Hardy, G. A., et al. A phase I, randomized study of combined IL-2 and therapeutic immunisation with antiretroviral therapy. J Immune Based Ther Vaccines 5, 6 (2007).
51. Kaufmann, D. E. & Walker, B. D. PD-1 and CTLA-4 inhibitory cosignaling pathways in HIV infection and the potential for therapeutic intervention. J Immunol 182, 5891-5897 (2009).
52. Zhang, J. Y., et al. PD-1 up-regulation is correlated with HIV-specific memory CD8+ T-cell exhaustion in typical progressors but not in long-term nonprogressors. Blood 109, 4671-4678 (2007).

1.-23. (canceled)

24. A method of enhancing efficacy of at least one anti-retroviral HIV therapy in a subject, the method comprising administering to the subject a pharmaceutical composition comprising at least one HIV polypeptide or fragment thereof and at least residues 34-288 of the N-terminal Bacillus anthracis Lethal Factor (LFn) polypeptide.

25. (canceled)

26. The method of claim 24, wherein the subject is a human subject.

27. The method of claim 24, wherein the human subject is HIV positive or has AIDS, or has been exposed to HIV.

28. (canceled)

29. The method of claim 24, wherein said composition is administered to a subject in combination before, after or concurrently with at least one anti-retroviral therapy.

30. The method of claim 24, wherein said composition is administered on a periodic basis to the subject.

31. The method of claim 24, wherein said HIV antigenic polypeptide is conjugated to the LFn polypeptide or present in a fusion protein with the LFn polypeptide.

32. (canceled)

33. The method of claim 24, wherein the subject can take at least one break from the anti-retroviral therapy or can miss at least one dose of their anti-retroviral therapy regimen.

34. (canceled)

35. The method of claim 24, wherein the subject can reduce the dose of their anti-retroviral therapy regimen.

36. (canceled)

37. The method of claim 33, wherein the subject can stop taking their anti-retroviral therapy for at least one week.

38. The method of claim 33, wherein the subject can stop taking their anti-retroviral therapy for at least one month.

39. The method of claim 24, wherein an adjuvant selected from the group consisting of QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59 is added with the pharmaceutical composition.

40. The method of claim 24, wherein said LFn polypeptide comprises a conservative substitution variant thereof, that promotes transmembrane delivery to the cytosol of an intact cell.

41. The method of claim 24, wherein said LFn polypeptide is N-glycosylated.

42. The method of claim 24, wherein said LFn polypeptide comprises at least the 60 carboxy-terminal amino acids of SEQ. ID. No. 3, or a conservative substitution variant thereof.

43. The method of claim 24, wherein said LFn polypeptide comprises at least the 104 carboxy-terminal amino acids of SEQ. ID. No. 3, or a conservative substitution variant thereof.

44. The method of claim 24, wherein said LFn polypeptide comprises the amino acid sequence corresponding to SEQ. ID. No. 5, or a conservative substitution variant thereof.

45. The method of claim 24, wherein said LFn polypeptide substantially lacks the amino acids 1-33 of SEQ. ID. No. 3.

46. The method of claim 24, wherein said fragment of a HIV polypeptide is at least 15 amino acids.

47. The method of claim 24, wherein the at least one antiretroviral therapy is selected from any or a combination from the group consisting of: stem cell therapy, Tenofovir, Lamivudine, Zidovudine, Abacavir, zidovudine AZT, (S)-6-chloro-4-(cyclopropylethynyl)-1,4-dihydro-4-(trifluoromethyl)-2H-3,11-Cyclopropyl-5,11-dihydro-4-methyl-6H-dipyrido[3,2-b: 2′,3′-e][1,4]diazepin-6-one, or derivatives thereof.
